(19) United States
(12) Patent Application Publication
(10) Pub. No.: US 2024/0368178 A1
(43) Pub. Date: Nov. 7, 2024

(54) IMIDAZOCYCLIC COMPOUND AND APPLICATION THEREOF

(71) Applicant: DONGBAO PURPLE STAR (HANGZHOU) BIOPHARMACEUTICAL CO., LTD., Zhejiang (CN)

(72) Inventors: Qinghua MAO, Shanghai (CN); Wei WANG, Shanghai (CN); Ye TAN, Shanghai (CN); Bin LIANG, Shanghai (CN); Lu WANG, Shanghai (CN); Chengde WU, Shanghai (CN); Yang ZHANG, Shanghai (CN); Shuhui CHEN, Shanghai (CN)

(21) Appl. No.: 18/684,855

(22) PCT Filed: Aug. 24, 2022
(86) PCT No.: PCT/CN2022/114560

§ 371 Date: Feb. 20, 2024

(30) Foreign Application Priority Data
Aug. 24, 2021 (CN) 202110976089.5
Sep. 29, 2021 (CN) 202111154964.8
Sep. 30, 2021 (CN) 202111162621.6

(51) Int. Cl. C07D 495/04; A61K 31/444; A61K 31/454; A61K 31/4545; A61K 31/496

(52) U.S. Cl. CPC C07D 495/04; A61K 31/444; A61K 31/454; A61K 31/4545; A61K 31/496

(57) ABSTRACT

A series of imidazocyclic compounds and an application thereof, which specifically relate to a compound represented by formula (P) and a pharmaceutically acceptable salt thereof.

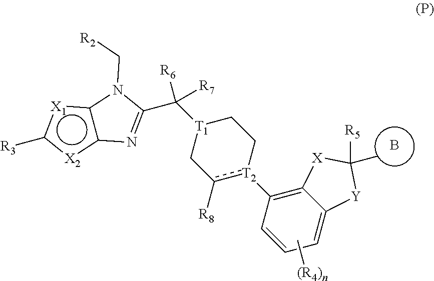

The present invention claims the right of the following priorities:
CN202110976089.5, application date: Aug. 24, 2021;
CN202111154964.8, application date: Sep. 29, 2021;
CN202111162621.6, application date: Sep. 30, 2021.

### TECHNICAL FIELD

The present disclosure relates to a series of imidazocyclic compounds and a use thereof, specifically to a compound of formula (P) and a pharmaceutically acceptable salt thereof.

### BACKGROUND

Type 2 diabetes mellitus (T2DM) is one of the most common chronic metabolic diseases, frequently occurring in middle-aged and elderly people and obese individuals, and seriously affecting patients' physical and mental health and quality of life. The main pathogenic mechanisms of type 2 diabetes mellitus are: pancreatic β-cell dysfunction, insulin resistance, abnormal glucagon secretion, etc. Common hypoglycemic drugs (such as insulin, sulfonylurea drugs, and thiazolidinedione drugs) mainly work through improving patients' pancreatic 3-cell secretion function or insulin resistance, but often cause adverse reactions such as hypoglycemia, which in turn have a negative impact on cardiovascular function. In recent years, diabetes-related clinical research guidelines have indicated that, in addition to glycemic control, clinical treatment of T2DM should also provide benefits for patients in terms of weight loss and cardiovascular health. Therefore, it is urgent to develop drugs with novel mechanisms of action for the treatment of T2DM.

Glucagon-like peptide-1 (GLP-1) is a hormone secreted by intestinal L cells after a meal, and is a polypeptide composed of 30 amino acids. After binding to the glucagon-like peptide-1 receptor (GLP-1R), GLP-1 enhances glucose-stimulated insulin secretion (GSIS) by upregulating downstream cAMP, MAPK, and other signals, promotes insulin secretion from pancreatic β cells, reduces the risk of hypoglycemia, promotes β cell proliferation, inhibits R cell apoptosis, inhibits glucagon secretion from pancreatic α cells, protects cardiovascular function, delays gastric emptying, and reduces food intake, thereby leading to weight loss. Therefore, the development of T2DM drugs based on GLP-1 is a new strategy, including GLP-1 analogs, GLP-1R agonists, and DPP-4 inhibitors.

GLP-1R agonists currently available in China, including liraglutide and exenatide, are essentially GLP-1 analogs, which can promote insulin secretion from pancreatic β-cells, protect the function of pancreatic β-cells, and have the obvious effect of reducing the body weight of the patients. However, these drugs need be administered subcutaneously or intravenously, which increases the risk of infection, has poor patient compliance and high costs, making them difficult to be widely applied in clinical practice. The only oral GLP-1R agonist, semaglutide, is of stringent administration conditions, high storage requirements, and high prices. Therefore, the development of more efficient, low-toxic small-molecule oral GLP-1R agonists has a better application prospect.

### CONTENT OF THE PRESENT INVENTION

The present disclosure provides a compound of formula (P) or a pharmaceutically acceptable salt thereof,

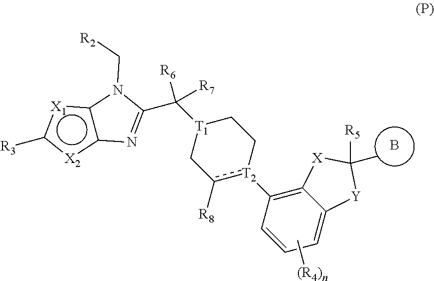

wherein
[symbol] is selected from a single bond and a double bond, and when T₂ is selected from N, [symbol] is selected from a single bond;
T₁ and T₂ are selected from N and CR₉;
X and Y are each independently selected from CH₂, O—CH₂, NH, O, and C(═O);
X₁ and X₂ are each independently selected from CH, N, O, and S, and the CH is optionally substituted by one F, Cl, Br, and CH₃;
ring B is selected from 5- to 6-membered heteroaryl, and the 5- to 6-membered heteroaryl is optionally substituted by 1, 2, or 3 R₁;
or, ring B is selected from phenyl, and the phenyl is optionally substituted by 1, 2, or 3 R₁, and in this case, X and Y are not simultaneously selected from O;
R₁ is selected from F, Cl, Br, I, OH, NH₂, CN, CH₃, and OCH₃;
R₂ is selected from

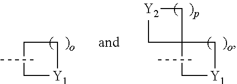

and the

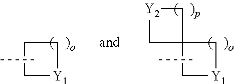

are optionally substituted by 1, 2, or 3 Rₐ;
Y₁ and Y₂ are each independently selected from CH₂, NH, and O;
and p are each independently selected from 0, 1, 2, and 3;
R₃ is selected from —C(═O)—NH—R_{b}, —C(═O)—R_{b}, —C(═O)—NH—S(═O)₂—R_{b}, —S(═O)₂—NH—R_{b}, —S(═O)₂—R_{b}, —P(═O)(R_{b})₂, C₁₋₃ alkyl, tetrazolyl, isoxazolyl,

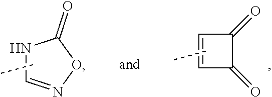

and the C₁₋₃ alkyl, tetrazolyl, isoxazolyl,

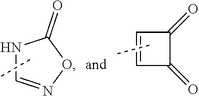

are optionally substituted by 1, 2, or 3 R_{b};
R₄ is selected from D, F, Cl, Br, I, and C₁₋₃ alkyl, and the C₁₋₃ alkyl is optionally substituted by 1, 2, or 3 R;
R₅ is selected from H, D, and CH₃, and the CH₃ is optionally substituted by 1, 2, or 3 R;
R₆, R₇, R₈, and R₉ are each independently selected from H, D, and C₁₋₃ alkyl, and the C₁₋₃ alkyl is optionally substituted by 1, 2, or 3 R;
n is selected from 0, 1, and 2;
each Rₐ is independently selected from F, Cl, Br, and I;
each R_{b} is independently selected from OH, CN, C₁₋₃ alkyl, C₁₋₃ alkoxy, C₁₋₃ alkylamino, and oxazolyl, and the C₁₋₃ alkyl, C₁₋₃ alkoxy, and oxazolyl are optionally substituted by 1, 2, or 3 R;
each R is independently selected from D, F, Cl, Br, and I.

In some embodiments of the present disclosure, the R_{b} is independently selected from OH, CN, CH₃, CF₃, and OCH₃, and other variables are as defined in the present disclosure.

In some embodiments of the present disclosure, the R₂ is selected from

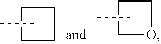

and the

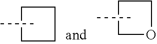

are optionally substituted by 1, 2, or 3 Rₐ, and other variables are as defined in the present disclosure.

In some embodiments of the present disclosure, the R₂ is selected from

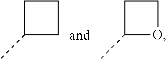

and other variables are as defined in the present disclosure.

In some embodiments of the present disclosure, the R₂ is selected from

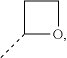

and other variables are as defined in the present disclosure.

In some embodiments of the present disclosure, the R₃ is selected from —COOH, —C(═O)—NH—CN, —C(═O)—NH—OH, —C(═O)—NH—OCH₃, —C(═O)—CF₃, —S(═O)₂—NH—CH₃, and —S(═O)₂—OH, and other variables are as defined in the present disclosure.

In some embodiments of the present disclosure, the R₃ is selected from —COOH, and other variables are as defined in the present disclosure.

In some embodiments of the present disclosure, the R₄ is selected from F and CH₃, and other variables are as defined in the present disclosure.

In some embodiments of the present disclosure, the R₅ is selected from H, D, CH₃, CF₃, CHF₂, CHD₂, and CD₃, and other variables are as defined in the present disclosure.

In some embodiments of the present disclosure, the R₅ is selected from CH₃, and other variables are as defined in the present disclosure.

In some embodiments of the present disclosure, the R₆, R₇, R₈, and R₉ are each independently selected from H, D, and CH₃, and the CH₃ is optionally substituted by 1, 2, or 3 R, and other variables are as defined in the present disclosure.

In some embodiments of the present disclosure, the R₆ is selected from H, D, and CH₃, and other variables are as defined in the present disclosure.

In some embodiments of the present disclosure, the R₇ is selected from H, D, and CH₃, and other variables are as defined in the present disclosure.

In some embodiments of the present disclosure, the R₈ is selected from H, D, and CH₃, and other variables are as defined in the present disclosure.

In some embodiments of the present disclosure, the R₉ is selected from H, D, and CH₃, and other variables are as defined in the present disclosure.

In some embodiments of the present disclosure, the ring B is selected from 6-membered heteroaryl, and the 6-membered heteroaryl is optionally substituted by 1, 2, or 3 R₁, and other variables are as defined in the present disclosure.

In some embodiments of the present disclosure, the ring B is selected from pyridyl, pyrimidinyl, pyrazinyl, pyridazinyl, pyrazolyl, imidazolyl, thiazolyl, and oxazolyl, and the pyridyl, pyrimidinyl, pyrazinyl, pyridazinyl, pyrazolyl, imidazolyl, thiazolyl, and oxazolyl are optionally substituted by 1, 2, or 3 R₁, and other variables are as defined in the present disclosure.

In some embodiments of the present disclosure, the ring B is selected from pyridyl, and the pyridyl is optionally substituted by 1, 2, or 3 R₁, and other variables are as defined in the present disclosure.

In some embodiments of the present disclosure, the ring B is selected from

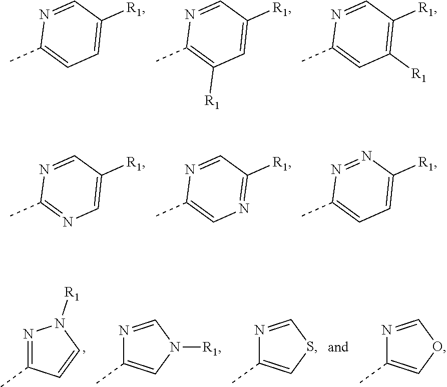

and other variables are as defined in the present disclosure.

In some embodiments of the present disclosure, the ring B is selected from

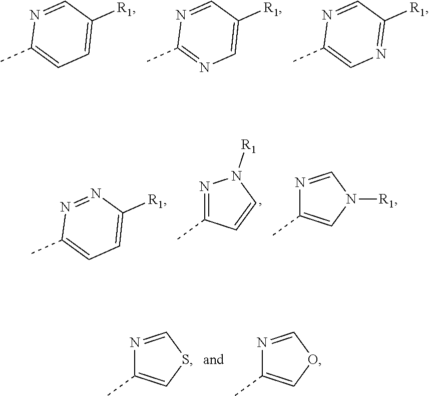

and other variables are as defined in the present disclosure.

In some embodiments of the present disclosure, the ring B is selected from

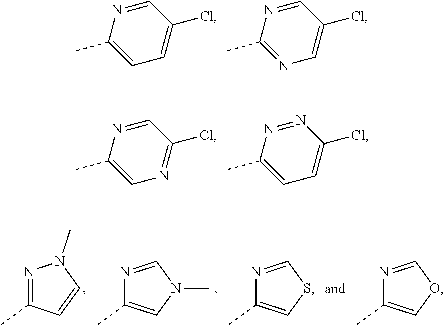

and other variables are as defined in the present disclosure.

In some embodiments of the present disclosure, the ring B is selected from

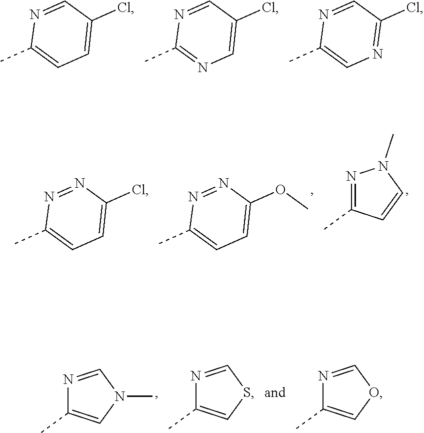

and other variables are as defined in the present disclosure.

In some embodiments of the present disclosure, the ring B is selected from

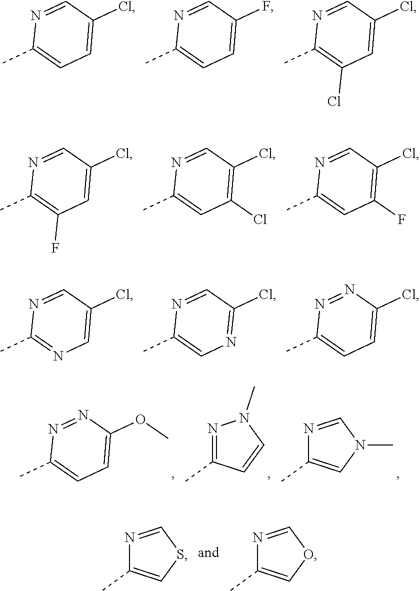

and other variables are as defined in the present disclosure.

In some embodiments of the present disclosure, the ring B is selected from

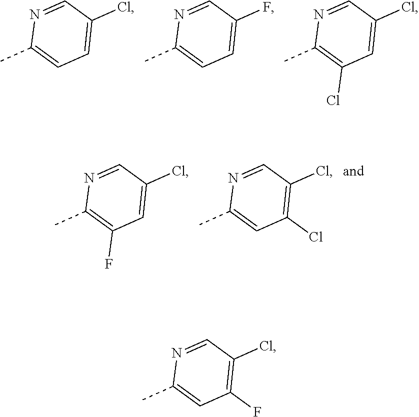

and other variables are as defined in the present disclosure.

In some embodiments of the present disclosure, the ring B is selected from

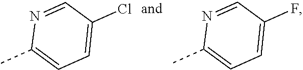

and other variables are as defined in the present disclosure.

In some embodiments of the present disclosure, the structural moiety

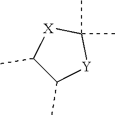

is selected from

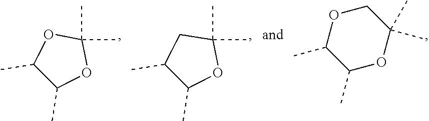

and other variables are as defined in the present disclosure.

In some embodiments of the present disclosure, the structural moiety

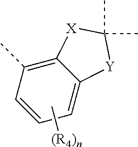

is selected from

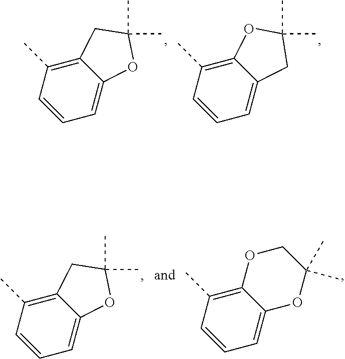

and other variables are as defined in the present disclosure.

In some embodiments of the present disclosure, the ring B is selected from phenyl, and the phenyl is optionally substituted by 1, 2, or 3 R₁, the structural moiety

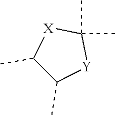

is selected from

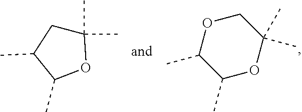

and other variables are as defined in the present disclosure.

In some embodiments of the present disclosure, the ring B is selected from

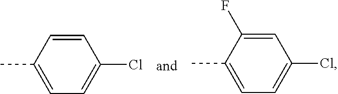

the structural moiety

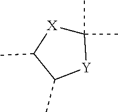

is selected from

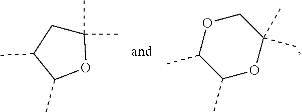

and other variables are as defined in the present disclosure.

In some embodiments of the present disclosure, the structural moiety

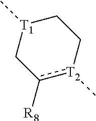

is selected from

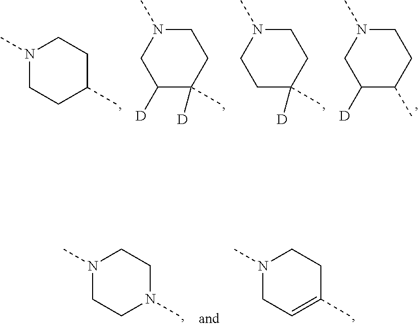

and other variables are as defined in the present disclosure.

In some embodiments of the present disclosure, the structural moiety

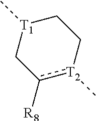

is selected from

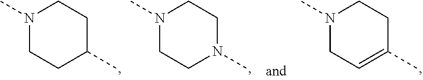

and other variables are as defined in the present disclosure.

In some embodiments of the present disclosure, the structural moiety

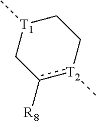

is selected from

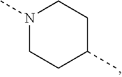

and other variables are as defined in the present disclosure.

In some embodiments of the present disclosure, the structural moiety

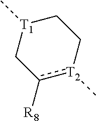

is selected from

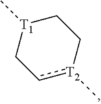

and other variables are as defined in the present disclosure.

In some embodiments of the present disclosure, the structural moiety

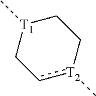

is selected from

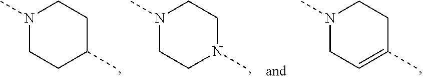

and other variables are as defined in the present disclosure.

In some embodiments of the present disclosure, the structural moiety

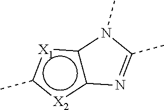

is selected from

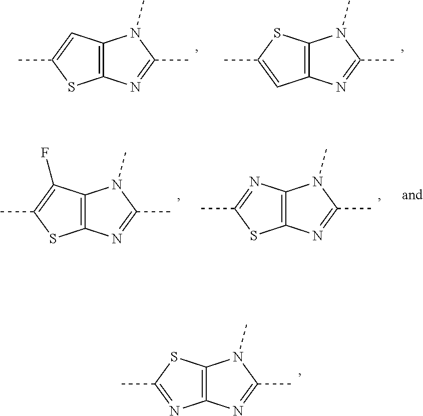

and other variables are as defined in the present disclosure.

In some embodiments of the present disclosure, the structural moiety

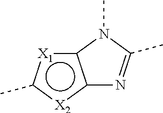

is selected from

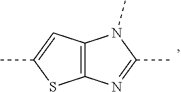

and other variables are as defined in the present disclosure.

In some embodiments of the present disclosure, the compound or the pharmaceutically acceptable salt thereof is selected from:

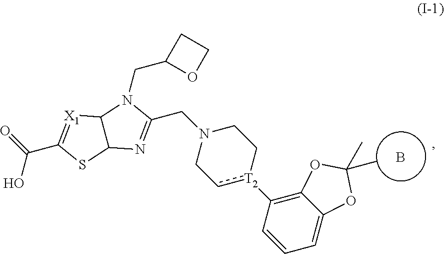

wherein
[symbol] is selected from a single bond and a double bond, and when T₂ is selected from N, [symbol] is selected from a single bond;
X₁, T₂, and ring B are as defined in the present disclosure.

In some embodiments of the present disclosure, the compound or the pharmaceutically acceptable salt thereof is selected from:

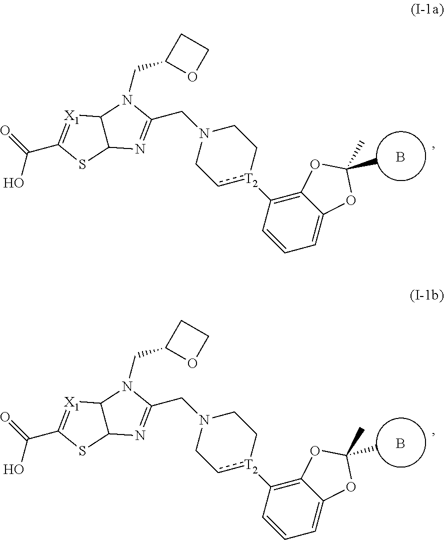

wherein
[symbol] is selected from a single bond and a double bond, and when T₂ is selected from N, [symbol] is selected from a single bond;
X₁, T₂, and ring B are as defined in the present disclosure.

The present disclosure further provides a compound of formula (I) or a pharmaceutically acceptable salt thereof,

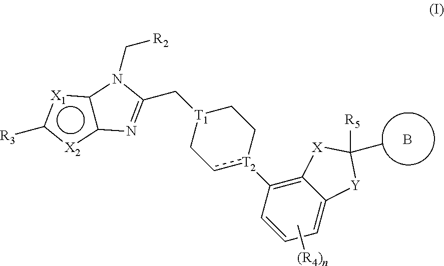

wherein
[symbol] is selected from a single bond and a double bond, and when T₂ is selected from N, [symbol] is selected from a single bond;
T₁ and T₂ are selected from N and CH;
X and Y are each independently selected from CH₂, O—CH₂, NH, O, and C(═O);
X₁ and X₂ are each independently selected from CH, N, O, and S, and the CH is optionally substituted by one F, Cl, Br, and CH₃;
ring B is selected from 5- to 6-membered heteroaryl, and the 5- to 6-membered heteroaryl is optionally substituted by 1, 2, or 3 R₁;
or, ring B is selected from phenyl, and the phenyl is optionally substituted by 1, 2, or 3 R₁, and in this case, X and Y are not simultaneously selected from O;
R₁ is selected from F, Cl, Br, I, OH, NH₂, CN, CH₃, and OCH₃;
R₂ is selected from

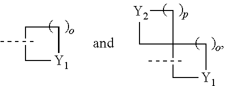

and the

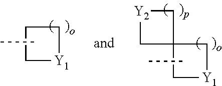

are optionally substituted by 1, 2, or 3 Rₐ;
Y₁ and Y₂ are each independently selected from CH₂, NH, and O;
o and p are each independently selected from 0, 1, 2, and 3;
R₃ is selected from —C(═O)—NH—R_{b}, —C(═O)—R_{b}, —C(═O)—NH—S(═O)₂—R_{b}, —S(═O)₂—NH—R_{b}, —S(═O)₂—R_{b}, —P(═O)(R_{b})₂, C₁₋₃ alkyl, tetrazolyl, isoxazolyl,

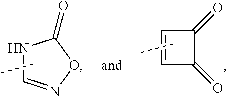

and the C₁₋₃ alkyl, tetrazolyl, isoxazolyl,

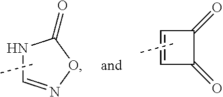

and are optionally substituted by 1, 2, or 3 R_{b};
R₄ is selected from F, Cl, Br, I, and C₁₋₃ alkyl;
R₅ is selected from H and CH₃;
n is selected from 0, 1, and 2;
each Rₐ is independently selected from F, Cl, Br, and I;
each R_{b} is independently selected from OH, CN, C₁₋₃ alkyl, C₁₋₃ alkoxy, C₁₋₃ alkylamino, and oxazolyl, and the C₁₋₃ alkyl, C₁₋₃ alkoxy, and oxazolyl are optionally substituted by 1, 2, or 3 R;
each R is independently selected from F, Cl, Br, and I.

The present disclosure further provides a compound of formula (I) or a pharmaceutically acceptable salt thereof,

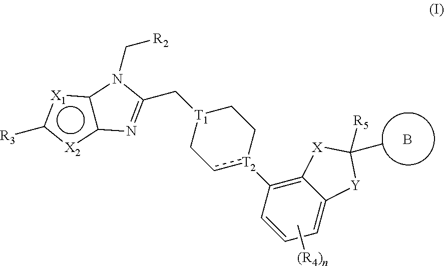

wherein
[symbol] is selected from a single bond and a double bond, and when T₂ is selected from N, [symbol] is selected from a single bond;
T₁ and T₂ are selected from N and CH;
X and Y are each independently selected from CH₂, NH, O, and C(═O);
X₁ and X₂ are each independently selected from CH, N, O, and S, and the CH is optionally substituted by one F, Cl, Br, and CH₃;
ring B is selected from 5- to 6-membered heteroaryl, and the 5- to 6-membered heteroaryl is optionally substituted by 1, 2, or 3 R₁;
R₁ is selected from F, Cl, Br, I, OH, NH₂, CN, CH₃, and OCH₃;
R₂ is selected from

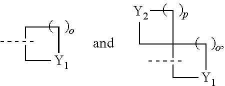

and the

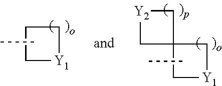

are optionally substituted by 1, 2, or 3 Rₐ;
Y₁ and Y₂ are each independently selected from CH₂, NH, and O;
o and p are each independently selected from 0, 1, 2, and 3;
R₃ is selected from —C(═O)—NH—R_{b}, —C(═O)—R_{b}, —C(═O)—NH—S(═O)₂—R_{b}, —S(═O)₂—NH—R_{b}, —S(═O)₂—R_{b}, —P(═O)(R_{b})₂, C₁₋₃ alkyl, tetrazolyl, isoxazolyl,

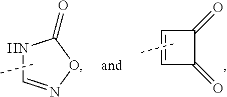

and the C₁₋₃ alkyl, tetrazolyl, isoxazolyl,

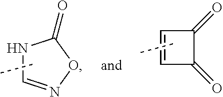

and are optionally substituted by 1, 2, or 3 R_{b};
R₄ is selected from F, Cl, Br, I, and C₁₋₃ alkyl;
R₅ is selected from H and CH₃;
n is selected from 0, 1, and 2;
each Rₐ is independently selected from F, Cl, Br, and I;
each R_{b} is independently selected from OH, CN, C₁₋₃ alkyl, C₁₋₃ alkoxy, C₁₋₃ alkylamino, and oxazolyl, and the C₁₋₃ alkyl, C₁₋₃ alkoxy, and oxazolyl are optionally substituted by 1, 2, or 3 R;
each R is independently selected from F, Cl, and Br.

The present disclosure further provides a compound of formula (II) or a pharmaceutically acceptable salt thereof,

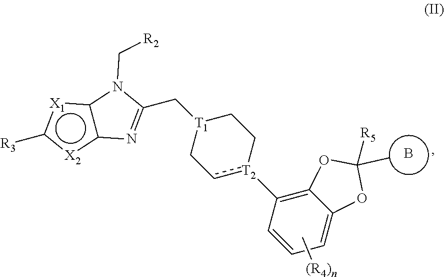

wherein
[symbol] is selected from a single bond and a double bond, and when T₂ is selected from N, [symbol] is selected from a single bond;
T₁ and T₂ are selected from N and CH;
X₁ and X₂ are each independently selected from CH, N, O, and S, and the CH is optionally substituted by one F, Cl, Br, and CH₃;
ring B is selected from 5- to 6-membered heteroaryl, and the 5- to 6-membered heteroaryl is optionally substituted by 1, 2, or 3 R₁;
R₁ is selected from F, Cl, Br, I, OH, NH₂, CN, CH₃, and OCH₃;
R₂ is selected from

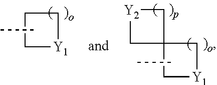

and the

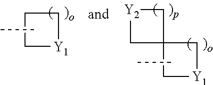

are optionally substituted by 1, 2, or 3 Rₐ;
Y₁ and Y₂ are each independently selected from CH₂, NH, and O;
o and p are each independently selected from 0, 1, 2, and 3;
R₃ is selected from —C(═O)—NH—R_{b}, —C(═O)—R_{b}, —C(═O)—NH—S(═O)₂—R_{b}, —S(═O)₂—NH—R_{b}, —S(═O)₂—R_{b}, —P(═O)(R_{b})₂, C₁₋₃ alkyl, tetrazolyl, isoxazolyl,

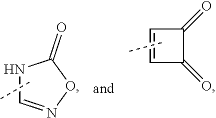

and the C₁₋₃ alkyl, tetrazolyl, isoxazolyl,

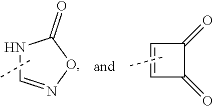

are optionally substituted by 1, 2, or 3 R_{b};
R₄ is selected from F, Cl, Br, I, and C₁₋₃ alkyl;
R₅ is selected from H and CH₃;
n is selected from 0, 1, and 2;
each Rₐ is independently selected from F, Cl, Br, and I;
each R_{b} is independently selected from OH, CN, C₁₋₃ alkyl, C₁₋₃ alkoxy, C₁₋₃ alkylamino, and oxazolyl, and the C₁₋₃ alkyl, C₁₋₃ alkoxy, and oxazolyl are optionally substituted by 1, 2, or 3 R;
each R is independently selected from F, Cl, and Br.

There are still some embodiments of the present disclosure which are obtained by any combination of the above variables.

In some embodiments of the present disclosure, the compound or the pharmaceutically acceptable salt thereof is analyzed by SFC as two independent chromatographic peaks. The SFC analysis method is: column model: Chiralcel OJ-3, 50×4.6 mm I.D., 3 μm, mobile phase A is carbon dioxide, and mobile phase B is selected from methanol (0.1% isopropylamine), ethanol (0.2% ammonia water), or methanol.

In some embodiments of the present disclosure, the proportion of mobile phase B is 5% to 35%, 5% to 50%, or a gradient setting in the SFC analysis method. For example, the gradient setting is that the content of mobile phase B increases from 5% to 50% in 0.2 minutes and holds for 2 minutes, and then decreases from 50% to 5% in 2.2 minutes.

In Example 1 of the present disclosure, compound 001 has a retention time of 1.422 minutes by SFC analysis (chromatographic column: Chiralcel OJ-3, 50×4.6 mm I.D., 3 μm; mobile phase A: supercritical carbon dioxide, B: methanol (0.1% isopropylamine); gradient: the content of B increases from 5% to 50% in 0.2 minutes and holds for 2 minutes, and then decreases from 50% to 5% in 2.2 minutes).

In Example 2 of the present disclosure, compound 002 has a retention time of 1.264 minutes by SFC analysis (chromatographic column: Chiralcel OJ-3, 50×4.6 mm I.D., 3 μm; mobile phase A: supercritical carbon dioxide, B: methanol (0.1% isopropylamine); gradient: the content of B increases from 5% to 50% in 0.2 minutes and holds for 2 minutes, and then decreases from 50% to 5% in 2.2 minutes).

In some examples of the present disclosure, compounds 002 and 002′ were analyzed by chiral HPLC (chromatographic column: Chiralpak IG-U, 50×3.0 mm I.D., 1.6 μm; mobile phase A: n-hexane, B: ethanol (0.1% trifluoroacetic acid); gradient: mobile phase A:B=85:15), showing that compound 002 has a retention time of 2.931 minutes; compound 002′ has a retention time of 4.354 minutes.

In Example 3 of the present disclosure, compound 003 has a retention time of 1.856 minutes by SFC analysis (chromatographic column: Chiralcel OJ-3, 50×4.6 mm I.D., 3 μm; mobile phase A: supercritical carbon dioxide, B: methanol (0.1% isopropylamine); gradient: the content of B increases from 5% to 50% in 0.2 minutes and holds for 2 minutes, and then decreases from 50% to 5% in 2.2 minutes).

In Example 8 of the present disclosure, compound 008 has a retention time of 1.215 minutes by SFC analysis (chromatographic column: Chiralcel OJ-3, 50×4.6 mm I.D., 3 m; mobile phase: A: carbon dioxide, B: methanol (0.1% isopropylamine); gradient: (B %): 5% to 50%)).

In Example 9 of the present disclosure, compound 009 has a retention time of 1.373 minutes by SFC analysis (chromatographic column: Chiralcel OJ-3, 50×4.6 mm I.D., 3 m; mobile phase: A: carbon dioxide, B: methanol (0.1% isopropanol); gradient: (B %): 5% to 50%)).

In Example 10 of the present disclosure, compound 010 has a retention time of 1.264 minutes by SFC analysis (chromatographic column: Chiralpak AD-3, 50×4.6 mm I.D., 3 m; mobile phase: phase A was supercritical carbon dioxide, phase B was methanol (0.1% isopropanol); gradient: the content of B increases from 5% to 50% in 0.2 minutes and holds for 2 minutes, and then decreases from 50% to 5% in 2.2 minutes).

In Example 12 of the present disclosure, compound 012 has a retention time of 1.142 minutes by SFC analysis (chromatographic column: Chiralpak AD-3, 50×4.6 mm I.D., 3 m; mobile phase: A: carbon dioxide, B: ethanol (0.1% isopropanol); gradient: the content of B increases from 5% to 50% in 0.2 minutes and holds for 2 minutes, and then decreases from 50% to 5% in 2.2 minutes).

The present disclosure also provides a compound of the following formula or a pharmaceutically acceptable salt thereof,

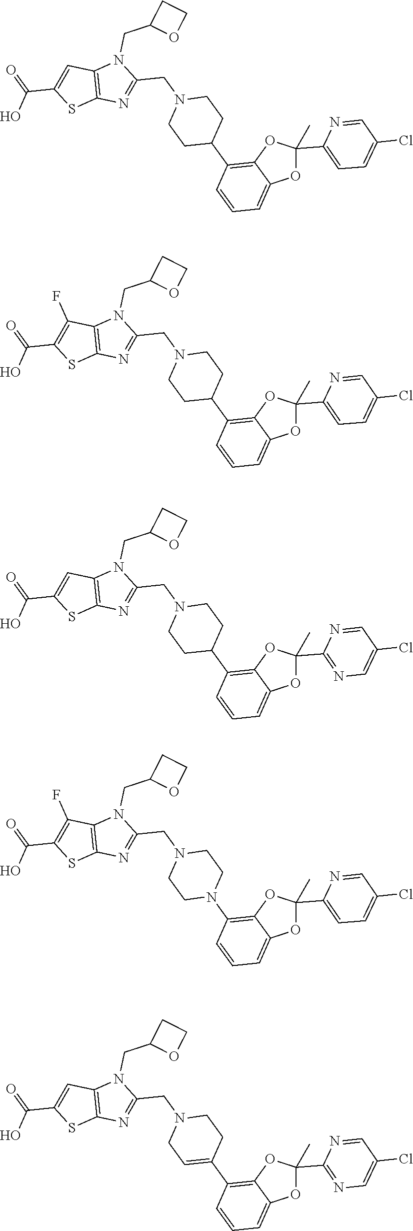 [SMILES: CC1(c2ccc(Cl)cn2)Oc2cccc(C3CCN(Cc4nc5sc(C(=O)O)cc5n4CC4CCO4)CC3)c2O1 ; CC1(c2ccc(Cl)cn2)Oc2cccc(C3CCN(Cc4nc5sc(C(=O)O)c(F)c5n4CC4CCO4)CC3)c2O1 ; CC1(c2ncc(Cl)cn2)Oc2cccc(C3CCN(Cc4nc5sc(C(=O)O)cc5n4CC4CCO4)CC3)c2O1 ; CC1(c2ccc(Cl)cn2)Oc2cccc(N3CCN(Cc4nc5sc(C(=O)O)c(F)c5n4CC4CCO4)CC3)c2O1 ; CC1(c2ncc(Cl)cn2)Oc2cccc(C3=CCN(Cc4nc5sc(C(=O)O)cc5n4CC4CCO4)CC3)c2O1]

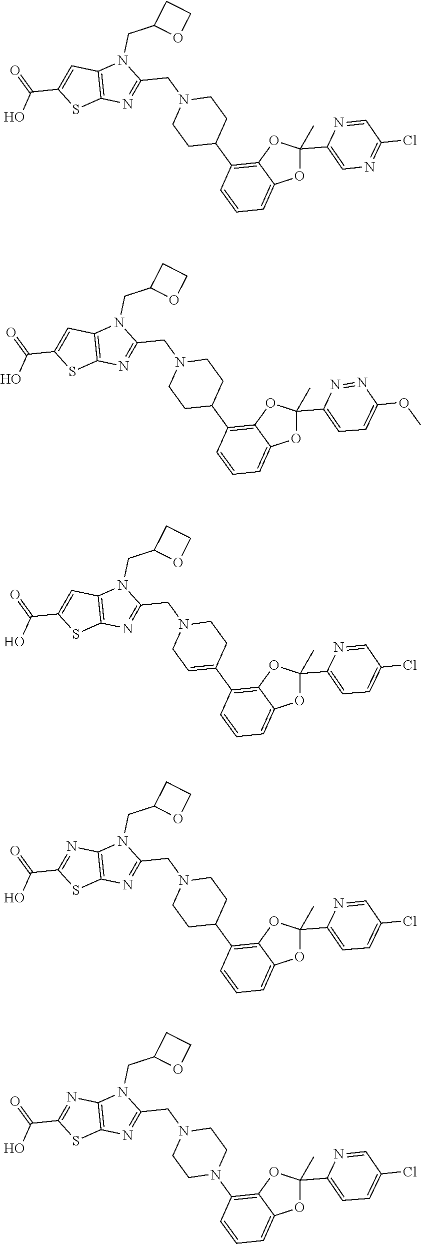 [SMILES: CC1(c2cnc(Cl)cn2)Oc2cccc(C3CCN(Cc4nc5sc(C(=O)O)cc5n4CC4CCO4)CC3)c2O1 ; COc1ccc(C2(C)Oc3cccc(C4CCN(Cc5nc6sc(C(=O)O)cc6n5CC5CCO5)CC4)c3O2)nn1 ; CC1(c2ccc(Cl)cn2)Oc2cccc(C3=CCN(Cc4nc5sc(C(=O)O)cc5n4CC4CCO4)CC3)c2O1 ; CC1(c2ccc(Cl)cn2)Oc2cccc(C3CCN(Cc4nc5sc(C(=O)O)nc5n4CC4CCO4)CC3)c2O1 ; CC1(c2ccc(Cl)cn2)Oc2cccc(N3CCN(Cc4nc5sc(C(=O)O)nc5n4CC4CCO4)CC3)c2O1]

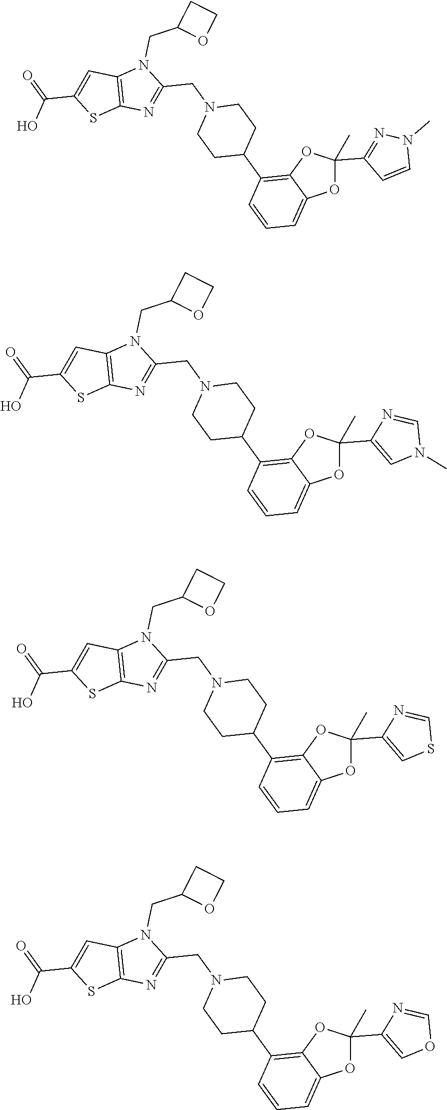 [SMILES: Cn1ccc(C2(C)Oc3cccc(C4CCN(Cc5nc6sc(C(=O)O)cc6n5CC5CCO5)CC4)c3O2)n1 ; Cn1cnc(C2(C)Oc3cccc(C4CCN(Cc5nc6sc(C(=O)O)cc6n5CC5CCO5)CC4)c3O2)c1 ; CC1(c2cscn2)Oc2cccc(C3CCN(Cc4nc5sc(C(=O)O)cc5n4CC4CCO4)CC3)c2O1 ; CC1(c2cocn2)Oc2cccc(C3CCN(Cc4nc5sc(C(=O)O)cc5n4CC4CCO4)CC3)c2O1]

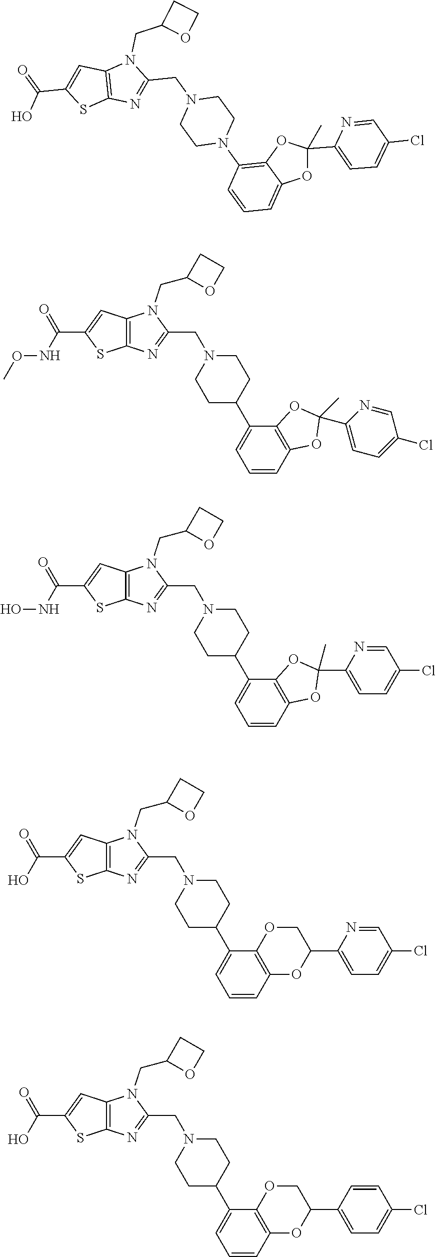 [SMILES: CC1(c2ccc(Cl)cn2)Oc2cccc(N3CCN(Cc4nc5sc(C(=O)O)cc5n4CC4CCO4)CC3)c2O1 ; CONC(=O)c1cc2c(nc(CN3CCC(c4cccc5c4OC(C)(c4ccc(Cl)cn4)O5)CC3)n2CC2CCO2)s1 ; CC1(c2ccc(Cl)cn2)Oc2cccc(C3CCN(Cc4nc5sc(C(=O)NO)cc5n4CC4CCO4)CC3)c2O1 ; O=C(O)c1cc2c(nc(CN3CCC(c4cccc5c4OCC(c4ccc(Cl)cn4)O5)CC3)n2CC2CCO2)s1 ; O=C(O)c1cc2c(nc(CN3CCC(c4cccc5c4OCC(c4ccc(Cl)cc4)O5)CC3)n2CC2CCO2)s1]

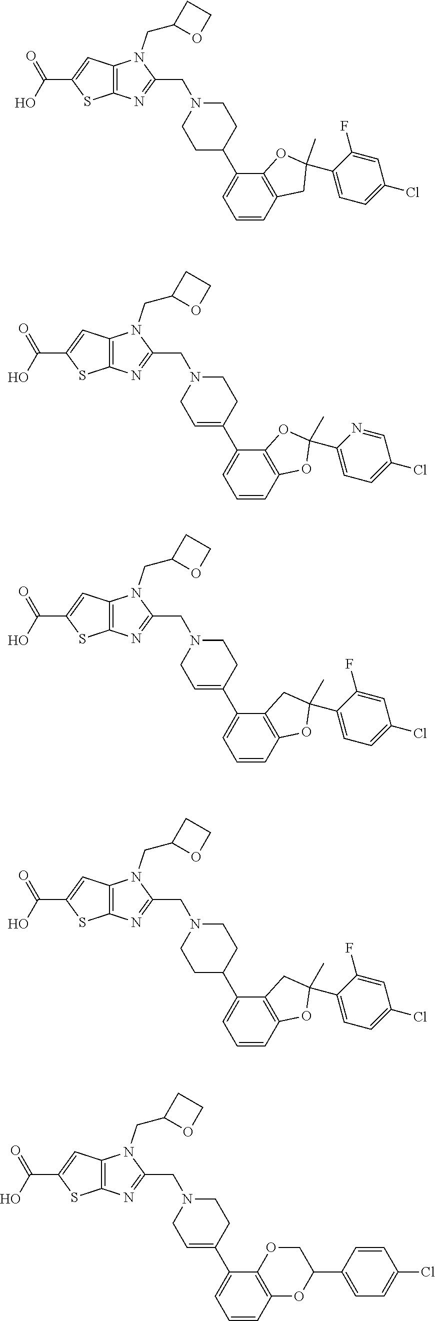 [SMILES: CC1(c2ccc(Cl)cc2F)Cc2cccc(C3CCN(Cc4nc5sc(C(=O)O)cc5n4CC4CCO4)CC3)c2O1 ; CC1(c2ccc(Cl)cn2)Oc2cccc(C3=CCN(Cc4nc5sc(C(=O)O)cc5n4CC4CCO4)CC3)c2O1 ; CC1(c2ccc(Cl)cc2F)Cc2c(cccc2C2=CCN(Cc3nc4sc(C(=O)O)cc4n3CC3CCO3)CC2)O1 ; CC1(c2ccc(Cl)cc2F)Cc2c(cccc2C2CCN(Cc3nc4sc(C(=O)O)cc4n3CC3CCO3)CC2)O1 ; O=C(O)c1cc2c(nc(CN3CC=C(c4cccc5c4OCC(c4ccc(Cl)cc4)O5)CC3)n2CC2CCO2)s1]

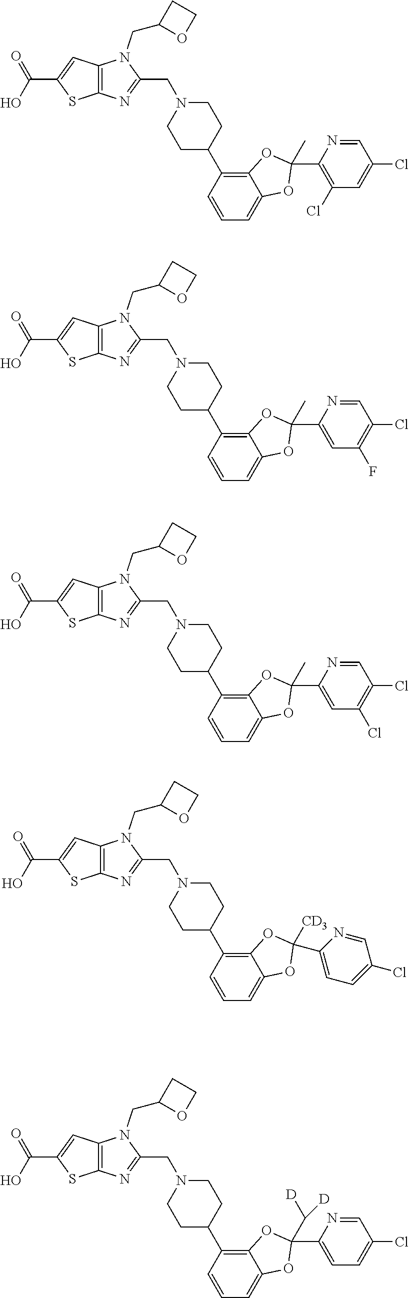 [SMILES: CC1(c2ncc(Cl)cc2Cl)Oc2cccc(C3CCN(Cc4nc5sc(C(=O)O)cc5n4CC4CCO4)CC3)c2O1 ; CC1(c2cc(F)c(Cl)cn2)Oc2cccc(C3CCN(Cc4nc5sc(C(=O)O)cc5n4CC4CCO4)CC3)c2O1 ; CC1(c2cc(Cl)c(Cl)cn2)Oc2cccc(C3CCN(Cc4nc5sc(C(=O)O)cc5n4CC4CCO4)CC3)c2O1 ; [2H]C([2H])([2H])C1(c2ccc(Cl)cn2)Oc2cccc(C3CCN(Cc4nc5sc(C(=O)O)cc5n4CC4CCO4)CC3)c2O1 ; [2H]C([2H])C1(c2ccc(Cl)cn2)Oc2cccc(C3CCN(Cc4nc5sc(C(=O)O)cc5n4CC4CCO4)CC3)c2O1]

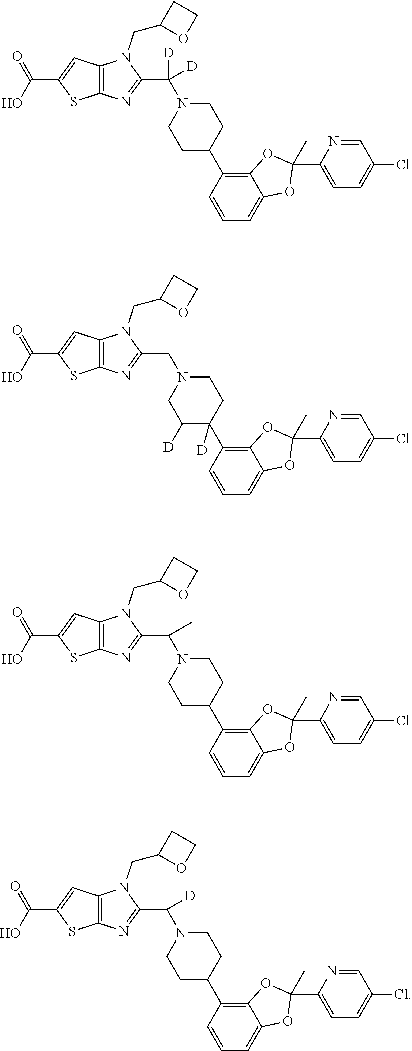 [SMILES: [2H]C([2H])(c1nc2sc(C(=O)O)cc2n1CC1CCO1)N1CCC(c2cccc3c2OC(C)(c2ccc(Cl)cn2)O3)CC1 ; [2H]C1CN(Cc2nc3sc(C(=O)O)cc3n2CC2CCO2)CCC1([2H])c1cccc2c1OC(C)(c1ccc(Cl)cn1)O2 ; CC(c1nc2sc(C(=O)O)cc2n1CC1CCO1)N1CCC(c2cccc3c2OC(C)(c2ccc(Cl)cn2)O3)CC1 ; [2H]C(c1nc2sc(C(=O)O)cc2n1CC1CCO1)N1CCC(c2cccc3c2OC(C)(c2ccc(Cl)cn2)O3)CC1]

In some embodiments of the present disclosure, the compound or the pharmaceutically acceptable salt thereof is selected from:

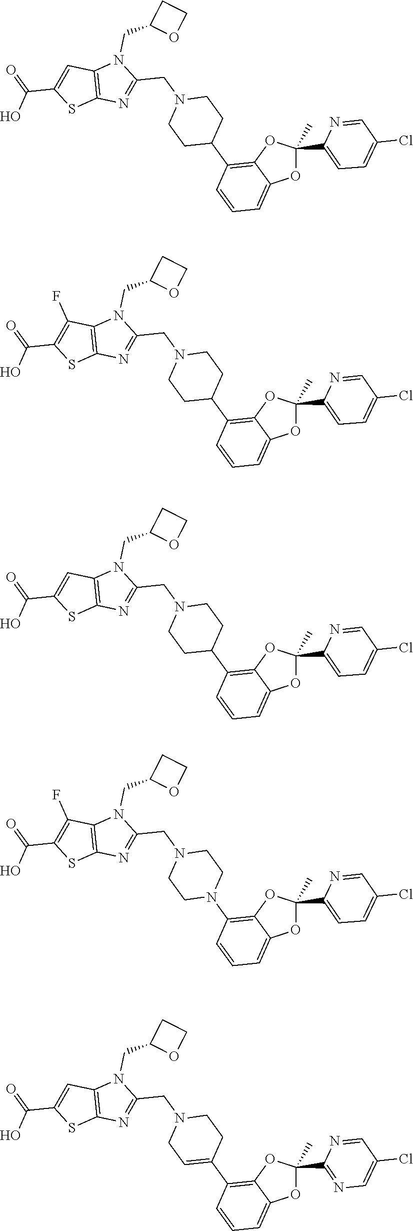 [SMILES: C[C@]1(c2ccc(Cl)cn2)Oc2cccc(C3CCN(Cc4nc5sc(C(=O)O)cc5n4C[C@@H]4CCO4)CC3)c2O1 ; C[C@]1(c2ccc(Cl)cn2)Oc2cccc(C3CCN(Cc4nc5sc(C(=O)O)c(F)c5n4C[C@@H]4CCO4)CC3)c2O1 ; C[C@]1(c2ccc(Cl)cn2)Oc2cccc(C3CCN(Cc4nc5sc(C(=O)O)cc5n4C[C@@H]4CCO4)CC3)c2O1 ; C[C@]1(c2ccc(Cl)cn2)Oc2cccc(N3CCN(Cc4nc5sc(C(=O)O)c(F)c5n4C[C@@H]4CCO4)CC3)c2O1 ; C[C@]1(c2ncc(Cl)cn2)Oc2cccc(C3=CCN(Cc4nc5sc(C(=O)O)cc5n4C[C@@H]4CCO4)CC3)c2O1]

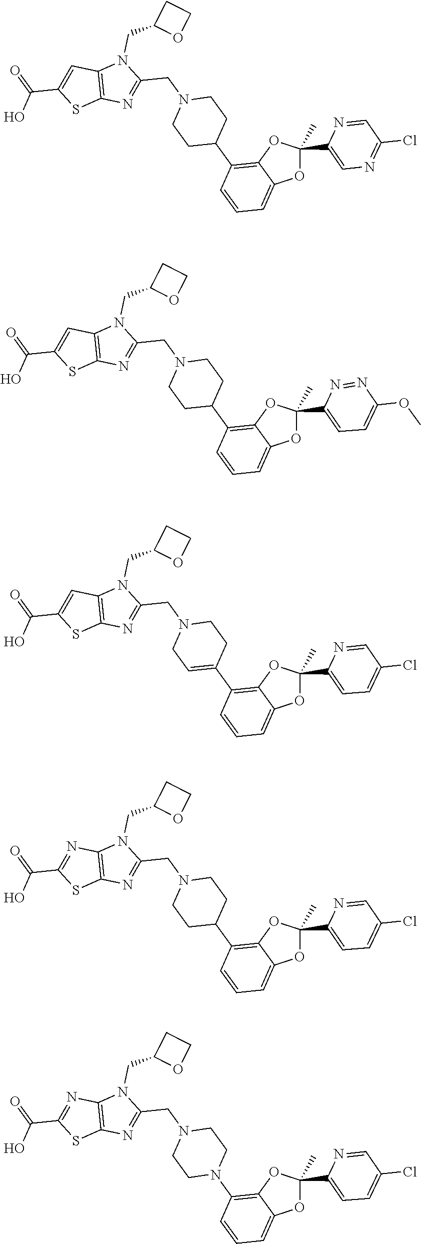 [SMILES: C[C@]1(c2cnc(Cl)cn2)Oc2cccc(C3CCN(Cc4nc5sc(C(=O)O)cc5n4C[C@@H]4CCO4)CC3)c2O1 ; COc1ccc([C@@]2(C)Oc3cccc(C4CCN(Cc5nc6sc(C(=O)O)cc6n5C[C@@H]5CCO5)CC4)c3O2)nn1 ; C[C@]1(c2ccc(Cl)cn2)Oc2cccc(C3=CCN(Cc4nc5sc(C(=O)O)cc5n4C[C@@H]4CCO4)CC3)c2O1 ; C[C@]1(c2ccc(Cl)cn2)Oc2cccc(C3CCN(Cc4nc5sc(C(=O)O)nc5n4C[C@@H]4CCO4)CC3)c2O1 ; C[C@]1(c2ccc(Cl)cn2)Oc2cccc(N3CCN(Cc4nc5sc(C(=O)O)nc5n4C[C@@H]4CCO4)CC3)c2O1]

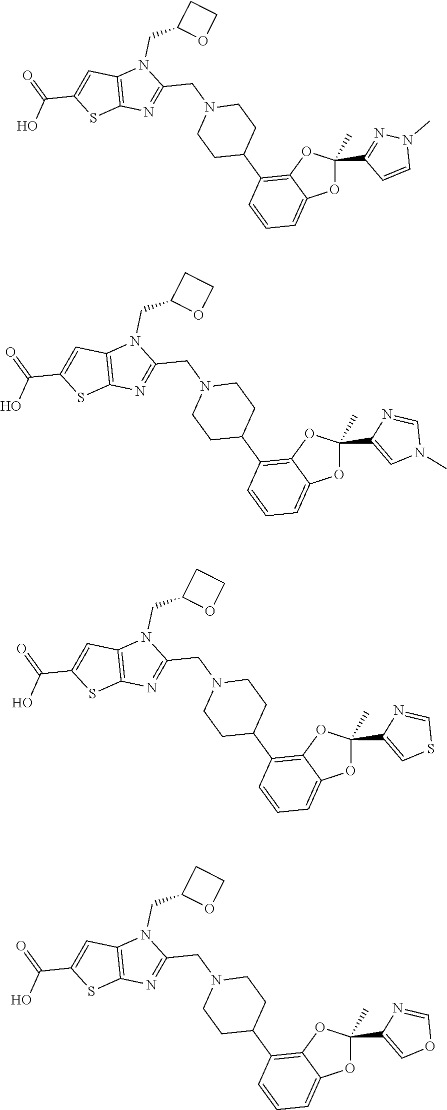 [SMILES: Cn1ccc([C@@]2(C)Oc3cccc(C4CCN(Cc5nc6sc(C(=O)O)cc6n5C[C@@H]5CCO5)CC4)c3O2)n1 ; Cn1cnc([C@@]2(C)Oc3cccc(C4CCN(Cc5nc6sc(C(=O)O)cc6n5C[C@@H]5CCO5)CC4)c3O2)c1 ; C[C@]1(c2cscn2)Oc2cccc(C3CCN(Cc4nc5sc(C(=O)O)cc5n4C[C@@H]4CCO4)CC3)c2O1 ; C[C@]1(c2cocn2)Oc2cccc(C3CCN(Cc4nc5sc(C(=O)O)cc5n4C[C@@H]4CCO4)CC3)c2O1]

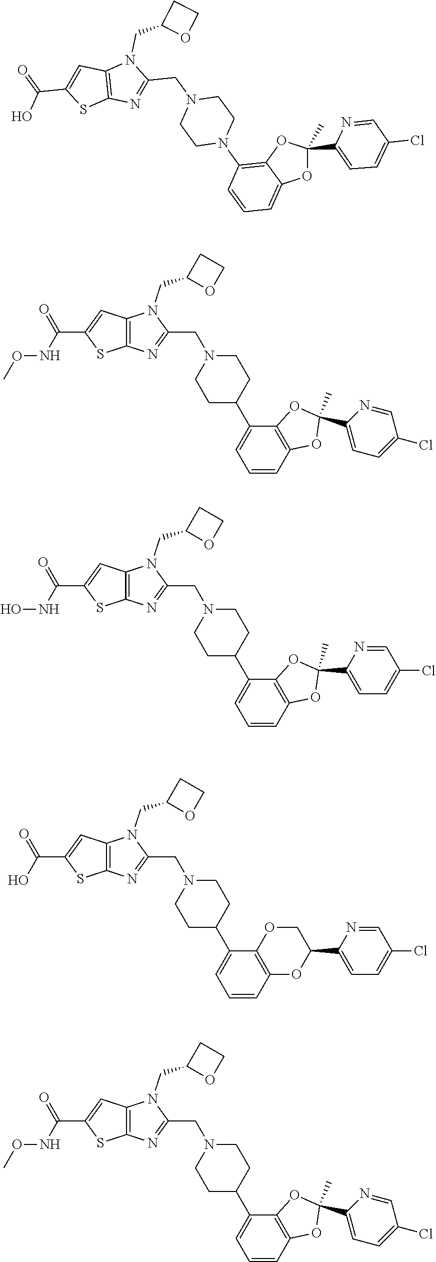 [SMILES: C[C@]1(c2ccc(Cl)cn2)Oc2cccc(N3CCN(Cc4nc5sc(C(=O)O)cc5n4C[C@@H]4CCO4)CC3)c2O1 ; CONC(=O)c1cc2c(nc(CN3CCC(c4cccc5c4O[C@@](C)(c4ccc(Cl)cn4)O5)CC3)n2C[C@@H]2CCO2)s1 ; C[C@]1(c2ccc(Cl)cn2)Oc2cccc(C3CCN(Cc4nc5sc(C(=O)NO)cc5n4C[C@@H]4CCO4)CC3)c2O1 ; O=C(O)c1cc2c(nc(CN3CCC(c4cccc5c4OC[C@@H](c4ccc(Cl)cn4)O5)CC3)n2C[C@@H]2CCO2)s1 ; CONC(=O)c1cc2c(nc(CN3CCC(c4cccc5c4O[C@@](C)(c4ccc(Cl)cn4)O5)CC3)n2C[C@@H]2CCO2)s1]

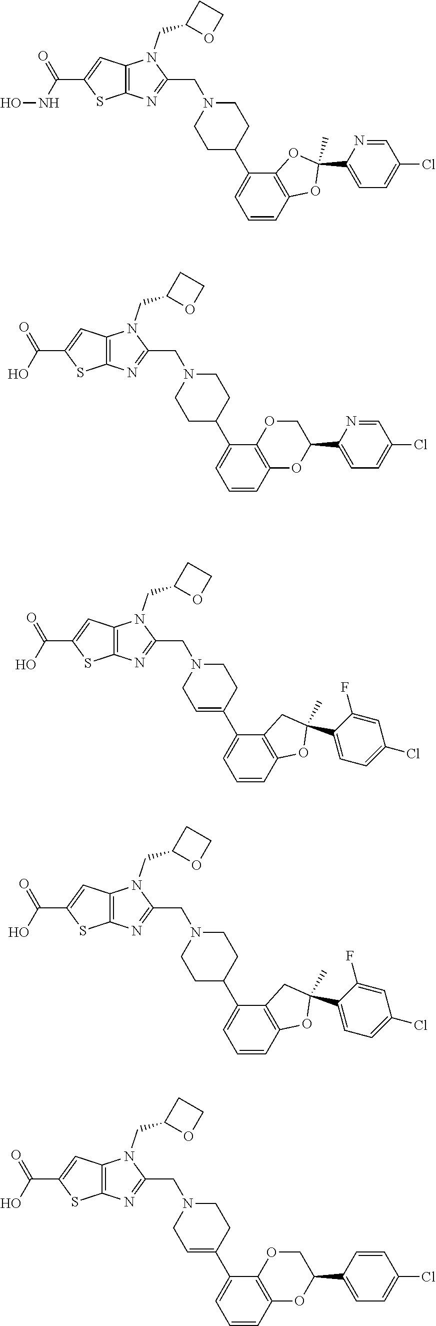 [SMILES: C[C@]1(c2ccc(Cl)cn2)Oc2cccc(C3CCN(Cc4nc5sc(C(=O)NO)cc5n4C[C@@H]4CCO4)CC3)c2O1 ; O=C(O)c1cc2c(nc(CN3CCC(c4cccc5c4OC[C@@H](c4ccc(Cl)cn4)O5)CC3)n2C[C@@H]2CCO2)s1 ; C[C@@]1(c2ccc(Cl)cc2F)Cc2c(cccc2C2=CCN(Cc3nc4sc(C(=O)O)cc4n3C[C@@H]3CCO3)CC2)O1 ; C[C@@]1(c2ccc(Cl)cc2F)Cc2c(cccc2C2CCN(Cc3nc4sc(C(=O)O)cc4n3C[C@@H]3CCO3)CC2)O1 ; O=C(O)c1cc2c(nc(CN3CC=C(c4cccc5c4OC[C@@H](c4ccc(Cl)cc4)O5)CC3)n2C[C@@H]2CCO2)s1]

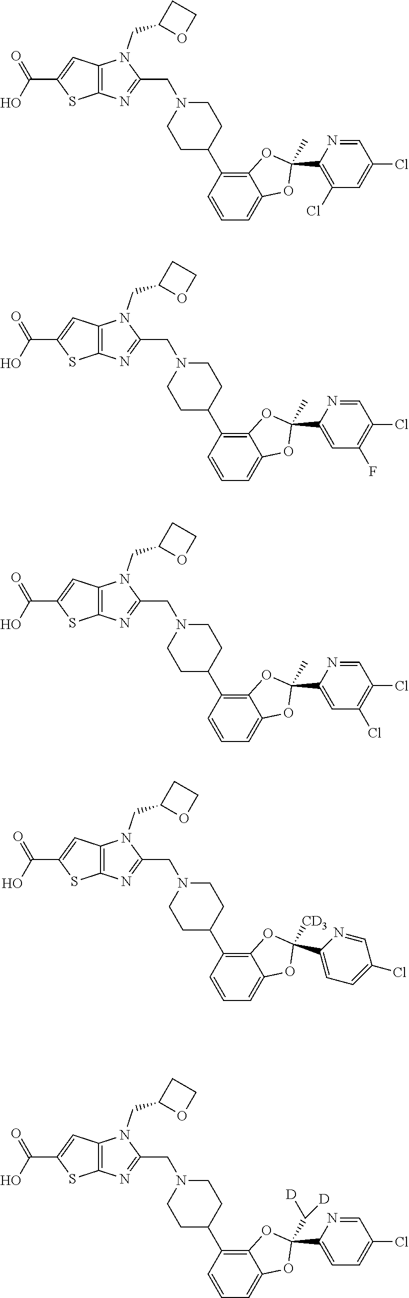 [SMILES: C[C@]1(c2ncc(Cl)cc2Cl)Oc2cccc(C3CCN(Cc4nc5sc(C(=O)O)cc5n4C[C@@H]4CCO4)CC3)c2O1 ; C[C@]1(c2cc(F)c(Cl)cn2)Oc2cccc(C3CCN(Cc4nc5sc(C(=O)O)cc5n4C[C@@H]4CCO4)CC3)c2O1 ; C[C@]1(c2cc(Cl)c(Cl)cn2)Oc2cccc(C3CCN(Cc4nc5sc(C(=O)O)cc5n4C[C@@H]4CCO4)CC3)c2O1 ; [2H]C([2H])([2H])[C@]1(c2ccc(Cl)cn2)Oc2cccc(C3CCN(Cc4nc5sc(C(=O)O)cc5n4C[C@@H]4CCO4)CC3)c2O1 ; [2H]C([2H])[C@]1(c2ccc(Cl)cn2)Oc2cccc(C3CCN(Cc4nc5sc(C(=O)O)cc5n4C[C@@H]4CCO4)CC3)c2O1]

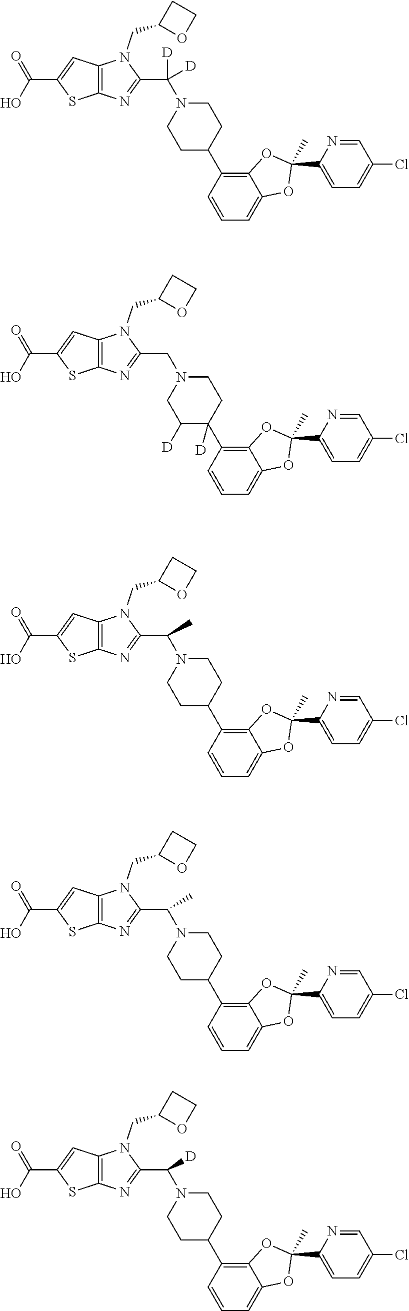 [SMILES: [2H]C([2H])(c1nc2sc(C(=O)O)cc2n1C[C@@H]1CCO1)N1CCC(c2cccc3c2O[C@@](C)(c2ccc(Cl)cn2)O3)CC1 ; [2H]C1CN(Cc2nc3sc(C(=O)O)cc3n2C[C@@H]2CCO2)CCC1([2H])c1cccc2c1O[C@@](C)(c1ccc(Cl)cn1)O2 ; C[C@H](c1nc2sc(C(=O)O)cc2n1C[C@@H]1CCO1)N1CCC(c2cccc3c2O[C@@](C)(c2ccc(Cl)cn2)O3)CC1 ; C[C@@H](c1nc2sc(C(=O)O)cc2n1C[C@@H]1CCO1)N1CCC(c2cccc3c2O[C@@](C)(c2ccc(Cl)cn2)O3)CC1 ; [2H][C@H](c1nc2sc(C(=O)O)cc2n1C[C@@H]1CCO1)N1CCC(c2cccc3c2O[C@@](C)(c2ccc(Cl)cn2)O3)CC1]

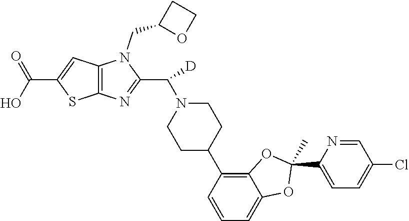 [SMILES: [2H][C@@H](c1nc2sc(C(=O)O)cc2n1C[C@@H]1CCO1)N1CCC(c2cccc3c2O[C@@](C)(c2ccc(Cl)cn2)O3)CC1]

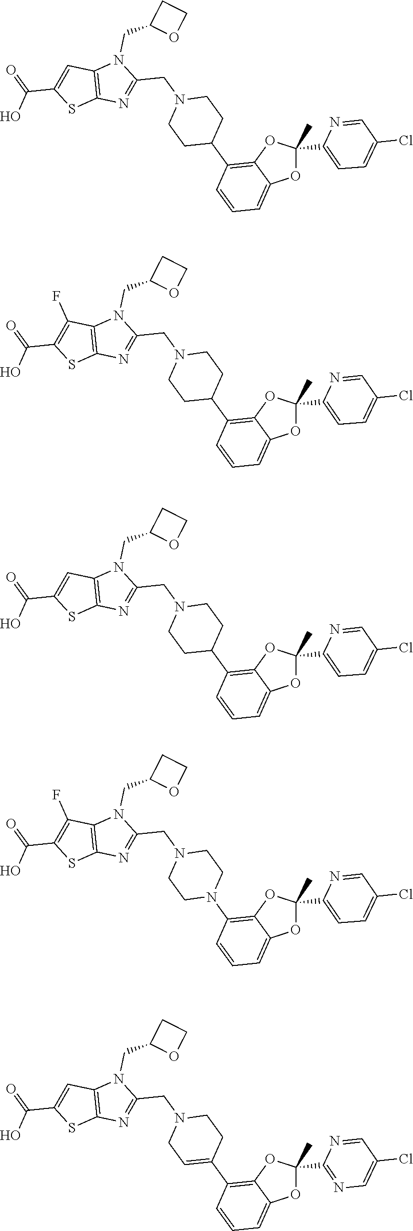 [SMILES: C[C@@]1(c2ccc(Cl)cn2)Oc2cccc(C3CCN(Cc4nc5sc(C(=O)O)cc5n4C[C@@H]4CCO4)CC3)c2O1 ; C[C@@]1(c2ccc(Cl)cn2)Oc2cccc(C3CCN(Cc4nc5sc(C(=O)O)c(F)c5n4C[C@@H]4CCO4)CC3)c2O1 ; C[C@@]1(c2ccc(Cl)cn2)Oc2cccc(C3CCN(Cc4nc5sc(C(=O)O)cc5n4C[C@@H]4CCO4)CC3)c2O1 ; C[C@@]1(c2ccc(Cl)cn2)Oc2cccc(N3CCN(Cc4nc5sc(C(=O)O)c(F)c5n4C[C@@H]4CCO4)CC3)c2O1 ; C[C@@]1(c2ncc(Cl)cn2)Oc2cccc(C3=CCN(Cc4nc5sc(C(=O)O)cc5n4C[C@@H]4CCO4)CC3)c2O1]

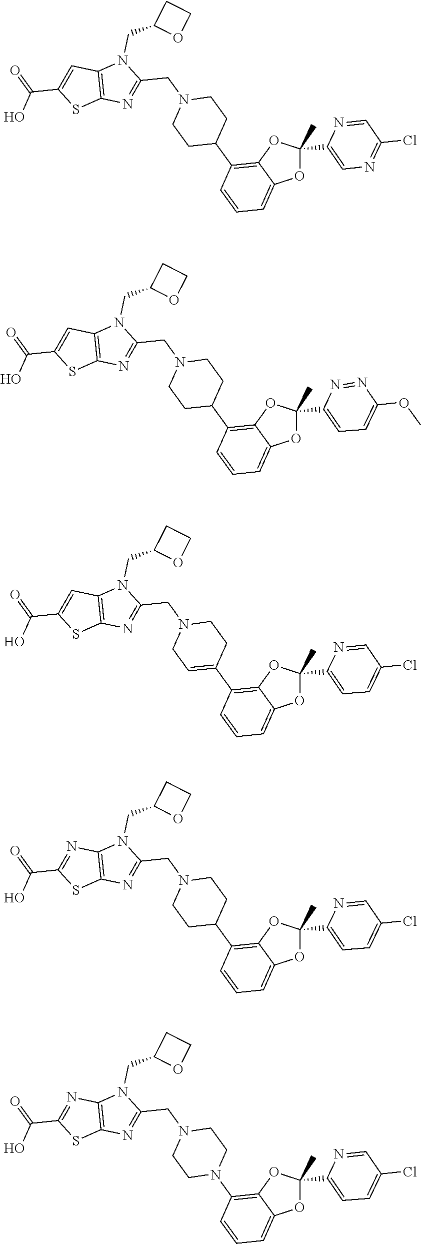 [SMILES: C[C@@]1(c2cnc(Cl)cn2)Oc2cccc(C3CCN(Cc4nc5sc(C(=O)O)cc5n4C[C@@H]4CCO4)CC3)c2O1 ; COc1ccc([C@]2(C)Oc3cccc(C4CCN(Cc5nc6sc(C(=O)O)cc6n5C[C@@H]5CCO5)CC4)c3O2)nn1 ; C[C@@]1(c2ccc(Cl)cn2)Oc2cccc(C3=CCN(Cc4nc5sc(C(=O)O)cc5n4C[C@@H]4CCO4)CC3)c2O1 ; C[C@@]1(c2ccc(Cl)cn2)Oc2cccc(C3CCN(Cc4nc5sc(C(=O)O)nc5n4C[C@@H]4CCO4)CC3)c2O1 ; C[C@@]1(c2ccc(Cl)cn2)Oc2cccc(N3CCN(Cc4nc5sc(C(=O)O)nc5n4C[C@@H]4CCO4)CC3)c2O1]

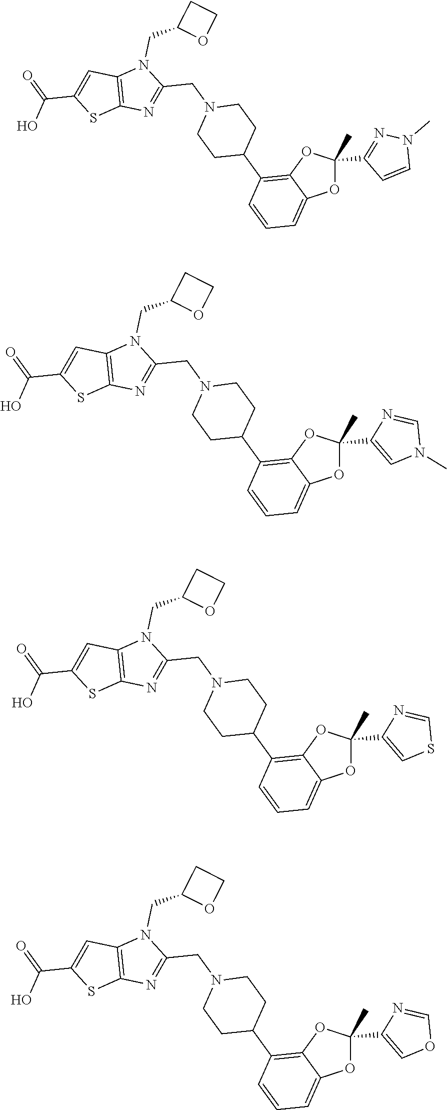 [SMILES: Cn1ccc([C@]2(C)Oc3cccc(C4CCN(Cc5nc6sc(C(=O)O)cc6n5C[C@@H]5CCO5)CC4)c3O2)n1 ; Cn1cnc([C@]2(C)Oc3cccc(C4CCN(Cc5nc6sc(C(=O)O)cc6n5C[C@@H]5CCO5)CC4)c3O2)c1 ; C[C@@]1(c2cscn2)Oc2cccc(C3CCN(Cc4nc5sc(C(=O)O)cc5n4C[C@@H]4CCO4)CC3)c2O1 ; C[C@@]1(c2cocn2)Oc2cccc(C3CCN(Cc4nc5sc(C(=O)O)cc5n4C[C@@H]4CCO4)CC3)c2O1]

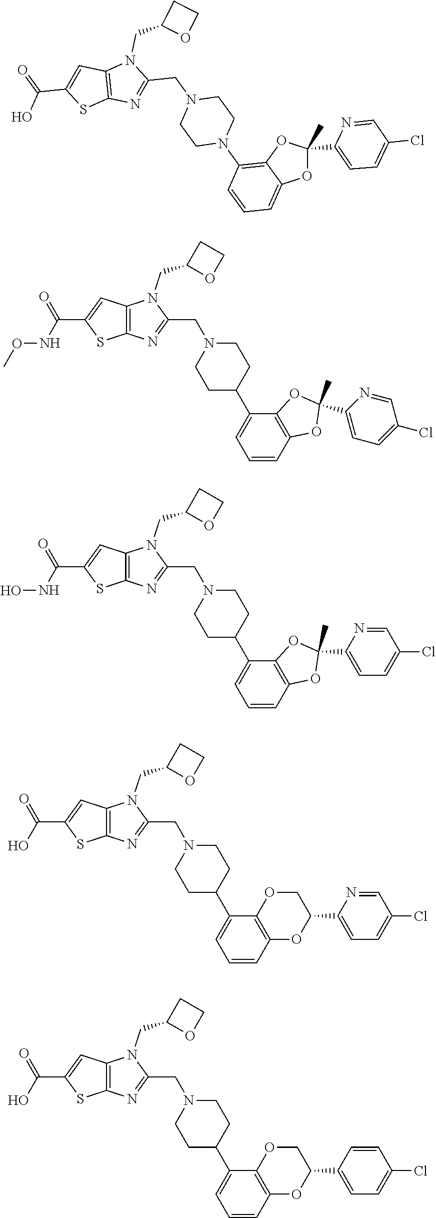 [SMILES: C[C@@]1(c2ccc(Cl)cn2)Oc2cccc(N3CCN(Cc4nc5sc(C(=O)O)cc5n4C[C@@H]4CCO4)CC3)c2O1 ; CONC(=O)c1cc2c(nc(CN3CCC(c4cccc5c4O[C@](C)(c4ccc(Cl)cn4)O5)CC3)n2C[C@@H]2CCO2)s1 ; C[C@@]1(c2ccc(Cl)cn2)Oc2cccc(C3CCN(Cc4nc5sc(C(=O)NO)cc5n4C[C@@H]4CCO4)CC3)c2O1 ; O=C(O)c1cc2c(nc(CN3CCC(c4cccc5c4OC[C@H](c4ccc(Cl)cn4)O5)CC3)n2C[C@@H]2CCO2)s1 ; O=C(O)c1cc2c(nc(CN3CCC(c4cccc5c4OC[C@H](c4ccc(Cl)cc4)O5)CC3)n2C[C@@H]2CCO2)s1]

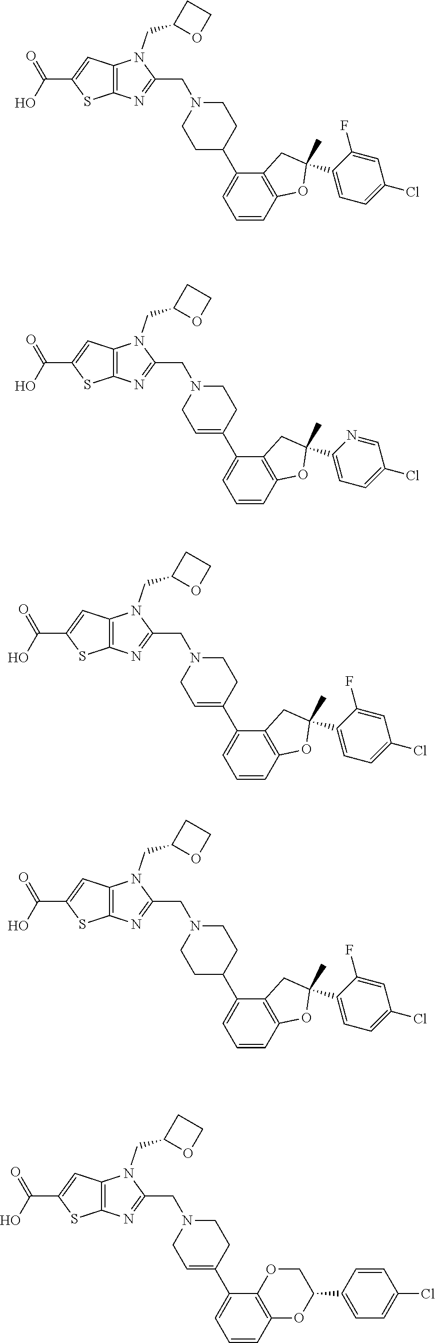 [SMILES: C[C@]1(c2ccc(Cl)cc2F)Cc2c(cccc2C2CCN(Cc3nc4sc(C(=O)O)cc4n3C[C@@H]3CCO3)CC2)O1 ; C[C@]1(c2ccc(Cl)cn2)Cc2c(cccc2C2=CCN(Cc3nc4sc(C(=O)O)cc4n3C[C@@H]3CCO3)CC2)O1 ; C[C@]1(c2ccc(Cl)cc2F)Cc2c(cccc2C2=CCN(Cc3nc4sc(C(=O)O)cc4n3C[C@@H]3CCO3)CC2)O1 ; C[C@]1(c2ccc(Cl)cc2F)Cc2c(cccc2C2CCN(Cc3nc4sc(C(=O)O)cc4n3C[C@@H]3CCO3)CC2)O1 ; O=C(O)c1cc2c(nc(CN3CC=C(c4cccc5c4OC[C@H](c4ccc(Cl)cc4)O5)CC3)n2C[C@@H]2CCO2)s1]

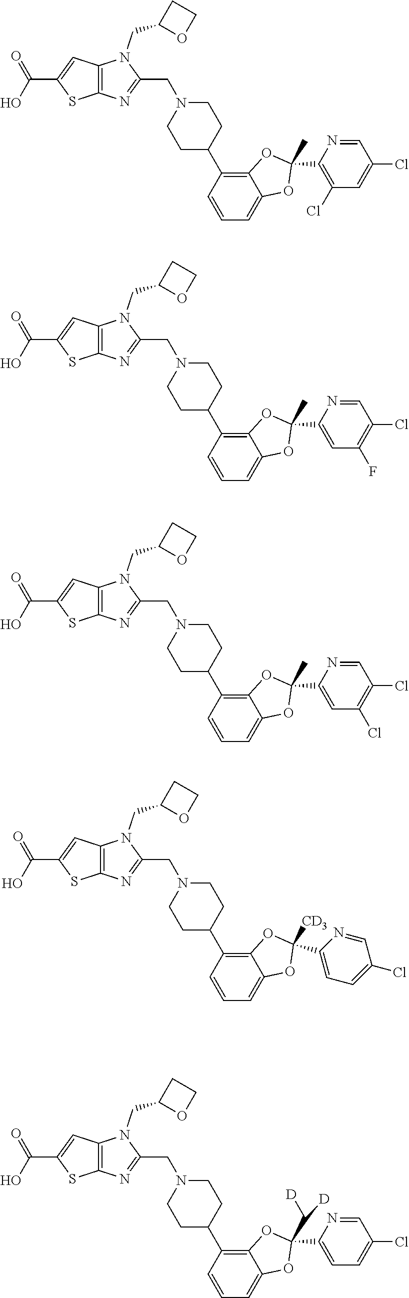 [SMILES: C[C@@]1(c2ncc(Cl)cc2Cl)Oc2cccc(C3CCN(Cc4nc5sc(C(=O)O)cc5n4C[C@@H]4CCO4)CC3)c2O1 ; C[C@@]1(c2cc(F)c(Cl)cn2)Oc2cccc(C3CCN(Cc4nc5sc(C(=O)O)cc5n4C[C@@H]4CCO4)CC3)c2O1 ; C[C@@]1(c2cc(Cl)c(Cl)cn2)Oc2cccc(C3CCN(Cc4nc5sc(C(=O)O)cc5n4C[C@@H]4CCO4)CC3)c2O1 ; [2H]C([2H])([2H])[C@@]1(c2ccc(Cl)cn2)Oc2cccc(C3CCN(Cc4nc5sc(C(=O)O)cc5n4C[C@@H]4CCO4)CC3)c2O1 ; [2H]C([2H])[C@@]1(c2ccc(Cl)cn2)Oc2cccc(C3CCN(Cc4nc5sc(C(=O)O)cc5n4C[C@@H]4CCO4)CC3)c2O1]

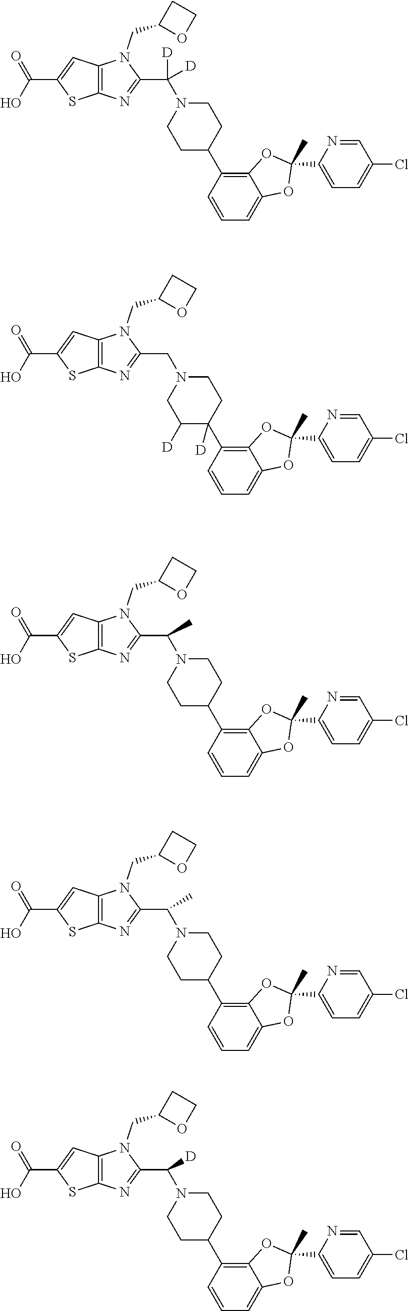 [SMILES: [2H]C([2H])(c1nc2sc(C(=O)O)cc2n1C[C@@H]1CCO1)N1CCC(c2cccc3c2O[C@](C)(c2ccc(Cl)cn2)O3)CC1 ; [2H]C1CN(Cc2nc3sc(C(=O)O)cc3n2C[C@@H]2CCO2)CCC1([2H])c1cccc2c1O[C@](C)(c1ccc(Cl)cn1)O2 ; C[C@H](c1nc2sc(C(=O)O)cc2n1C[C@@H]1CCO1)N1CCC(c2cccc3c2O[C@](C)(c2ccc(Cl)cn2)O3)CC1 ; C[C@@H](c1nc2sc(C(=O)O)cc2n1C[C@@H]1CCO1)N1CCC(c2cccc3c2O[C@](C)(c2ccc(Cl)cn2)O3)CC1 ; [2H][C@H](c1nc2sc(C(=O)O)cc2n1C[C@@H]1CCO1)N1CCC(c2cccc3c2O[C@](C)(c2ccc(Cl)cn2)O3)CC1]

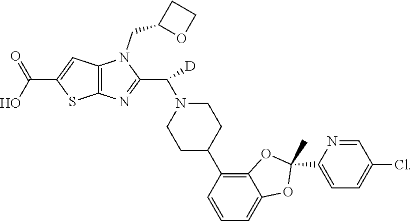 [SMILES: [2H][C@@H](c1nc2sc(C(=O)O)cc2n1C[C@@H]1CCO1)N1CCC(c2cccc3c2O[C@](C)(c2ccc(Cl)cn2)O3)CC1]

The present disclosure further provides a use of the compound or the pharmaceutically acceptable salt thereof in the manufacture of a medicament for treating diabetes.

### Definition and Description

Unless otherwise specified, the following terms and phrases when used herein have the following meanings. A specific term or phrase should not be considered indefinite or unclear in the absence of a particular definition, but should be understood in the ordinary sense. When a trading name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction, or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by contacting such compounds with a sufficient amount of base in a pure solution or a suitable inert solvent. When the compound of the present disclosure contains a relatively basic functional group, an acid addition salt can be obtained by contacting such compounds with a sufficient amount of acid in a pure solution or a suitable inert solvent. Certain specific compounds of the present disclosure contain both basic and acidic functional groups, and thus can be converted to any base or acid addition salt.

The pharmaceutically acceptable salt of the present disclosure can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and (S)-enantiomers, diastereoisomers, (D)-isomers, (L)-isomers, racemic, and other mixtures thereof, such as enantiomers or diastereomer enriched mixtures, all of which are within the scope of the present disclosure. Additional asymmetric carbon atoms may be present in substituents such as alkyl. All these isomers and their mixtures are included within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and or “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) or a straight dashed bond ( [symbol] ).

Unless otherwise specified, when a double bond structure, such as carbon-carbon double bond, carbon-nitrogen double bond, and nitrogen-nitrogen double bond, exists in the compound, and each of the atoms on the double bond is connected to two different substituents (including the condition where a double bond contains a nitrogen atom, the lone pair of electrons attached on the nitrogen atom is regarded as a substituent connected), if the atom on the double bond in the compound and its substituent are represented by [symbol] this refers to the (Z) isomer, (E) isomer or a mixture of two isomers of the compound.

Unless otherwise specified, the term “tautomer” or “tautomeric form” means that at room temperature, the isomers of different functional groups are in dynamic equilibrium and can be transformed into each other quickly. If tautomers possibly exist (such as in solution), the chemical equilibrium of tautomers can be reached. For example, proton tautomer (also called prototropic tautomer) includes interconversion through proton migration, such as keto-enol isomerization and imine-enamine isomerization. Valence tautomer includes some recombination of bonding electrons for mutual transformation. A specific example of keto-enol tautomerization is the tautomerism between two tautomers of pentane-2,4-dione and 4-hydroxypent-3-en-2-one.

Unless otherwise specified, the terms “enriched in one isomer”, “enriched in isomers”, “enriched in one enantiomer” or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (S)-isomers, or D and L isomers can be prepared using chiral synthesis, chiral reagents, or other conventional techniques. If one kind of enantiomer of certain compound of the present disclosure is to be obtained, it can be obtained by asymmetric synthesis or derivative action of chiral auxiliary, wherein the resulting diastereomeric mixture is separated and the auxiliary group is cleaved to provide the pure desired enantiomer. Alternatively, when the molecule contains a basic functional group (such as amino) or an acidic functional group (such as carboxyl), a salt of a diastereoisomer is formed with an appropriate optically active acid or base, and then diastereomeric resolution is performed by conventional methods known in the art, and then the pure enantiomer is recovered. In addition, the enantiomer and the diastereoisomer are generally separated through chromatography which uses a chiral stationary phase and optionally combines with a chemical derivative method (such as carbamate generated from amine).

The compound of the present disclosure may contain an unnatural proportion of atomic isotope at one or more atoms that constitute the compound. For example, the compound can be radiolabeled with a radioactive isotope, such as tritium (³H), iodine-125 (¹²⁵I), or C-14 (¹⁴C). For another example, deuterated drugs can be formed by replacing hydrogen with deuterium, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “optional” or “optionally” means that the subsequent event or condition may occur but not requisite, that the term includes the instance in which the event or condition occurs and the instance in which the event or condition does not occur.

The term “substituted” means one or more than one hydrogen atom on a specific atom is substituted by the substituent, including deuterium and hydrogen variables, as long as the valence of the specific atom is normal and the substituted compound is stable. When the substituent is an oxygen (i.e., ═O), it means two hydrogen atoms are substituted. Positions on an aromatic ring cannot be substituted with a ketone. The term “optionally substituted” means an atom can be substituted with a substituent or not, unless otherwise specified, the type and number of the substituent may be arbitrary as long as being chemically achievable.

When any variable (such as R) occurs in the constitution or structure of the compound more than once, the definition of the variable at each occurrence is independent. Thus, for example, if a group is substituted by 0 to 2 R, the group can be optionally substituted with up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When one of the variables is selected from a single bond, it means that the two groups linked by the single bond are connected directly. For example, when L in A-L-Z represents a single bond, the structure of A-L-Z is actually A-Z.

When a substituent is vacant, it means that the substituent is absent, for example, when X is vacant in A-X, the structure of A-X is actually A. When the enumerative substituent does not indicate by which atom it is linked to the group to be substituted, such substituent can be bonded by any atom thereof. For example, when pyridyl acts as a substituent, it can be linked to the group to be substituted by any carbon atom on the pyridine ring.

When the enumerative linking group does not indicate the direction for linking, the direction for linking is arbitrary, for example, the linking group L contained in

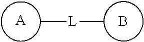

is -M-W—, then -M-W— can link ring A and ring B to form

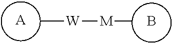

in the direction same as left-to-right reading order, and form

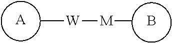

in the direction contrary to left-to-right reading order. A combination of the linking groups, substituents and/or variables thereof is allowed only when such combination can result in a stable compound.

Unless otherwise specified, when a group has one or more linkable sites, any one or more sites of the group can be linked to other groups through chemical bonds. When the linking site of the chemical bond is not positioned, and there is an H atom at the linkable site, then the number of H atoms at the site will decrease correspondingly with the number of the chemical bonds linking thereto so as to meet the corresponding valence. The chemical bond linking the site to other groups can be represented by a straight solid bond ( [symbol] ), straight dashed bond ( [symbol] ), or wave line

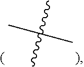

respectively, wherein the straight dashed bond ( [symbol] ) or wave line

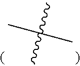

can represent single, double, or triple bonds linked to other groups. Correspondingly, the site will be reduced by 1, 2, or 3H to become a monovalent, bivalent, or trivalent group. For example, the straight solid bond in —OCH₃ means that it is linked to other groups through the oxygen atom in the group; the straight dashed bond in

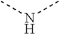

means that it is linked to other groups through the two ends of the nitrogen atom in the group; the wave lines in

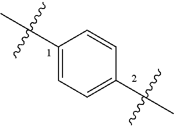 [SMILES: *c1ccc(*)cc1]

means that the phenyl group is linked to other groups through carbon atoms at position 1 and position 2.

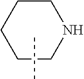

means that it can be linked to other groups through any linkable sites on the piperidinyl by one chemical bond, including at least four types of linkage, including

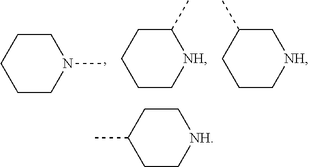

Even though the H atom is drawn on the —N—,

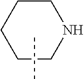

still includes the linkage of

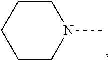

merely when one chemical bond was connected, the H of this site will be reduced by one to the corresponding monovalent piperidinyl.

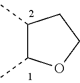

can be linked to other groups through the carbon atoms at positions 1 and 2 of the tetrahydrofuran group, either by a single bond, such as

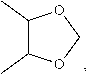

or by a double bond, such as

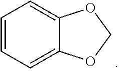

Unless otherwise specified, the term “C₁₋₃ alkyl” refers to a linear or branched saturated hydrocarbon group consisting of 1 to 3 carbon atoms. The C₁₋₃ alkyl includes C₁₋₂ and C₂₋₃ alkyl, etc.; it can be monovalent (such as methyl), divalent (such as methylene), or multivalent (such as methine). Examples of C₁₋₃ alkyl include, but are not limited to, methyl (Me), ethyl (Et), propyl (including n-propyl and isopropyl), etc.

Unless otherwise specified, the term “C₁₋₃ alkoxy” refers to an alkyl group containing 1 to 3 carbon atoms that are connected to the rest of the molecule through an oxygen atom. The C₁₋₃ alkoxy includes C₁₋₂, C₂₋₃, C₃, C₂ alkoxy, etc. Examples of C₁₋₃ alkoxy include, but are not limited to, methoxy, ethoxy, propoxy (including n-propoxy and isopropoxy), etc.

Unless otherwise specified, the term “C₁₋₃ alkylamino” refers to an alkyl group containing 1 to 3 carbon atoms that are connected to the rest of the molecule through an amino group. The C₁₋₃ alkylamino includes C₁₋₂, C₃, C₂ alkylamino, etc. Examples of C₁₋₃ alkylamino include, but are not limited to, —NHCH₃, —N(CH₃)₂, —NHCH₂CH₃, —N(CH₃)CH₂CH₃, —NHCH₂CH₂CH₃, —NHCH₂(CH₃)₂, etc.

Unless otherwise specified, the terms “5- to 6-membered heteroaryl ring” and “5- to 6-membered heteroaryl” are used interchangeably. The term “5- to 6-membered heteroaryl” refers to a monocyclic group consisting of 5 to 6 ring atoms with a conjugated π-electron system, in which 1, 2, 3, or 4 ring atoms are heteroatoms independently selected from O, S, and N, and the rest are carbon atoms, Here, the nitrogen atom is optionally quaternized, and nitrogen and sulfur heteroatoms can be optionally oxidized (i.e., NO and S(O)ₚ, wherein p is 1 or 2). The 5- to 6-membered heteroaryl can be linked to the rest of the molecule through a heteroatom or a carbon atom. The 5- to 6-membered heteroaryl includes 5-membered and 6-membered heteroaryl. Examples of the 5- to 6-membered heteroaryl include, but are not limited to, pyrrolyl (including N-pyrrolyl, 2-pyrrolyl, and 3-pyrrolyl, etc.), pyrazolyl (including 2-pyrazolyl and 3-pyrazolyl, etc.), imidazolyl (including N-imidazolyl, 2-imidazolyl, 4-imidazolyl, and 5-imidazolyl, etc.), oxazolyl (including 2-oxazolyl, 4-oxazolyl, and 5-oxazolyl, etc.), triazolyl (including 1H-1,2,3-triazolyl, 2H-1,2,3-triazolyl, 1H-1,2,4-triazolyl, and 4H-1,2,4-triazolyl, etc.), tetrazolyl, isoxazolyl (including 3-isoxazolyl, 4-isoxazolyl, and 5-isoxazolyl, etc.), thiazolyl (including 2-thiazolyl, 4-thiazolyl, and 5-thiazolyl, etc.), furyl (including 2-furyl and 3-furyl, etc.), thienyl (including 2-thienyl and 3-thienyl, etc.), pyridyl (including 2-pyridyl, 3-pyridyl, and 4-pyridyl, etc.), pyrazinyl, pyrimidinyl (including 2-pyrimidinyl, 4-pyrimidinyl, etc.).

Unless otherwise specified, the term “aromatic ring” means a cyclic group having a conjugated π-electron system, the atoms of which are covered by a delocalized π-electron cloud. In the structural formula, it can be written in the form of alternating single and double bonds, or

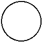

can be used to represent the delocalized π-electron cloud when the rules of atomic valence and covalent bonding are met. For example, the structures represented by the structural formulas

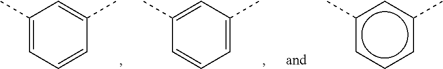

and are all the same; the structures represented by the structural formulas

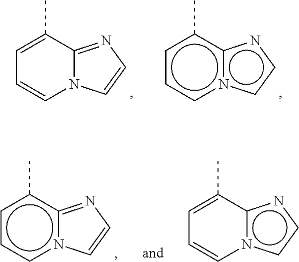

are all the same.

Unless otherwise specified, Cₙ₋ₙ₊ₘ or Cₙ-Cₙ₊ₘ includes any specific case of n to n+m carbons, for example, C₁₋₁₂ includes C₁, C₂, C₃, C₄, C₅, C₆, C₇, C₈, C₉, C₁₀, C₁₁, and C₁₂, and any range from n to n+m is also included, for example C₁₋₁₂ includes C₁₋₃, C₁₋₆, C₁₋₉, C₃-6, C₃-9, C₃₋₁₂, C₆₋₉, C₆₋₁₂, C₉₋₁₂, etc.; similarly, n-membered to n+m-membered means that the number of atoms on the ring is from n to n+m, for example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring, and any range from n to n+m is also included, for example, 3- to 12-membered ring includes 3- to 6-membered ring, 3- to 9-membered ring, 5- to 6-membered ring, 5- to 7-membered ring, 6- to 7-membered ring, 6- to 8-membered ring, 6- to 10-membered ring, etc.

The structure of the compounds of the present disclosure can be confirmed by conventional methods known to those skilled in the art, and if the present disclosure involves an absolute configuration of a compound, then the absolute configuration can be confirmed by means of conventional techniques in the art. For example, in the case of single crystal X-ray diffraction (SXRD), the absolute configuration can be confirmed by collecting diffraction intensity data from the cultured single crystal using a Bruker D8 venture diffractometer with CuKα radiation as the light source and scanning mode: φ/ω scan, and after collecting the relevant data, the crystal structure can be further analyzed by the direct method (Shelxs97).

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include but are not limited to the examples of the present disclosure.

The solvent used in the present disclosure is commercially available. The following abbreviations are used in the present disclosure: aq represents water; eq represents equivalent, equal; DCM represents dichloromethane; PE represents petroleum ether; DMF represents N,N-dimethylformamide; DMSO represents dimethyl sulfoxide; EtOAc and EA both represent ethyl acetate; EtOH represents ethanol; MeOH represents methanol; BOC represents tert-butyloxycarbonyl, which is an amine protecting group; HOAc represents acetic acid; RT and Rt both represent retention time; O/N represents overnight; hr represents hour; THF represents tetrahydrofuran; Boc₂O represents di-tert-butyl dicarbonate; TFA represents trifluoroacetic acid; DIPEA represents diisopropylethylamine; Xantphos represents 4,5-bis(diphenylphosphino)-9,9-dimethyloxanthene; BINAP represents 1,1′-binaphthyl-2,2′-bisdiphenylphosphine; SEMCl represents 2-(trimethylsilyl)ethoxymethyl chloride; TBDPSCl represents tert-butyldiphenylchlorosilane; NBS represents N-bromosuccinimide; i-PrMgCl represents isopropylmagnesium chloride; N₂ represents nitrogen; NaBH₄ represents sodium borohydride; DIAD represents diisopropyl azodicarboxylate; and Pd(PPh₃)₄ represents tetrakis(triphenylphosphine)palladium.

The compounds of the present disclosure are named according to the conventional naming principles in the art or by ChemDraw® software, and the commercially available compounds use the supplier catalog names.

### Technical Effect

The compound of the present disclosure exhibits excellent agonistic activity to GLP-1 receptor, has low risk of time-dependent inhibition of human liver microsomal cytochrome P450 isoenzyme 2C19, and metabolizes slowly in hepatocytes of various species, and show little species difference. The compound of the present disclosure has high oral exposure, long half-life, high bioavailability, and good in vivo pharmacokinetic properties.

### DETAILED DESCRIPTION OF THE PREFERRED EMBODIMENT

The present disclosure is described in detail by the examples below, but it does not mean that there are any adverse restrictions on the present disclosure. The present disclosure has been described in detail herein, and its specific examples have also been disclosed; for one skilled in the art, it is obvious to make various modifications and improvements to the examples of the present disclosure without departing from the spirit and scope of the present disclosure.

### Reference Example 1: Fragment B-1

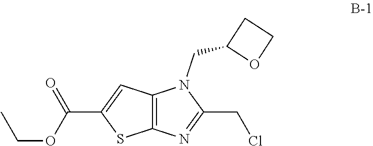 [SMILES: CCOC(=O)c1cc2c(nc(CCl)n2C[C@@H]2CCO2)s1]

### Synthetic Route:

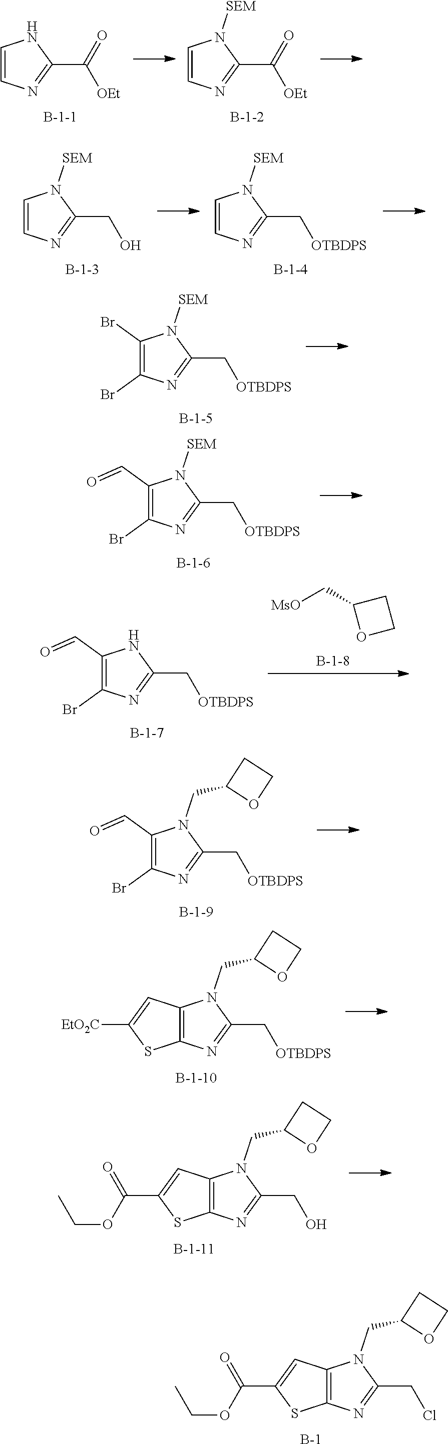

### Step 1: Synthesis of Compound B-1-2

B-1-1 (12.9 g, 92.05 mmol, 1 eq) was dissolved in THF (150.0 mL), then sodium hydride (5.52 g, 138.08 mmol, a content of 60%, 1.5 eq) was slowly added thereto in batches at 0° C. under N₂ atmosphere, and the mixture was stirred to homogeneity, and then SEMCI (23.55 g, 141.25 mmol, 25 mL, 1.53 eq) was added thereto. The reaction system was gradually warmed to 20° C. and stirred for 16 hours. The reaction mixture was slowly poured into ice water (300.0 mL) and stirred thoroughly, extracted with ethyl acetate (150.0 mL*3), and the organic phase was washed with saturated brine (100 mL) and dried over anhydrous sodium sulfate. The resulting mixture was filtered, and the filtrate was concentrated to obtain a crude product, which was purified by silica gel column chromatography (petroleum ether:ethyl acetate=1:0 to 1:1) to obtain compound B-1-2. LCMS: m/z=271.1 [M+1]⁺.

### Step 2: Synthesis of Compound B-1-3

Lithium aluminium hydride (712.91 mg, 18.78 mmol, 1.5 eq) was dissolved in THE (60 mL) and stirred thoroughly, and then a solution of B-1-2 (3.39 g, 12.52 mmol, 1 eq) in THF (10 mL) was slowly added thereto at 0° C. under N₂ atmosphere. The reaction mixture was warmed to 20° C. and stirred for 45 minutes. The reaction mixture was cooled to 0° C., then 1 mL of water and 1 mL of 15% sodium hydroxide solution were sequentially added thereto, and the reaction mixture was warmed to 20° C. and stirred for 15 minutes, then added with a small amount of anhydrous magnesium sulfate, stirred for another 15 minutes, and filtered. The resulting filtrate was washed with saturated brine (350 mL), the aqueous phase was extracted with ethyl acetate (50 mL*3), the resulting organic phase was washed with saturated brine (50 mL), dried over anhydrous sodium sulfate, filtered, and the resulting filtrate was concentrated under reduced pressure to obtain B-1-3. ¹H NMR (400 MHz, CDCl₃) δ ppm 6.95-7.00 (m, 1H), 6.92 (s, 1H), 5.37 (s, 2H), 4.71 (s, 2H), 3.49-3.56 (m, 2H), 0.88-0.94 (m, 2H), 0.02-0.05 (m, 9H). LCMS: m/z=229.1 [M+1]⁺.

### Step 3: Synthesis of Compound B-1-4

B-1-3 (2.4 g, 10.51 mmol, 1 eq) was dissolved in DMF (30 mL) at 20° C. and stirred thoroughly, and then TBDPSCl (1.3 g, 13.63 mmol, 1.3 eq) and imidazole (1.8 g, 26.44 mmol, 2.52 eq) were sequentially added thereto. The reaction mixture was stirred for 16 hours under nitrogen atmosphere. The reaction mixture was quenched by pouring into water (50 mL) and extracted with ethyl acetate (40 mL*3). The resulting organic phase was washed with saturated brine (40 mL) and dried over anhydrous sodium sulfate. The resulting mixture was filtered, and the filtrate was concentrated under reduced pressure to obtain a crude product, which was purified by column chromatography (petroleum ether:ethyl acetate=1:0 to 3:1) to obtain compound B-1-4. ¹H NMR (400 MHz, CDCl₃) δ ppm 7.67-7.72 (m, 4H), 7.37-7.46 (m, 6H), 7.00-7.03 (m, 1H), 6.98 (d, J=1.25 Hz, 1H), 5.41 (s, 2H), 4.84 (s, 2H), 3.42-3.47 (m, 2H), 1.06 (s, 9H), 0.85-0.90 (m, 2H), 0.03 (s, 9H). LCMS: m/z=467.2 [M+1]⁺.

### Step 4: Synthesis of Compound B-1-5

B-1-4 (10.9 g, 223.42 mmol, 1 eq) was dissolved in THE (120 mL), then cooled to 0° C., NBS (15 g, 84.28 mmol, 3.60 eq) was added thereto in batches, and then the reaction mixture was warmed to 20° C. and stirred for 16 hours. The reaction mixture was slowly poured into water (150 mL), stirred thoroughly, and extracted with ethyl acetate (100 mL*2). The organic phase was washed with saturated brine (200 mL), dried over anhydrous sodium sulfate, filtered, and the filtrate was concentrated under reduced pressure to obtain the crude product. The crude product was purified by neutral alumina column chromatography (petroleum ether:ethyl acetate=1:0 to 20:1) to obtain compound B-1-5. LCMS: m/z=622.9 [M+1]⁺.

### Step 5: Synthesis of Compound B-1-6

B-1-5 (2.4 g, 3.79 mmol, 1 eq) was dissolved in THE (24 mL) and cooled to −70° C. under nitrogen atmosphere. i-PrMgCl (2 M, 2.09 mL, 1.1 eq) was added dropwise thereto and stirred for 1.5 hours. Then DMF (55.47 g, 758.94 mmol, 58.39 mL, 200 eq) was added dropwise thereto, and the reaction mixture was warmed to 20° C. and stirred for 0.5 hours. The reaction mixture was slowly poured into a saturated ammonium chloride solution (200 mL) to quench, and extracted with ethyl acetate (200 mL*3). The organic phase was washed with saturated brine (200 mL), dried over anhydrous sodium sulfate, and filtered. The resulting filtrate was concentrated under reduced pressure to obtain the crude product. The crude product was purified by column chromatography (petroleum ether:ethyl acetate=1:0 to 10:1) to obtain compound B-1-6. ¹H NMR (400 MHz, CDCl₃) δ ppm 9.76 (s, 1H), 7.66 (dd, J=7.91, 1.38 Hz, 4H), 7.36-7.47 (m, 6H), 5.85 (s, 2H), 4.86 (s, 2H), 3.48-3.54 (m, 2H), 1.07 (s, 9H), 0.84-0.89 (m, 2H), 0.02-0.06 (m, 9H). LCMS: m/z=573.0 [M+1]⁺.

### Step 6: Synthesis of Compound B-1-7

B-1-6 (1.1 g, 1.97 mmol, 1 eq) was dissolved in dichloromethane (5 mL), and then TFA (8.98 g, 78.79 mmol, 5.83 mL, 40 eq) was added dropwise thereto and stirred at 20° C. for 16 hours. The reaction mixture was poured into a saturated sodium bicarbonate solution (70 mL) to adjust the pH of the reaction mixture to 5-6, and extracted with ethyl acetate (40 mL*3). The organic phase was washed with water (30 mL) and saturated brine (30 mL), dried over anhydrous sodium sulfate, and filtered. The resulting filtrate was concentrated to obtain a crude product, which was purified by column chromatography (petroleum ether:ethyl acetate=1:0 to 10:1) to obtain compound B-1-7. ¹H NMR (400 MHz, CDCl₃) δ ppm 9.65 (s, 1H), 7.63 (dd, J=7.94, 1.44 Hz, 4H), 7.40-7.50 (m, 6H), 4.83 (s, 2H), 1.13 (s, 9H). LCMS: m/z=442.9 [M+H]⁺.

### Step 7: Synthesis of Compound B-1-9

B-1-7 (0.02 g, 45.11 μmol, 1 eq) was dissolved in acetonitrile (0.5 mL), then cesium carbonate (16.17 mg, 49.62 μmol, 1.1 eq) and B-1-8 (11.24 mg, 67.66 μmol, 1.5 eq) were sequentially added thereto. The reaction mixture was heated to 80° C., stirred for 16 hours, and concentrated to obtain the crude product, which was purified by a preparative thin-layer chromatography silica gel plate (petroleum ether:ethyl acetate=5:1) to obtain B-1-9. ¹H NMR (400 MHz, CDCl₃) δ ppm 9.70 (s, 1H), 7.67 (ddd, J=12.38, 7.82, 1.31 Hz, 4H), 7.38-7.48 (m, 6H), 4.84-5.00 (m, 3H), 4.65-4.75 (m, 1H), 4.46-4.65 (m, 2H), 4.19 (dt, J=9.07, 6.10 Hz, 1H), 2.62-2.75 (m, 1H), 2.17-2.29 (m, 1H), 1.07-1.13 (m, 9H). LCMS: m/z=512.9[M+H]⁺.

### Step 8: Synthesis of Compound B-1-10

Sodium ethoxide (13 mg, 191.04 μmol, 3.77 eq) was dissolved in ethanol (0.5 mL), then ethyl thioglycolate (183.07 μmol, 20 μL, 3.62 eq) and B-1-9 (26 mg, 50.63 μmol, 1 eq) were sequentially added thereto, and the reaction mixture was heated to 80° C. and stirred for 20 hours. The pH of the reaction mixture was adjusted to 2-3 with 1 M hydrochloric acid, the aqueous phase was extracted with ethyl acetate (5 mL*3), and the organic phase was washed with saturated brine and then concentrated to obtain a crude product, which was purified by a preparative thin-layer chromatography silica gel plate (petroleum ether:ethyl acetate=5:1) to obtain B-1-10. ¹H NMR (400 MHz, CDCl₃) δ ppm 7.74 (s, 1H), 7.63-7.70 (m, 4H), 7.37-7.47 (m, 6H), 4.95 (s, 2H), 4.03-4.71 (m, 7H), 2.57-2.71 (m, 1H), 2.28-2.39 (m, 1H), 1.39 (t, J=7.15 Hz, 3H), 1.08 (s, 9H). LCMS: m/z=535.0 [M+H]⁺.

### Step 9: Synthesis of Compound B-1-11

B-1-10 (2 g, 3.15 mmol, purity of 84.3%, 1 eq) was added to THE (20 mL) in a dry reaction flask, then triethylamine trihydrofluoride (15.77 mmol, 2.57 mL, 5 eq) was added thereto, and the reaction mixture was stirred for 10 hours at 20° C. The reaction mixture was diluted with water (30 mL), and extracted with ethyl acetate (30 mL*3). After the phase separation, the organic phase was collected. The resulting organic phase was sequentially washed with saturated sodium bicarbonate (30 mL) and saturated brine (30 mL*3), dried over anhydrous sodium sulfate, filtered, and the resulting filtrate was concentrated under reduced pressure. Methyl tert-butyl ether (10 mL) and ethyl acetate (1 mL) were added thereto, and the resulting mixture was stirred and filtered. The filter cake was washed again with methyl tert-butyl ether (10 mL), and the resulting filter cake was concentrated under reduced pressure to obtain B-1-11. LCMS: m/z=297.1 [M+1]⁺.

### Step 10: Synthesis of Compound B-1

B-1-11 (0.8 g, 2.70 mmol, 1 eq), dichloromethane (10 mL), and triethylamine (819.51 mg, 8.10 mmol, 1.13 mL, 3 eq) were added to a dry reaction flask, the reaction system was replaced with nitrogen and cooled to 0° C., and methylsulfonyl chloride (463.86 mg, 4.05 mmol, 313.42 μL, 1.5 eq) was added thereto and stirred for 3 hours at 20° C. The reaction mixture was quenched by pouring into water (20 mL), and extracted with dichloromethane (20 mL*3). The organic phase was collected after phase separation, sequentially washed with saturated brine (20 mL), dried over anhydrous sodium sulfate, and filtered. The resulting filtrate was concentrated under reduced pressure to obtain the crude product. The crude product was purified by column chromatography (petroleum ether:ethyl acetate=1:0 to 3:1) to obtain B-1. LCMS: m/z=315.1 [M+1]⁺.

### Reference Example 2: Fragment B-2

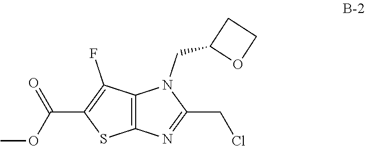 [SMILES: COC(=O)c1sc2nc(CCl)n(C[C@@H]3CCO3)c2c1F]

### Synthetic Route:

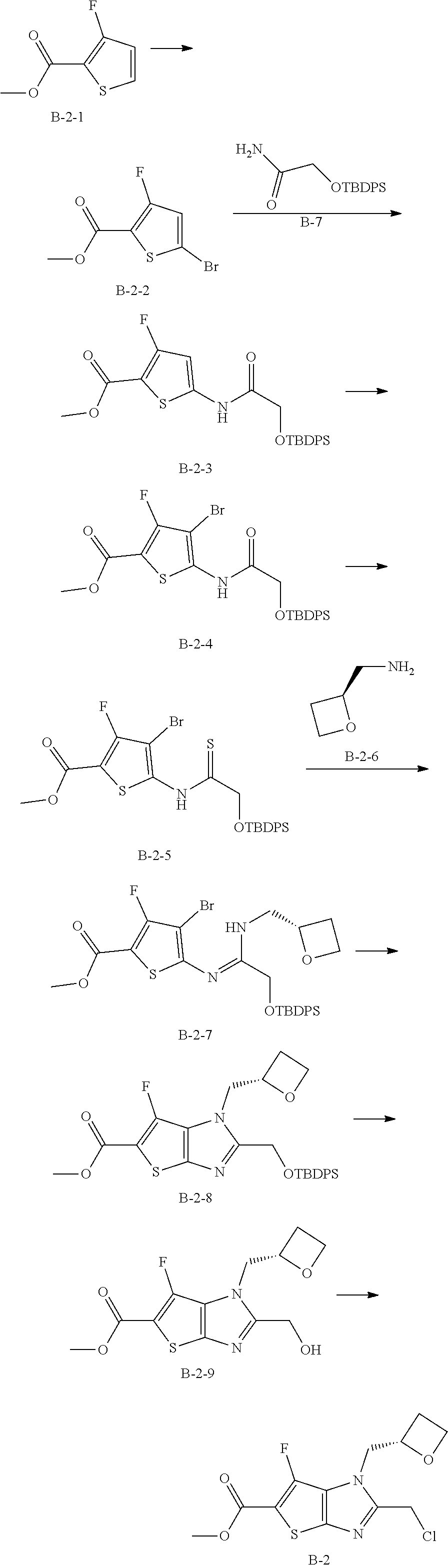

### Step 1: Synthesis of Compound B-2-2

B-2-1 (2.00 g, 12.49 mmol, 1 eq) and anhydrous tetrahydrofuran (100 mL) were added to a reaction flask, and 2,2,6,6-tetramethylpiperidinylmagnesium chloride lithium chloride complex (1 M, 24.00 mL, 1.92 eq) was added thereto at −40° C. The reaction system was stirred for 0.5 hours at −40° C., carbon tetrabromide (4.14 g, 12.49 mmol, 1 eq) was added thereto, and the reaction system was stirred for 0.5 hours at −40° C. and then gradually warmed to 20° C. and stirred for 11 hours. The reaction mixture was quenched with hydrochloric acid (0.5 M, 10 mL) and extracted with ethyl acetate (50 mL*3). The organic phases were combined, and the resulting organic phase was washed with saturated brine (50 mL), dried over anhydrous sodium sulfate, and filtered. The filtrate was concentrated to obtain the crude product. The crude product was purified by column chromatography (petroleum ether:ethyl acetate=1:0 to 20:1) to obtain compound B-2-2. LCMS: m/z=238.9 [M+1]⁺.

### Step 2: Synthesis of Compound B-2-3

B-2-2 (1.70 g, 7.11 mmol, 1 eq), B-7 (2.23 g, 7.11 mmol, 1.0 eq), and potassium carbonate (1.97 g, 14.22 mmol, 2 eq) were dissolved in anhydrous toluene (50 mL), then tris(dibenzylideneacetone)dipalladium (0.65 g, 709.82 μmol, 0.10 eq) and Xantphos (0.82 g, 1.42 mmol, 0.2 eq) were added thereto under nitrogen atmosphere, and then the reaction mixture was heated to 100° C. and stirred for 12 hours. The reaction mixture was filtered, and the filtrate was added with water (50 mL) and extracted with ethyl acetate (50 mL*3). The organic phases were combined, and the resulting organic phase was washed with saturated brine (50 mL), dried over anhydrous sodium sulfate, filtered, and the filtrate was concentrated to obtain the crude product. The crude product was purified by column chromatography (petroleum ether:ethyl acetate=1:0 to 4:1) to obtain B-2-3. LCMS: m/z=472.1 [M+1]⁺.

### Step 3: Synthesis of Compound B-2-4

B-2-3 (2.60 g, 2.84 mmol, purity of 51.53%, 1 eq) and NBS (1.00 g, 5.62 mmol, 1.98 eq) were dissolved in anhydrous THF (50 mL), and the reaction system was stirred for 12 hours at 20° C. The reaction mixture was added with saturated sodium bicarbonate solution (50 mL) and extracted with ethyl acetate (50 mL*3). The organic phases were combined, and the resulting organic phase was washed with saturated brine (50 mL), dried over anhydrous sodium sulfate, and filtered, the filtrate was concentrated to obtain the crude product. The crude product was purified by column chromatography (petroleum ether:ethyl acetate=1:0 to 10:1) to obtain B-2-4. ¹H NMR (400 MHz, CDCl₃) δ ppm 9.65 (br s, 1H), 7.66 (br d, J=6.78 Hz, 4H), 7.42-7.50 (m, 6H), 4.31 (s, 2H), 3.89 (s, 3H), 1.19 (s, 9H). LCMS: m/z=550.0 [M+1]⁺.

### Step 4: Synthesis of Compound B-2-5

B-2-4 (1.80 g, 3.27 mmol, 1 eq) and Lawesson's reagent (1.35 g, 3.34 mmol, 1.02 eq) were dissolved in anhydrous dioxane (30 mL), and the reaction system was stirred for 6 hours at 110° C. The reaction mixture was cooled to room temperature and concentrated to obtain the crude product. The crude product was purified by column chromatography (petroleum ether: ethyl acetate=1:0 to 10:1) to obtain compound B-2-5. ¹H NMR (400 MHz, CDCl₃) δ ppm 11.18 (br s, 1H), 7.66 (dd, J=7.91, 1.38 Hz, 4H), 7.40-7.51 (m, 6H), 4.60 (s, 2H), 3.91 (s, 3H), 1.20 (s, 9H). LCMS: m/z=566.0 [M+1]⁺.

### Step 5: Synthesis of Compound B-2-7

B-2-5 (1.50 g, 2.65 mmol, 1 eq), B-2-6 (0.60 g, 6.89 mmol, 2.60 eq), and silver acetate (0.90 g, 5.39 mmol, 276.07 μL, 2.04 eq) were dissolved in anhydrous DMF (20 mL), and the reaction system was stirred for 16 hours at 20° C. The reaction mixture was filtered, and the filtrate was added with water (50 mL) and extracted with ethyl acetate (50 mL*3). The organic phases were combined, washed with saturated brine (50 mL), dried over anhydrous sodium sulfate, filtered, and the resulting filtrate was concentrated to obtain the crude product. The crude product was purified by column chromatography (petroleum ether:ethyl acetate=1:0 to 5:1) to obtain B-2-7. ¹H NMR (400 MHz, CDCl₃) δ ppm 7.63 (br t, J=5.65 Hz, 4H), 7.39-7.52 (m, 6H), 6.95 (br s, 1H), 5.05-5.17 (m, 1H), 4.68-4.78 (m, 1H), 4.53 (dt, J=9.22, 5.93 Hz, 1H), 4.31-4.45 (m, 2H), 3.77-3.88 (m, 4H), 3.56-3.66 (m, 1H), 2.67-2.78 (m, 1H), 2.55-2.66 (m, 1H), 1.10 (s, 9H). LCMS: m/z=619.1 [M+1]⁺.

### Step 6: Synthesis of Compound B-2-8

B-2-7 (0.80 g, 1.29 mmol, 1 eq) and N,N′-dimethylethylenediamine (0.16 g, 1.82 mmol, 195.36 μL, 1.41 eq) were dissolved in acetonitrile (10 mL), then cuprous iodide (0.16 g, 840.12 μmol, 0.65 eq) was added thereto under nitrogen atmosphere, and the reaction system was stirred at 80° C. for 10 hours. The reaction mixture was filtered, added with water (20 mL), and extracted with ethyl acetate (20 mL*3). The organic phases were combined, washed with saturated brine (20 mL), dried over anhydrous sodium sulfate, filtered, and the filtrate was concentrated to obtain the crude product. The crude product was purified by a preparative thin-layer chromatography silica gel plate (petroleum ether:ethyl acetate=3:1) to obtain compound B-2-8. LCMS: m/z=539.2 [M+1]⁺.

### Step 7: Synthesis of Compound B-2-9

B-2-8 (0.40 g, 742.52 μmol, 1 eq) was dissolved in anhydrous tetrahydrofuran (5 mL), then tetrabutylammonium fluoride (1 M, 1.00 mL, 1.35 eq) was added thereto, and the reaction system was stirred for 1 hour at 20° C. The reaction mixture was added with water (20 mL) and extracted with ethyl acetate (20 mL*3). The organic phases were combined, washed with saturated brine (20 mL), dried over anhydrous sodium sulfate, filtered, and the filtrate was concentrated to obtain the crude product. The crude product was purified by column chromatography (petroleum ether:ethyl acetate=1:0 to 0:1) to obtain compound B-2-9. 1H NMR (400 MHz, CDCl₃) δ ppm 5.19 (qd, J=6.90, 2.89 Hz, 1H), 4.80-4.90 (m, 2H), 4.67-4.74 (m, 1H), 4.40-4.56 (m, 3H), 3.91 (s, 3H), 2.78-2.89 (m, 1H), 2.48-2.58 (m, 1H). LCMS: m/z=301.1 [M+1]⁺.

### Step 8: Synthesis of Compound B-2

B-2-9 (30 mg, 99.90 μmol, 1 eq) was dissolved in anhydrous dichloromethane (2 mL), then methanesulfonyl chloride (261.89 μmol, 20.27 μL, 2.62 eq) and triethylamine (296.47 mol, 41.27 μL, 2.97 eq) were added thereto at 0° C. (ice-water bath), and the reaction system was stirred for 1 hour at 20° C. The reaction mixture was quenched with saturated sodium bicarbonate aqueous solution (0.5 mL) and concentrated to obtain compound B-2. LCMS: m/z=318.8 [M+1]⁺.

### Reference Example 3: Fragments B-3 and B-4

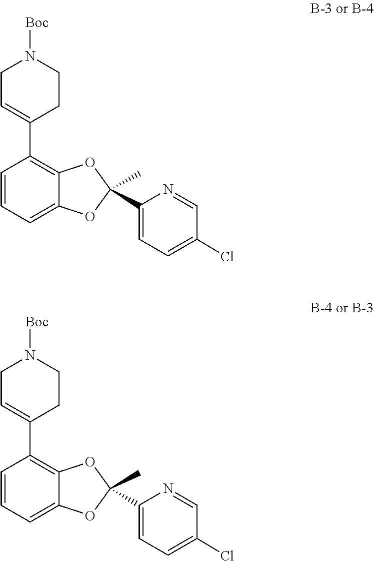 [SMILES: CC(C)(C)OC(=O)N1CC=C(c2cccc3c2O[C@@](C)(c2ccc(Cl)cn2)O3)CC1 ; CC(C)(C)OC(=O)N1CC=C(c2cccc3c2O[C@](C)(c2ccc(Cl)cn2)O3)CC1]

### Synthetic Route:

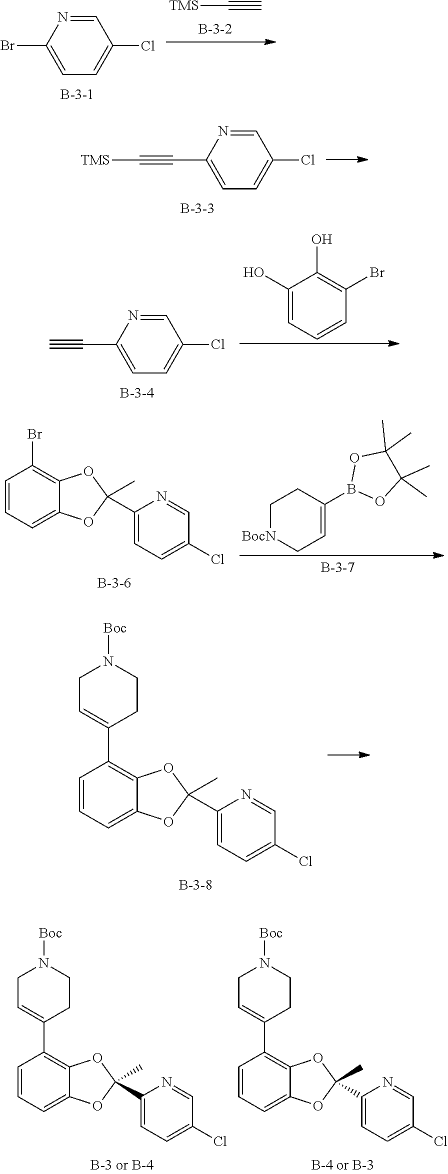

### Step 1: Synthesis of Compound B-3-3

B-3-2 (519.64 mmol, 71.99 mL, 2 eq), cuprous iodide (989.65 mg, 5.20 mmol, 0.02 eq), bis(triphenylphosphine)palladium(II) chloride (1.82 g, 2.60 mmol, 0.01 eq), and B-3-1 (50 g, 259.82 mmol, 1 eq) were dissolved in triethylamine (550 mL), and the reaction mixture was reacted for 8 hours at 60° C. under nitrogen atmosphere. The reaction mixture was diluted with saturated brine (400 mL) and extracted with ethyl acetate (400 mL*3), and the organic phase was collected after phase separation, dried over anhydrous sodium sulfate, filtered, and concentrated under reduced pressure to obtain a crude product. The crude product was purified by column chromatography (petroleum ether:ethyl acetate=1:0 to 20:1) to obtain B-3-3. LCMS: m/z=210.2 [M+1]⁺.

### Step 2: Synthesis of Compound B-3-4

Potassium hydroxide (6.29 g, 95.35 mmol, purity of 85%, 1 eq) was added to a solution of B-3-3 (20 g, 95.35 mmol, 1 eq) in methanol (400 mL), and the reaction mixture was stirred for 1 hour at 25° C. The reaction mixture was poured into saturated brine (500 mL) and extracted with ethyl acetate (600 mL*3). The organic phases were combined. The organic phase was dried over anhydrous sodium sulfate, filtered, and the resulting filtrate was concentrated to obtain B-3-4. LCMS: m/z=138.2 [M+1]⁺.

### Step 3: Synthesis of Compound B-3-6

Triruthenium dodecycarbonyl (464.74 mg, 726.92 μmol, 0.02 eq), B-3-5 (6.87 g, 36.35 mmol, 1 eq), and B-3-4 (5 g, 36.35 mmol, 1 eq) were dissolved in toluene (100 mL), and the reaction mixture was replaced three times with nitrogen and reacted for 16 hours at 100° C. The reaction mixture was filtered with diatomite, and the filter cake was washed with ethyl acetate (20 mL*3). The filtrates were combined and concentrated under reduced pressure to obtain the crude product. The crude product was purified by column chromatography (petroleum ether:ethyl acetate=1:0 to 50:1) to obtain B-3-6. LCMS: m/z=326.0 [M+1]⁺.

### Step 4: Synthesis of Compound B-3-8

B-3-6 (4.5 g, 13.78 mmol, 1 eq), B-3-7 (6.39 g, 20.67 mmol, 1.5 eq), [1,1′-bis(diphenylphosphino)ferrocene]dichloropalladium(II) (2.02 g, 2.76 mmol, 0.2 eq), and potassium carbonate (7.62 g, 55.12 mmol, 4 eq) were dissolved in dioxane (100 mL)/water (10 mL), and the reaction mixture was replaced three times with nitrogen and reacted for 12 hours at 90° C. The reaction mixture was poured into water (200 mL) and extracted with ethyl acetate (300 mL*3). The organic phases were combined, dried over anhydrous sodium sulfate, filtered, and the resulting filtrate was concentrated to obtain the crude product. The crude product was purified by column chromatography (petroleum ether:ethyl acetate=1:0 to 10:1) to obtain B-3-8. LCMS: m/z=373.1 [M−55]⁺.

### Step 5: Synthesis of Compounds B-3 and B-4

B-3-8 was purified by preparative SFC [column model: DAICEL CHIRALPAK IC (250 mm*30 mm*10 m); mobile phase: phase A was supercritical carbon dioxide, phase B was ethanol (0.1% ammonia water); gradient (B %): 35% to 35%] to obtain compound B-3 and compound B-4.

According to SFC detection [column model: Chiralpak AD-3, 50×4.6 mm I.D., 3 m); mobile phase: phase A was supercritical carbon dioxide, phase B was ethanol (0.1% isopropylamine); gradient (B %): 5% to 50%], the retention time of the compound B-3 was 0.920 minutes, and the e.e. value was 100%; and the retention time of the compound B-4 was 1.197 minutes, and the e.e. value was 98.4%.

### Reference Example 4: Fragments B-5 and B-6

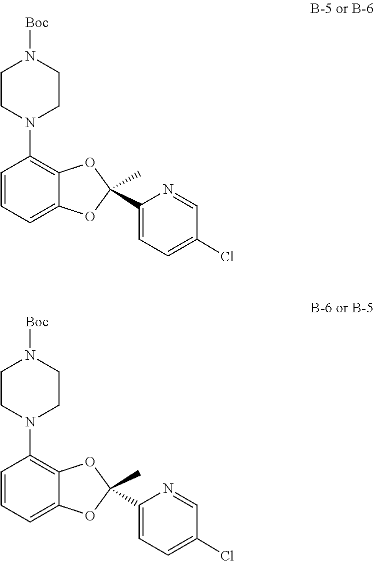 [SMILES: CC(C)(C)OC(=O)N1CCN(c2cccc3c2O[C@@](C)(c2ccc(Cl)cn2)O3)CC1 ; CC(C)(C)OC(=O)N1CCN(c2cccc3c2O[C@](C)(c2ccc(Cl)cn2)O3)CC1]

### Synthetic Route:

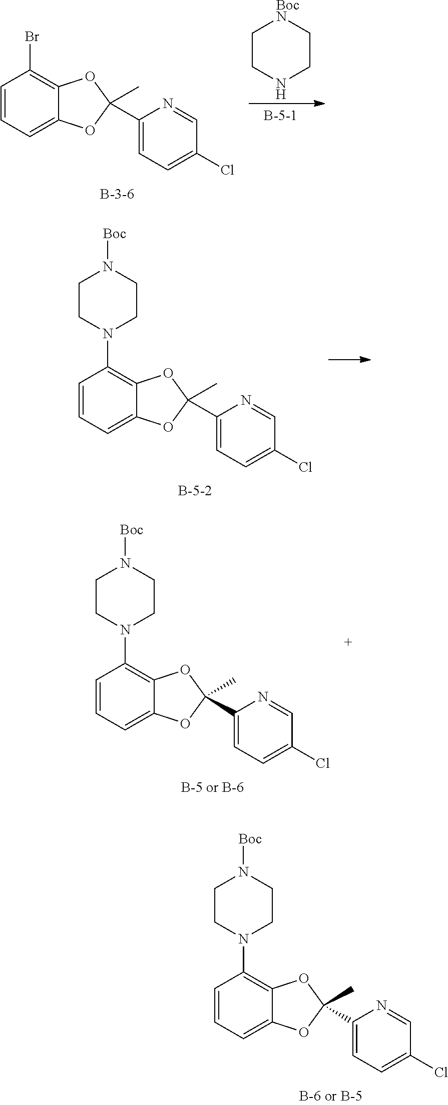 [SMILES: CC1(c2ccc(Cl)cn2)Oc2cccc(Br)c2O1 ; CC(C)(C)OC(=O)N1CCNCC1 ; CC(C)(C)OC(=O)N1CCN(c2cccc3c2OC(C)(c2ccc(Cl)cn2)O3)CC1 ; CC(C)(C)OC(=O)N1CCN(c2cccc3c2O[C@@](C)(c2ccc(Cl)cn2)O3)CC1 ; CC(C)(C)OC(=O)N1CCN(c2cccc3c2O[C@](C)(c2ccc(Cl)cn2)O3)CC1]

### Step 1: Synthesis of Compound B-5-2

B-3-6 (1.6 g, 4.90 mmol, 1 eq), B-5-1 (1.31 g, 5.88 mmol, 1.2 eq), and cesium carbonate (4.79 g, 14.70 mmol, 3 eq) were added to toluene (16 mL), the system was replaced with nitrogen, and then palladium acetate (55.00 mg, 244.97 μmol, 0.05 eq) and BINAP (213.55 mg, 342.96 μmol, 0.07 eq) were added thereto. The reaction mixture was heated to 100° C., and stirred for 10 hours. The reaction mixture was diluted with water (20 mL) and extracted with ethyl acetate (20 mL*3). The organic phase was collected after phase separation, washed with saturated brine (20 mL*3), and dried over anhydrous sodium sulfate, and the resulting filtrate was concentrated under reduced pressure to obtain the crude product. The crude product was purified by column chromatography (petroleum ether:ethyl acetate=1:0 to 5:1) to obtain B-5-2. LCMS: m/z=432.2 [M+1]⁺.

Step 2: Synthesis of compounds B-5 and B-6

B-5-2 was purified by preparative SFC [column model: DAICEL CHIRALPAK IC (250 mm*30 mm*10 m); mobile phase: phase A was supercritical carbon dioxide, and phase B was methanol (neutral); gradient (B %): 35% to 35%] to obtain compound B-5 and compound B-6.

According to SFC detection [column model: Chiralpak IG-3, 50×4.6 mm I.D., 3 m); mobile phase: phase A was supercritical carbon dioxide, phase B was methanol (0.1% isopropylamine); gradient (B %): 5% to 50%], the retention time of the compound B-5 was 1.015 minutes, and the e.e. value was 99.5%; and the retention time of the compound B-6 was 1.134 minutes, and the e.e. value was 99.6%.

### Reference Example 5: Fragment B-7

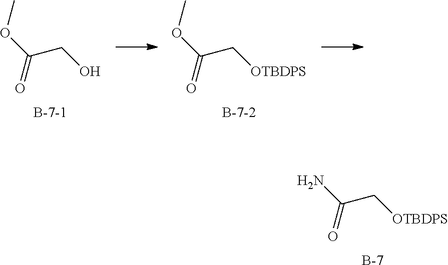

### Step 1: Synthesis of Compound B-7-2

B-7-1 (10.00 g, 111.02 mmol, 1 eq), tert-butyldiphenylchlorosilane (36.62 g, 133.22 mmol, 34.22 mL, 1.2 eq), and imidazole (8.92 g, 131.00 mmol, 1.18 eq) were dissolved in anhydrous DMF (150.00 mL) and the reaction system was stirred for 3 hours at 0° C. The reaction mixture was concentrated to obtain the crude product, which was dissolved with ethyl acetate (200 mL), sequentially washed with water (200 mL*2) and saturated brine (30 mL). The organic phase was dried over anhydrous sodium sulfate, filtered, and the filtrate was concentrated to obtain the crude product. The crude product was purified by silica gel column chromatography (petroleum ether:ethyl acetate=1:0 to 10:1) to obtain compound B-7-2. ¹H NMR (400 MHz, CDCl₃) δ ppm 7.69 (dd, J=7.88, 1.38 Hz, 4H), 7.37-7.45 (m, 6H), 4.26 (s, 2H), 3.69 (s, 3H), 1.10 (s, 9H).

### Step 2: Synthesis of Compound B-7

In a dry reaction flask, B-7-2 (220 g, 669.76 mmol, 1 eq) was added to a methanol solution of ammonia (7 M, 1.58 L, 16.5 eq) and stirred for 10 hours at 50° C. The reaction mixture was concentrated and purified by silica gel column chromatography (petroleum ether:ethyl acetate=1:0 to 3:1) to obtain compound B-7. ¹H NMR (400 MHz, DMSO-d₆) δ ppm 7.64 (dd, J=7.91, 1.63 Hz, 4H), 7.42-7.52 (m, 6H), 7.40 (br s, 1H), 7.11 (br s, 1H), 3.94 (s, 2H), 1.02 (s, 9H).

### Reference Example 6: Fragment B-8

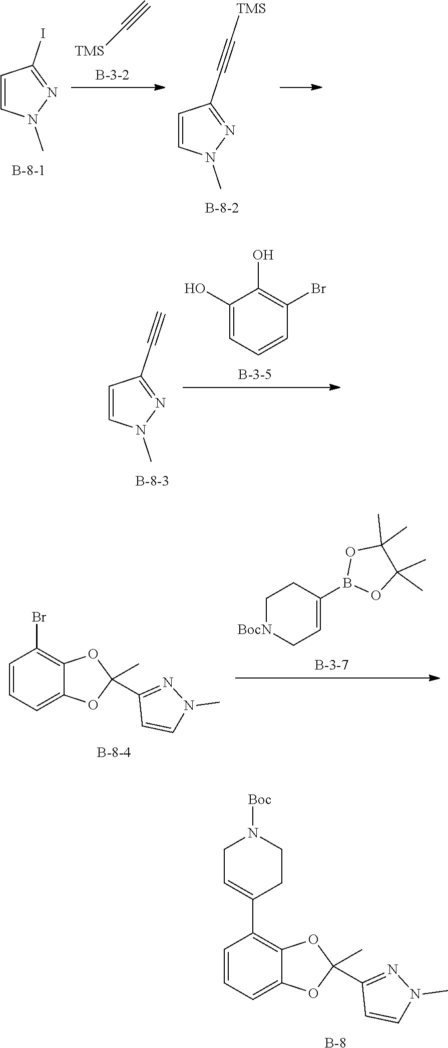

### Step 1: Synthesis of B-8-2

B-8-1 (8.8 g, 42.31 mmol, 1 eq) was dissolved in acetonitrile (88.0 mL), and then triethylamine (35.3 mL), cuprous iodide (161.15 mg, 0.85 mmol, 0.02 eq), bis(triphenylphosphine)palladium(II) chloride (0.3 g, 0.42 mmol, 0.01 eq), and B-3-2 (59.23 mmol, 8.21 mL, 1.4 eq) were sequentially added thereto, and the reaction mixture was reacted for 3 hours at 75° C. under nitrogen atmosphere. The reaction mixture was diluted with saturated brine (80 mL) and extracted with ethyl acetate (80 mL*3), and the organic phase was collected after phase separation, dried over anhydrous sodium sulfate, and concentrated under reduced pressure. The crude product was purified by column chromatography (petroleum ether:ethyl acetate=1:0 to 10:1) to obtain B-8-2. LCMS: m/z=179.0 [M+1]⁺.

### Step 2: Synthesis of Compound B-8-3

B-8-2 (6.5 g, 36.45 mmol, 1 eq) was dissolved in tetrahydrofuran (60.0 mL), then a solution of potassium hydroxide (2.05 g) in water (10.0 mL) was added thereto, and stirred at 25° C. for 12 hours. The reaction mixture was diluted with saturated brine (70 mL) and extracted with ethyl acetate (70 mL*2). The organic phases were combined, washed with water (70 mL), dried over anhydrous sodium sulfate, and then filtered, and the filtrate was concentrated to obtain B-8-3. ¹H NMR (400 MHz, CDCl₃) δ ppm 7.31 (d, J=2.26 Hz, 1H), 6.42 (d, J=2.26 Hz, 1H), 3.91 (s, 3H), 3.06 (s, 1H).

### Step 3: Synthesis of Compound B-8-4

B-8-3 (2.6 g, 24.50 mmol, 1 eq) and B-3-5 (4.17 g, 22.05 mmol, 0.9 eq) were dissolved in toluene (30.0 mL), then triruthenium dodecycarbonyl (313.26 mg, 0.49 mmol, 0.02 eq) was added thereto under nitrogen atmosphere, and the reaction mixture was reacted for 12 hours at 100° C. The reaction mixture was filtered with diatomite, and the filter cake was washed with ethyl acetate (100 mL*2). The filtrates were combined and concentrated under reduced pressure to obtain the crude product. The crude product was purified by column chromatography (petroleum ether:ethyl acetate=1:0 to 4:1) to obtain B-8-4. LCMS: m/z=295.0 [M+1]⁺.

### Step 4: Synthesis of Compound B-8

B-8-4 (7.2 g, 24.40 mmol, 1 eq), B-3-7 (9.05 g, 29.28 mmol, 1.2 eq), and sodium carbonate (10.34 g, 97.58 mmol, 4 eq) were dissolved in dioxane (70 mL)/water (30 mL), then tetrakis(triphenylphosphine)palladium (1.41 g, 1.22 mmol, 0.05 eq) was added thereto under nitrogen atmosphere, and the reaction mixture was reacted for 2 hours at 100° C. The reaction mixture was filtered with diatomite, and the filter cake was washed with ethyl acetate (70 mL*3). The resulting filtrate was washed with water (70 mL), and then concentrated under reduced pressure to obtain the crude product. The crude product was purified by column chromatography (petroleum ether:ethyl acetate=1:0 to 4:1) to obtain B-8. LCMS: m/z=420.0 [M+23]⁺.

### Reference Example 7: Fragment B-9

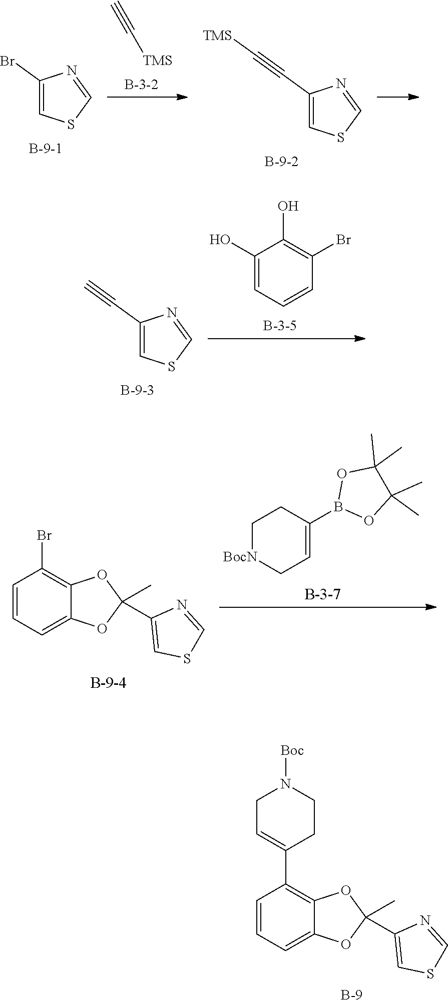

### Step 1: Synthesis of B-9-2

B-9-1 (6.94 g, 42.31 mmol, 1 eq) was dissolved in acetonitrile (88.0 mL), and then triethylamine (35.3 mL), cuprous iodide (161.15 mg, 0.85 mmol, 0.02 eq), bis(triphenylphosphine)palladium(II) chloride (0.3 g, 0.42 mmol, 0.01 eq), and B-3-2 (59.23 mmol, 8.21 mL, 1.4 eq) were sequentially added thereto, and the reaction mixture was reacted for 3 hours at 75° C. under nitrogen atmosphere. The reaction mixture was diluted with saturated brine (80 mL) and extracted with ethyl acetate (80 mL*3), and the organic phase was collected after phase separation, dried over anhydrous sodium sulfate, and concentrated under reduced pressure. The crude product was purified by column chromatography (petroleum ether:ethyl acetate=1:0 to 15:1) to obtain B-9-2. ¹H NMR (400 MHz, DMSO-d₆) δ ppm 9.06 (d, J=2.0 Hz, 1H), 8.05 (s, 1H), 0.20 (s, 9H). LCMS: m/z=181.8 [M+1]⁺.

### Step 2: Synthesis of Compound B-9-3

B-9-2 (6.61 g, 36.45 mmol, 1 eq) was dissolved in tetrahydrofuran (60.0 mL), then a solution of potassium hydroxide (2.05 g) in water (10.0 mL) was added thereto, and stirred at 25° C. for 12 hours. The reaction mixture was diluted with saturated brine (70 mL) and extracted with ethyl acetate (70 mL*2). The organic phases were combined, washed with water (70 mL), dried over anhydrous sodium sulfate, and then filtered, and the filtrate was concentrated to obtain B-9-3. ¹H NMR (400 MHz, CDCl₃) δ ppm 8.76 (s, 1H), 7.58 (s, 1H), 3.15 (s, 1H).

### Step 3: Synthesis of Compound B-9-4

B-9-3 (2.67 g, 24.50 mmol, 1 eq) and B-3-5 (4.17 g, 22.05 mmol, 0.9 eq) were dissolved in toluene (30.0 mL), then triruthenium dodecycarbonyl (313.26 mg, 0.49 mmol, 0.02 eq) was added thereto under nitrogen atmosphere, and the reaction mixture was reacted for 12 hours at 100° C. The reaction mixture was filtered with diatomite, and the filter cake was washed with ethyl acetate (100 mL*2). The filtrates were combined and concentrated under reduced pressure to obtain the crude product. The crude product was purified by column chromatography (petroleum ether:ethyl acetate=1:0 to 6:1) to obtain B-9-4. LCMS: m/z=298.1 [M+1]⁺.

### Step 4: Synthesis of Compound B-9-5

B-9-4 (7.3 g, 24.40 mmol, 1 eq), B-3-7 (9.05 g, 29.28 mmol, 1.2 eq), and sodium carbonate (10.34 g, 97.58 mmol, 4 eq) were dissolved in dioxane (70 mL)/water (30 mL), then tetrakis(triphenylphosphine)palladium (1.41 g, 1.22 mmol, 0.05 eq) was added thereto under nitrogen atmosphere, and the reaction mixture was reacted for 2 hours at 100° C. The reaction mixture was filtered with diatomite, and the filter cake was washed with ethyl acetate (70 mL*3). The resulting filtrate was washed with water (70 mL), and then concentrated under reduced pressure to obtain the crude product. The crude product was purified by column chromatography (petroleum ether:ethyl acetate=1:0 to 6:1) to obtain B-9-5. LCMS: m/z=401.2 [M+1]⁺.

### Reference Example 8: Fragment B-10

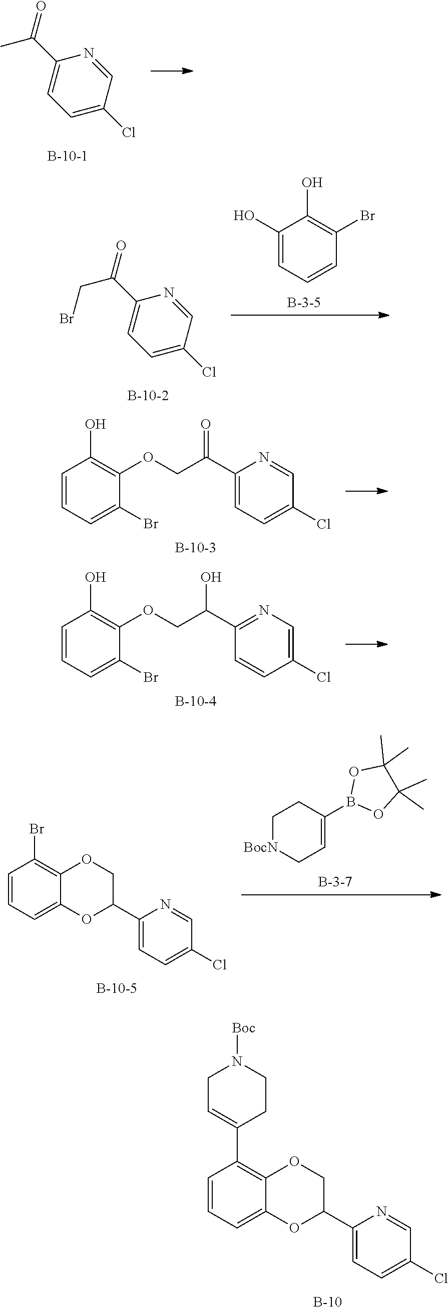

### Step 1: Synthesis of B-10-2

B-10-1 (20 g, 128.55 mmol, 1 eq) was dissolved in THE (200 mL), and the reaction system was replaced with nitrogen. Phenyltrimethylammonium tribromide (50.74 g, 134.98 mmol, 1.05 eq) was then added to the reaction flask. The reaction mixture was gradually heated to 50° C. and stirred for 12 hours. The reaction mixture was quenched with water (50 mL) and then extracted by adding ethyl acetate (50 mL*2) and the organic phase was collected. The organic phase was washed with saturated brine (50 mL), dried over anhydrous sodium sulfate, filtered, and then concentrated under reduced pressure. A mixture (150 mL) of petroleum ether and ethyl acetate with a ratio of 10:1 was added to the crude product, and the reaction mixture was stirred for 1 hour at 25° C. The reaction mixture was filtered. The filter cake was collected and dried to obtain compound B-10-2, which was directly used in the next reaction step. LCMS: m/z=234.0 [M+1]⁺.

### Step 2: Synthesis of B-10-3

B-10-2 (13.70 g, 72.50 mmol, 1 eq) was dissolved in anhydrous DMF (170 mL), then potassium carbonate (10.02 g, 72.50 mmol, 1 eq) was added thereto, and the reaction mixture was stirred for 18 hours at 25° C. The reaction mixture was diluted with ethyl acetate (170 mL), then sequentially washed with water (170 mL) and saturated aqueous sodium chloride solution (170 mL). The organic phase was dried over anhydrous sodium sulfate and filtered, and the filtrate was concentrated under reduced pressure. The crude product was purified by column chromatography (petroleum ether:ethyl acetate=50:1 to 10:1) to obtain B-10-3. ¹H NMR (400 MHz, DMSO-d₆) δ ppm 8.69 (d, J=2.4 Hz, 1H), 8.06 (dd, J=2.4, 8.5 Hz, 1H), 7.90 (s, 1H), 7.79 (d, J=8.5 Hz, 1H), 7.18 (d, J=7.9 Hz, 1H), 7.01 (d, J=8.1 Hz, 1H), 6.90-6.78 (m, 1H), 4.40 (s, 2H). LCMS: m/z=342.0 [M+1]⁺.

### Step 3: Synthesis of B-10-4

B-10-3 (13 g, 37.95 mmol, 1 eq) was dissolved in EtOH (260 mL), the reaction system was replaced with nitrogen, and NaBH₄ (2.87 g, 75.90 mmol, 2 eq) was added to the reaction flask, and the reaction mixture was stirred for 2 hours at 25° C. The reaction mixture was quenched with saturated aqueous ammonium chloride solution (300 mL) and extracted with ethyl acetate (300 mL*2). The organic phase was collected, washed with saturated aqueous sodium chloride solution (300 mL), dried over anhydrous sodium sulfate, and filtered. The filtrate was concentrated under reduced pressure to obtain the crude product. The crude product was purified by column chromatography (petroleum ether:ethyl acetate=100:1 to 10:1) to obtain B-10-4. ¹H NMR (400 MHz, DMSO-d₆) δ ppm 10.31-9.59 (m, 1H), 8.57 (d, J=2.3 Hz, 1H), 7.97 (dd, J=2.5, 8.4 Hz, 1H), 7.64 (d, J=8.5 Hz, 1H), 7.00-6.95 (m, 1H), 6.90-6.85 (m, 2H), 4.97 (dd, J=4.3, 6.4 Hz, 1H), 4.24 (dd, J=4.3, 10.1 Hz, 1H), 4.12 (dd, J=6.6, 10.0 Hz, 1H). LCMS: m/z=344.0 [M+1]⁺.

### Step 4: Synthesis of B-10-5

Compound B-10-4 (3 g, 8.71 mmol, 1 eq), triphenylphosphine (4.57 g, 17.41 mmol, 2 eq), and tetrahydrofuran (60 mL) were added to a dry reaction flask, and the system was replaced with nitrogen. The mixture was cooled to 0° C., then a solution of DIAD (3.52 g, 17.41 mmol, 2 eq) in tetrahydrofuran (15 mL) was added thereto, and the reaction mixture was stirred for 12 hours at 20° C. The reaction system was quenched with water (100 mL), then added with sodium hypochlorite solution (10 mL), and stirred for 10 minutes. The reaction mixture was extracted with ethyl acetate (100 mL×2), and the organic phases were combined after phase separation. The organic phase was washed with saturated brine (100 mL), dried over anhydrous sodium sulfate, filtered, and concentrated under reduced pressure to obtain the crude product. The crude product was purified by column chromatography (petroleum ether:ethyl acetate=100:1 to 10:1) to obtain B-10-5. LCMS: m/z=326.0 [M+1]⁺.

### Step 5: Synthesis of B-10

Compound B-10-5 (1 g, 3.06 mmol, 1 eq), B-3-7 (2.84 g, 9.19 mmol, 3 eq), 1,4-dioxane (25 mL), H₂O (5 mL), sodium carbonate (973.66 mg, 9.19 mmol, 3 eq), and Pd(PPh₃)₄ (353.84 mg, 306.21 μmol, 0.1 eq) were sequentially added to a dry reaction flask, and the reaction system was replaced with nitrogen and stirred for 10 hours at 90° C. The reaction system was quenched with water (50 mL), then sodium hypochlorite aqueous solution (10 mL) was added thereto and stirred for 10 minutes, then the resulting mixture was extracted with ethyl acetate (50 mL×2), and the organic phases were combined after phase separation. The organic phase was washed with saturated brine (50 mL), dried over anhydrous sodium sulfate, filtered, and concentrated under reduced pressure to obtain the crude product. The crude product was purified by column chromatography (petroleum ether:ethyl acetate=100:1 to 10:1) to obtain B-10. LCMS: m/z=451.2 [M+23]⁺.

### Reference Example 9: Fragment B-11

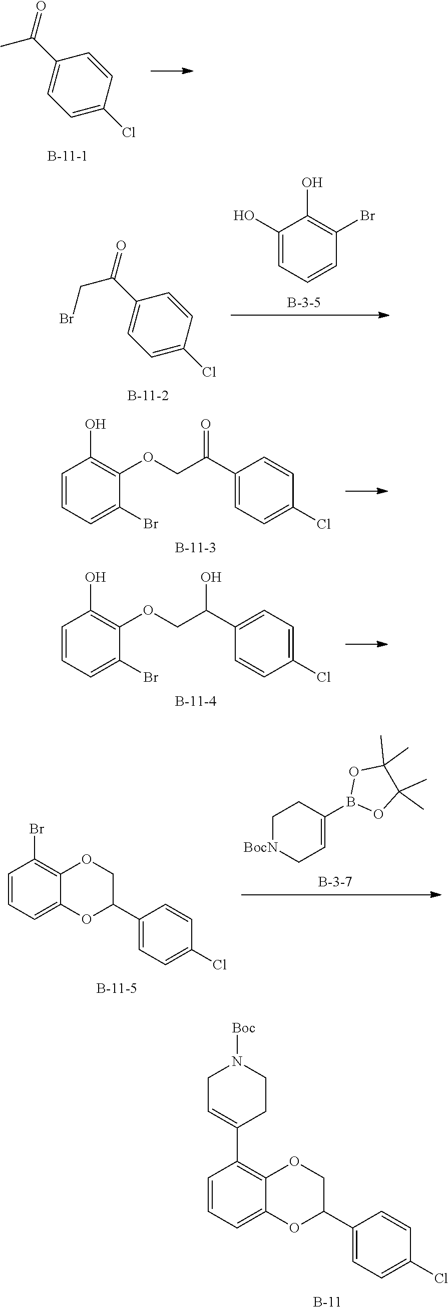

### Step 1: Synthesis of B-11-2

Compound B-11-1 (29 g, 187.59 mmol, 1 eq) was dissolved in tetrahydrofuran (290 mL), and the reaction system was replaced with nitrogen. Phenyltrimethylammonium tribromide (74.05 g, 196.97 mmol, 1.05 eq) was then added to the reaction flask. The reaction mixture was gradually heated to 50° C. and stirred for 12 hours. The reaction mixture was quenched with water (500 mL) and then extracted by adding ethyl acetate (500 mL*2) and the organic phase was collected. The organic phase was washed with saturated brine (500 mL), dried over anhydrous sodium sulfate, filtered, and concentrated under reduced pressure. A mixture (150 mL) of petroleum ether and ethyl acetate with a ratio of 10:1 was added to the crude product, and the reaction mixture was stirred for 1 hour at 25° C. The resulting mixture was filtered. The filter cake was collected and dried to obtain B-11-2, which was directly used in the next reaction step. LCMS: m/z=233.0 [M+H]⁺.

### Step 2: Synthesis of B-11-3

B-11-2 (10 g, 42.83 mmol, 1 eq) and B-3-5 (8.09 g, 42.83 mmol, 1 eq) were dissolved in anhydrous DMF (100 mL), then potassium carbonate (5.92 g, 42.83 mmol, 1 eq) was added thereto, and the reaction mixture was stirred for 18 hours at 25° C. The reaction mixture was diluted with ethyl acetate (10 mL), then sequentially washed with water (10 mL) and saturated aqueous sodium chloride solution (10 mL). The organic phase was dried over anhydrous sodium sulfate and filtered, and the filtrate was concentrated under reduced pressure. The crude product was purified by column chromatography (petroleum ether:ethyl acetate=1:0 to 20:1) to obtain B-11-3. ¹H NMR (400 MHz, DMSO-d₆) δ ppm 7.75 (d, J=1.6 Hz, 1H), 7.63-7.59 (m, 2H), 7.54-7.50 (m, 2H), 7.19 (dd, J=1.4, 7.9 Hz, 1H), 7.00 (dd, J=1.5, 8.1 Hz, 1H), 6.90-6.83 (m, 1H), 4.27 (d, J=11.1 Hz, 1H), 4.06-4.03 (m, 1H). LCMS: m/z=341.0 [M+H]⁺.

### Step 3: Synthesis of B-11-4

Compound B-11-3 (6 g, 17.57 mmol, 1 eq) was dissolved in EtOH (120 mL), the reaction system was replaced with nitrogen, and sodium borohydride (1.33 g, 35.13 mmol, 2 eq) was added to the reaction flask and stirred for 2 hours at 25° C. The reaction mixture was quenched with saturated aqueous ammonium chloride solution (120 mL) and extracted with ethyl acetate (100 mL*2). The organic phase was collected, washed with saturated aqueous sodium chloride solution (200 mL), dried over anhydrous sodium sulfate, and filtered. The organic phase was concentrated under reduced pressure. The crude product was purified by column chromatography (petroleum ether:ethyl acetate=100:1 to 10:1) to obtain B-11-4. ¹H NMR (400 MHz, DMSO-d₆) δ ppm 9.97-9.68 (m, 1H), 7.48-7.44 (m, 2H), 7.42-7.39 (m, 2H), 7.10-6.95 (m, 1H), 6.87 (d, J=5.0 Hz, 2H), 6.30-6.09 (m, 1H), 4.98 (dd, J=4.6, 7.2 Hz, 1H), 4.06 (dd, J=4.5, 10.0 Hz, 1H), 3.91 (dd, J=7.3, 10.0 Hz, 1H). LCMS: m/z=343.0 [M+H]⁺.

### Step 4: Synthesis of B-11-5

B-11-4 (1 g, 2.91 mmol, 1 eq) and triphenylphosphine (1.53 g, 5.82 mmol, 2 eq) were dissolved in THE (20 mL). The reaction system was replaced with nitrogen, cooled to 0° C., and then a solution of diisopropyl azodicarboxylate (1.18 g, 5.82 mmol, 2 eq) in THE (5 mL) was slowly added dropwise to the reaction flask at 20° C., and the reaction mixture stirred for 10 hours. The reaction mixture was quenched with water (50 mL) and extracted with ethyl acetate (50 mL*3). The organic phase was washed with saturated brine (50 mL), dried over anhydrous sodium sulfate, filtered, and the filtrate was concentrated under reduced pressure to obtain the crude product. The crude product was purified by column chromatography (petroleum ether:ethyl acetate=20:1 to 10:1) to obtain B-11-5. ¹H NMR (400 MHz, DMSO-d₆) δ ppm 7.52 (s, 4H), 7.18 (dd, J=1.4, 8.0 Hz, 1H), 7.01 (dd, J=1.4, 8.1 Hz, 1H), 6.88-6.79 (m, 1H), 5.33 (dd, J=2.4, 8.3 Hz, 1H), 4.57 (dd, J=2.4, 11.5 Hz, 1H), 4.19 (dd, J=8.3, 11.4 Hz, 1H). LCMS: m/z=325.0 [M+H]⁺.

### Step 5: Synthesis of B-11

B-11-5 (720 mg, 2.21 mmol, 1 eq) and compound B-3-7 (2.05 g, 6.63 mmol, 3 eq) were dissolved in 1,4-dioxane (20 mL) and H₂O (4 mL), and then sodium carbonate (703.15 mg, 6.63 mmol, 3 eq) and tetrakis(triphenylphosphine)palladium (255.54 mg, 221.14 μmol, 0.1 eq) were sequentially added thereto. The reaction system was replaced with nitrogen, and the reaction mixture was stirred for 10 hours at 90° C. The reaction mixture was diluted with water (20 mL) and extracted with ethyl acetate (20 mL*3). The resulting organic phase was washed with saturated brine (50 mL), dried over anhydrous sodium sulfate, filtered, and the filtrate was concentrated under reduced pressure to obtain the crude product. The crude product was purified by column chromatography (petroleum ether:ethyl acetate=100:1 to 20:1) to obtain B-11. ¹H NMR (400 MHz, DMSO-d₆) δ ppm 7.50 (s, 4H), 6.92-6.88 (m, 1H), 6.84 (t, J=7.8 Hz, 1H), 6.78-6.74 (m, 1H), 5.92-5.77 (m, 1H), 5.27 (dd, J=2.4, 8.2 Hz, 1H), 4.46 (dd, J=2.5, 11.5 Hz, 1H), 4.10-4.00 (m, 2H), 3.95 (br s, 2H), 3.86 (br s, 1H), 3.53-3.45 (m, 2H), 1.42 (s, 9H). LCMS: m/z=450.2 [M+Na]⁺.

### Reference Example 10: Fragment B-12

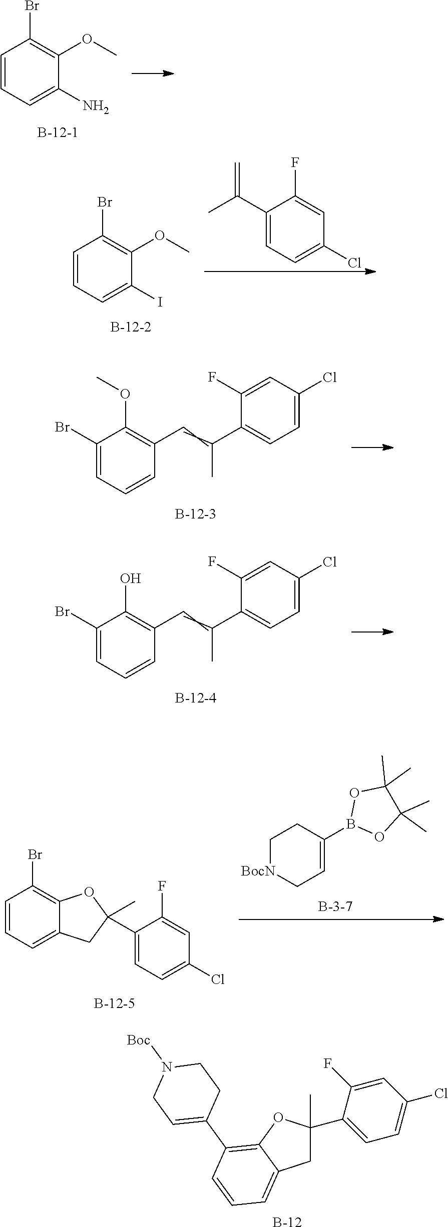

### Step 1: Synthesis of B-12-2

B-12-1 (15 g, 74.24 mmol, 1 eq) was dissolved in water (113 mL) and hydrochloric acid (75 mL), and the mixture was stirred to homogeneity. A solution of sodium nitrite (6.15 g, 89.09 mmol, 1.2 eq) in water (75 mL) was added dropwise to the system at 0° C. and stirred for 0.5 hours until completely dissolved. A solution of potassium iodide (24.65 g, 148.48 mmol, 2 eq) in water (150 mL) was added to the system and stirred for 4 hours at 0° C. 200 mL of ethyl acetate was added thereto, and the phases were separated. The organic phase was washed with saturated sodium sulfite solution (200 mL), dried over anhydrous sodium sulfate, filtered, and the filtrate was concentrated under reduced pressure to obtain the crude product. The crude product was purified by column chromatography (petroleum ether:ethyl acetate=1:0 to 10:1) to obtain B-12-2. LCMS: m/z=313.0 [M+1]⁺.

### Step 2: Synthesis of B-12-3

B-12-2 (20.8 g, 66.47 mmol, 1 eq), 4-chloro-2-fluoro-1-isopropenylbenzene (13.84 g, 81.09 mmol, 1.22 eq), cesium carbonate (64.97 g, 199.41 mmol, 3 eq), and chloro[(tri-tert-butylphosphine)-2-(2-aminobiphenyl)]palladium(II) (3.41 g, 6.65 mmol, 0.1 eq) were dissolved in toluene (125 mL), the reaction system was replaced three times with nitrogen and stirred for 16 hours at 120° C. The system was washed with ethyl acetate (100 mL×3) and water (100 mL), and the organic phase was dried over saturated anhydrous sodium sulfate and concentrated under reduced pressure to obtain the crude product. The crude product was purified by column chromatography (petroleum ether) to obtain B-12-3. LCMS: m/z=355.0 [M+1]⁺.

### Step 3: Synthesis of B-12-4

B-12-3 (2.85 g, 8.01 mmol, 1 eq) was dissolved in DCM (20 mL), then boron tribromide (6.02 g, 24.04 mmol, 2.32 mL, 3 eq) was added thereto, and the reaction mixture was reacted for 2 hours at 20° C. The system was slowly added to 200 mL of water, and the phases were separated. The organic phase was washed with saturated brine (200 mL×3), dried over anhydrous sodium sulfate, filtered, and the resulting filtrate was concentrated under reduced pressure to obtain the crude product of B-12-4. The crude product was directly used in the next step without further purification. LCMS: m/z=341.0 [M+1]⁺.

### Step 4: Synthesis of B-12-5

B-12-4 (2.96 g, 8.67 mmol, 1 eq) was dissolved in formic acid (30 mL) and the reaction mixture was heated to 120° C. and reacted for 16 hours. 40 mL of water and 40 mL of ethyl acetate were added to the system, and the phases were separated. The organic phase was washed with saturated brine, dried over anhydrous sodium sulfate, filtered, and the filtrate was concentrated under reduced pressure to obtain the crude product. The crude product was purified by column chromatography (petroleum ether) to obtain B-12-5. LCMS: m/z=341.0 [M+1]⁺.

### Step 5: Synthesis of B-12

B-12-5 (1.1 g, 3.22 mmol, 1 eq) and B-3-7 (1.05 g, 3.38 mmol, 1.05 eq) were dissolved in 1,4-dioxane (22 mL) and water (2.2 mL), and then [1,1′-bis(diphenylphosphino)ferrocene]dichloropalladium(II) (117.81 mg, 161.00 μmol, 0.05 eq) and potassium carbonate (890.08 mg, 6.44 mmol, 2 eq) were sequentially added thereto. The system was replaced three times with nitrogen, and the reaction was heated to 120° C. and reacted for 16 hours. 30 mL of water and 30 mL of ethyl acetate were added to the system. The resulting organic phase was washed with saturated brine, dried over anhydrous sodium sulfate, filtered, and the filtrate was concentrated under reduced pressure to obtain the crude product. The crude product was purified by column chromatography (petroleum ether:ethyl acetate=1:0 to 5:1) to obtain B-12. LCMS: m/z=444.2 [M+1]⁺.

### Reference Example 11: Fragment B-13

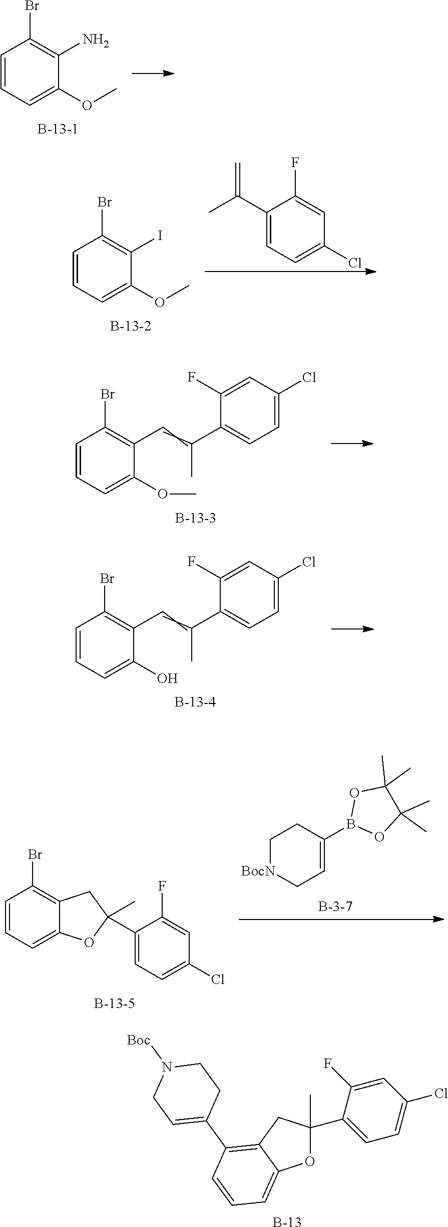

### Step 1: Synthesis of B-13-2

B-13-1 (20 g, 98.99 mmol, 1 eq) was dissolved in water (150 mL) and hydrochloric acid (100 mL), and the mixture was stirred to homogeneity. A solution of sodium nitrite (8.20 g, 118.78 mmol, 1.2 eq) in water (100 mL) was added dropwise to the system at 0° C. and stirred for 0.5 hours until completely dissolved. A solution of potassium iodide (32.86 g, 197.97 mmol, 2 eq) in water (200 mL) was added to the system and stirred for 4 hours at 0° C. 200 mL of ethyl acetate was added thereto, and the phases were separated. The organic phase was washed with saturated sodium sulfite solution (200 mL), dried over anhydrous sodium sulfate, filtered, and the filtrate was concentrated under reduced pressure to obtain the crude product. The crude product was purified by column chromatography (petroleum ether:ethyl acetate=1:0 to 10:1) to obtain B-13-2. LCMS: m/z=313.0 [M+1]⁺.

### Step 2: Synthesis of B-13-3

B-13-2 (26, 83.09 mmol, 1 eq), 4-chloro-2-fluoro-1-isopropenylbenzene (17.29 g, 101.36 mmol, 1.22 eq), bis(triphenylphosphine)palladium(II) chloride (5.83 g, 8.31 mmol, 0.1 eq), and sodium acetate (20.45 g, 249.26 mmol, 3 eq) were dissolved in DMF (420 mL), the reaction system was replaced three times with nitrogen and stirred for 16 hours at 120° C. The system was washed with ethyl acetate (400 mL) and water (400 mL), and the organic phase was dried over saturated anhydrous sodium sulfate and concentrated under reduced pressure to obtain the crude product. The crude product was purified by column chromatography (petroleum ether) to obtain B-13-3. LCMS: m/z=355.0 [M+1]⁺.

### Step 3: Synthesis of B-13-4

B-13-3 (4.65 g, 13.08 mmol, 1 eq) was dissolved in DCM (93 mL), then boron tribromide (9.83 g, 39.23 mmol, 3.78 mL, 3 eq) was added thereto, and the reaction mixture was reacted for 3 hours at 25° C. The system was slowly added to 110 mL of water, and the phases were separated. The organic phase was washed with saturated brine (100 mL×3), dried over anhydrous sodium sulfate, filtered, and the resulting filtrate was concentrated under reduced pressure to obtain the crude product of B-13-4. The system was directly used in the next step without further purification. LCMS: m/z=341.0 [M+1]⁺.

### Step 4: Synthesis of B-13-5

B-13-4 (4.7 g, 13.76 mmol, 1 eq) was dissolved in formic acid (47 mL) and the reaction mixture was heated to 110° C. and reacted for 16 hours. 50 mL of water and 50 mL of ethyl acetate were added to the system, and the pH of the system was adjusted to about 7 with 1 M lithium hydroxide, and the phases were separated. After phase separation, the organic phase was washed with saturated brine, dried over anhydrous sodium sulfate, filtered, the filtrate was concentrated under reduced pressure to obtain the crude product. The crude product was purified by column chromatography (petroleum ether) to obtain B-13-5. LCMS: m/z=341.0 [M+1]⁺.

### Step 5: Synthesis of B-13

B-13-5 (2.9 g, 8.49 mmol, 1 eq) and B-3-7 (2.63 g, 8.49 mmol, 1 eq) were dissolved in 1,4-dioxane (30 mL) and water (3.0 mL), and then [1,1′-bis(diphenylphosphino)ferrocene]dichloropalladium(II) (310.59 mg, 424.50 μmol, 0.05 eq) and potassium carbonate (2.35 g, 16.98 mmol, 2 eq) were sequentially added thereto. The system was replaced three times with nitrogen, and the reaction was heated to 120° C. and reacted for 16 hours. 40 mL of water and 40 mL of ethyl acetate were added to the system. The resulting organic phase was washed with saturated brine, dried over anhydrous sodium sulfate, filtered, and the filtrate was concentrated under reduced pressure to obtain the crude product. The crude product was purified by column chromatography (petroleum ether:ethyl acetate=1:0 to 5:1) to obtain B-13. LCMS: m/z=444.2 [M+1]⁺.

### Example 1

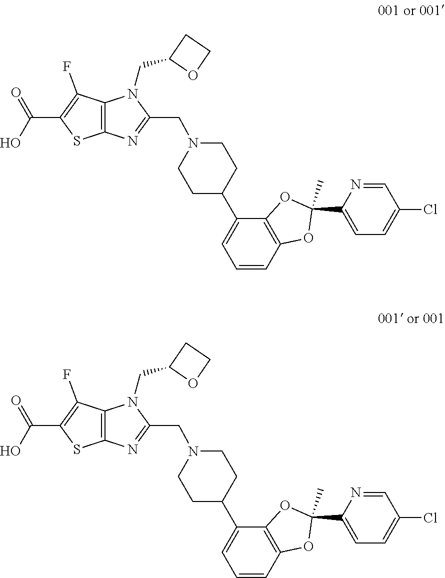 [SMILES: C[C@]1(c2ccc(Cl)cn2)Oc2cccc(C3CCN(Cc4nc5sc(C(=O)O)c(F)c5n4C[C@@H]4CCO4)CC3)c2O1 ; C[C@]1(c2ccc(Cl)cn2)Oc2cccc(C3CCN(Cc4nc5sc(C(=O)O)c(F)c5n4C[C@@H]4CCO4)CC3)c2O1]

### Synthetic Route:

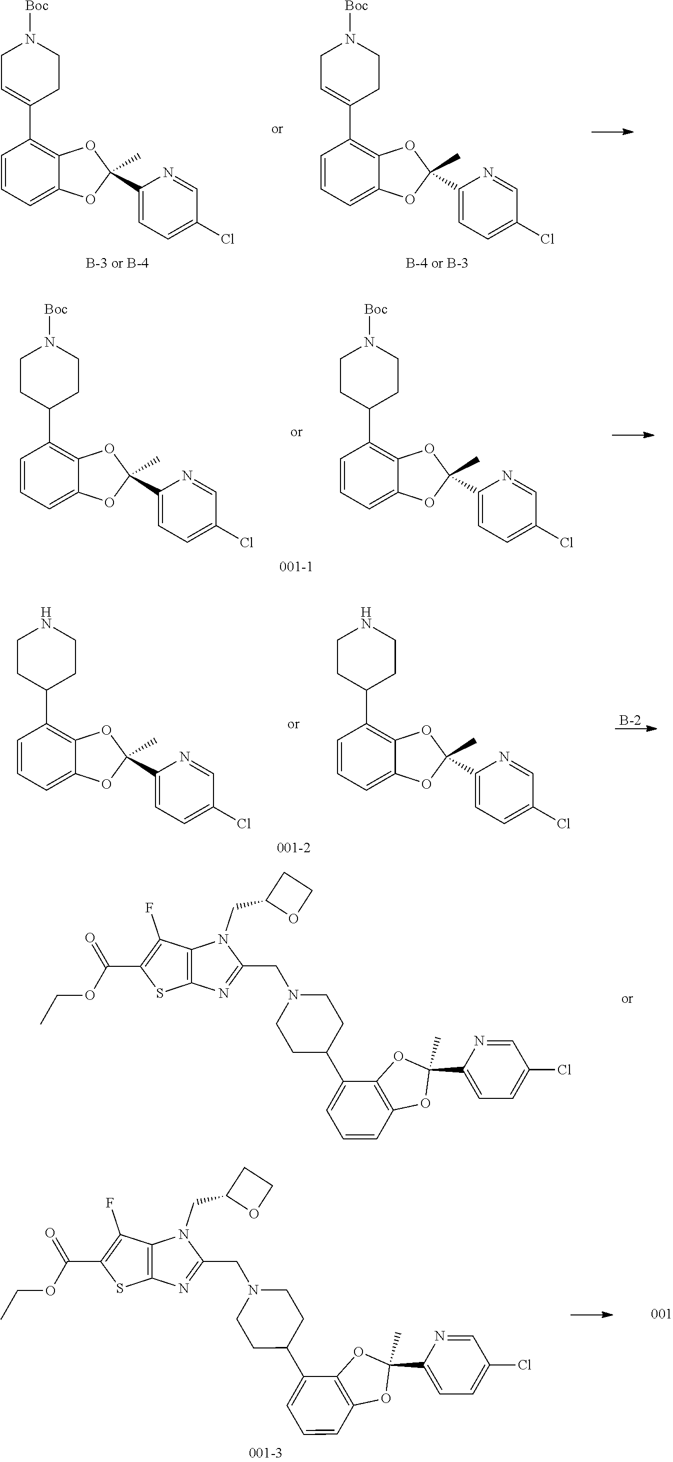

### Step 1: Synthesis of Compound 001-1

B-3 (0.3 g, 699.45 μmol, 1 eq), methanol (6 mL), and tris(triphenylphosphine)rhodium(I) chloride (64.71 mg, 69.95 μmol, 0.1 eq) were sequentially added to a dry reaction flask. The reaction mixture was stirred for 12 hours under hydrogen atmosphere (60° C., 50 psi). The reaction mixture was filtered with diatomite, and the filter cake was washed with methanol (3 mL*3). The resulting filtrate was concentrated to obtain the crude product of 001-1. LCMS: m/z=431.1 [M+1]⁺.

### Step 2: Synthesis of Compound 001-2

Trifluoroacetic acid (2.33 g, 20.42 mmol, 1.51 mL, 20 eq) was added to a solution of 001-1 (440 mg, 1.02 mmol, 1 eq) in dichloromethane (4 mL), and the reaction mixture was reacted for 15 minutes at 25° C. The reaction mixture was directly concentrated to obtain the trifluoroacetate salt of the crude product of 001-2. LCMS: m/z=331.1 [M+1]⁺.

### Step 3: Synthesis of Compound 001-3

001-2 (50.00 mg, crude TFA salt), B-2 (38.54 mg, 120.92 μmol, 0.8 eq), potassium carbonate (83.56 mg, 604.60 μmol, 4.00 eq), and acetonitrile (2 mL) were added to a dry reaction flask, and the reaction mixture was heated to 60° C. and stirred for 10 hours. The reaction mixture was diluted with water (10 mL) and extracted with ethyl acetate (10 mL*3). After phase separation, the organic phase was collected, sequentially washed with saturated brine (10 mL), dried over anhydrous sodium sulfate, filtered, and the resulting filtrate was concentrated under reduced pressure to obtain the crude product of 001-3. LCMS: m/z=627.1 [M+1]⁺.

### Step 4: Synthesis of Compound 001

001-3 (80 mg, 130.48 μmol, 1 eq), methanol (1 mL), tetrahydrofuran (1 mL), water (1 mL), and lithium hydroxide monohydrate (16.43 mg, 391.45 μmol, 3 eq) were sequentially added to a reaction flask, and the reaction mixture was stirred for 6 hours at 20° C. The reaction mixture was concentrated under reduced pressure and purified by preparative high performance liquid chromatography (method: chromatographic column: Waters Xbridge BEH C18 100*30 mm*10 μm; mobile phase: [Water (ammonia water+ammonium bicarbonate)-acetonitrile]; B (acetonitrile) %: 10% to 45%) to obtain compound 001. Chiral analysis SFC detection (instrument: CAS-TJ-ANA-SFC-G (Waters UPCC with PDA); chromatographic column: Chiralcel OJ-3, 50×4.6 mm I.D., 3 μm; mobile phase A: supercritical carbon dioxide, B: methanol (0.1% isopropylamine); gradient: the content of B increased from 5% to 50% in 0.2 minutes and held for 2 minutes, and then decreased from 50% to 5% in 2.2 minutes) showed a retention time of 1.422 minutes for compound 001 with an ee value of 100%. ¹H NMR (400 MHz, CD₃OD) δ ppm ppm 8.67-8.56 (m, 1H), 7.94-7.87 (m, 1H), 7.68 (br d, J=8.4 Hz, 1H), 6.87-6.72 (m, 3H), 5.27-5.22 (m, 2H), 4.74-4.68 (m, 3H), 4.50-4.43 (m, 1H), 4.15-4.07 (m, 2H), 3.35-3.31 (m, 1H), 3.27-3.20 (m, 1H), 2.87-2.80 (m, 2H), 2.64-2.50 (m, 3H), 2.07-1.92 (m, 6H). LCMS: m/z=599.1 [M+1]⁺.

Using compound B-4 as raw material, compound 001′ was synthesized by referring to the synthesis method of steps 1 to 4 of Example 1. Chiral analysis SFC detection (instrument: CAS-TJ-ANA-SFC-G (Waters UPCC with PDA); chromatographic column: Chiralcel OJ-3, 50×4.6 mm I.D., 3 μm; mobile phase A: supercritical carbon dioxide, B: methanol (0.1% isopropylamine); gradient: the content of B increased from 5% to 50% in 0.2 minutes and held for 2 minutes, and then decreased from 50% to 5% in 2.2 minutes) showed a retention time of 1.030 minutes for compound 001′ with an ee value of 100%. ¹H NMR (400 MHz, CD₃OD) δ ppm 8.72-8.51 (m, 1H), 8.00-7.79 (m, 1H), 7.66 (d, J=8.5 Hz, 1H), 6.91-6.66 (m, 4H), 5.29-5.15 (m, 2H), 4.50-4.35 (m, 3H), 4.22-4.06 (m, 3H), 3.77 (s, 1H), 3.64 (s, 1H), 2.94-2.74 (m, 3H), 2.71-2.57 (m, 3H), 2.54-2.42 (m, 1H), 1.97-1.85 (m, 3H). LCMS: m/z=599.1 [M+1]⁺.

### Example 2

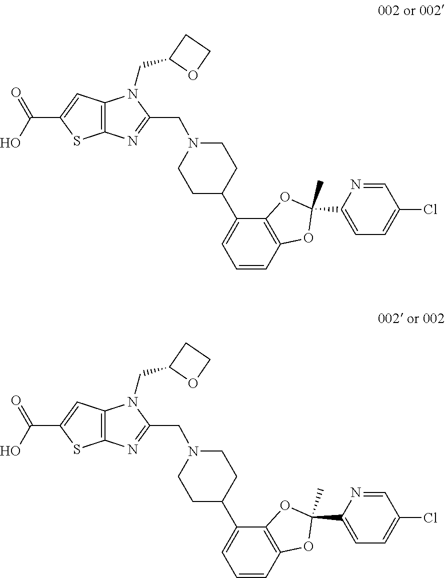 [SMILES: C[C@@]1(c2ccc(Cl)cn2)Oc2cccc(C3CCN(Cc4nc5sc(C(=O)O)cc5n4C[C@@H]4CCO4)CC3)c2O1 ; C[C@]1(c2ccc(Cl)cn2)Oc2cccc(C3CCN(Cc4nc5sc(C(=O)O)cc5n4C[C@@H]4CCO4)CC3)c2O1]

### Synthetic Route:

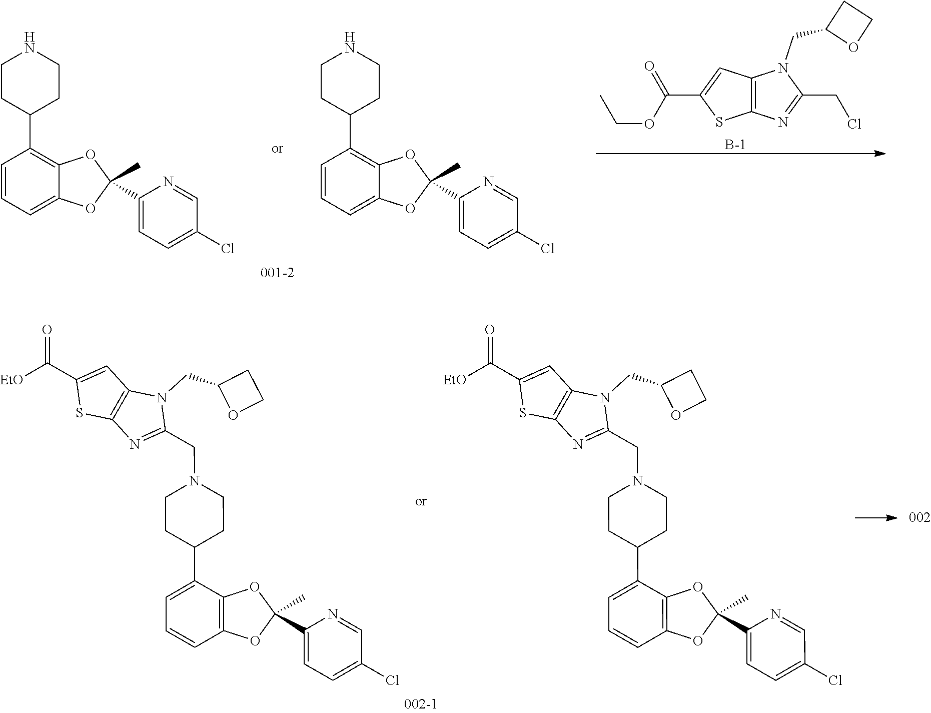

### Step 1: Synthesis of Compound 002-1

001-2 (46.00 mg, 139.05 μmol, 1 eq), B-1 (43.77 mg, 139.05 μmol, 1 eq), potassium carbonate (76.87 mg, 556.21 μmol, 4 eq), and acetonitrile (2 mL) were added to a dry reaction flask, and the reaction mixture was heated to 60° C. and stirred for 10 hours. The reaction mixture was diluted with water (10 mL) and extracted with ethyl acetate (3*10 mL). After phase separation, the organic phase was collected, sequentially washed with saturated brine (10 mL), dried over anhydrous sodium sulfate, filtered, and the resulting filtrate was concentrated under reduced pressure to obtain the crude product. The crude product was purified by preparative thin-layer chromatography (PE:EA=1:0 to 2:1) to obtain 002-1. LCMS: m/z=609.2 [M+1]⁺.

### Step 2: Synthesis of Compound 002

002-1 (27.00 mg, 44.33 μmol, 1 eq), methanol (0.5 mL), tetrahydrofuran (0.5 mL), water (0.5 mL), and lithium hydroxide monohydrate (5.58 mg, 132.98 μmol, 3 eq) were sequentially added to a dry reaction flask, and the reaction system was replaced with nitrogen and stirred for 6 hours at 20° C. The reaction mixture was concentrated under reduced pressure and purified by preparative high performance liquid chromatography (method: chromatographic column: Waters Xbridge BEH C18 100*30 mm*10 m; mobile phase: [Water (ammonium bicarbonate)-acetonitrile]; B(acetonitrile) %: 15% to 45%) to obtain compound 002. Chiral analysis SFC detection (instrument: CAS-TJ-ANA-SFC—H (Waters UPCC with SQ Detector 2); chromatographic column: Chiralpak AD-3, 50×4.6 mm I.D., 3 μm; mobile phase A: supercritical carbon dioxide, B: methanol (0.1% isopropylamine); gradient: the content of B increased from 5% to 50% in 0.2 minutes and held for 2 minutes, and then decreased from 50% to 5% in 2.2 minutes) showed a retention time of 1.264 minutes for compound 002 with an ee value of 100%. ¹H NMR (400 MHz, CD₃OD) δ ppm 8.60 (d, J=1.9 Hz, 1H), 7.69-7.65 (m, 1H), 7.61-7.58 (m, 1H), 7.42 (s, 1H), 6.81-6.67 (m, 3H), 5.17-5.04 (m, 1H), 4.67-4.52 (m, 3H), 4.39-4.33 (m, 1H), 4.15-4.01 (m, 2H), 3.57-3.42 (m, 2H), 2.88-2.79 (m, 1H), 2.73-2.65 (m, 1H), 2.60-2.51 (m, 2H), 2.43-2.37 (m, 1H), 2.11 (br s, 1H), 2.03 (s, 3H), 1.93 (br s, 2H), 1.65 (s, 1H). LCMS: m/z=581.1 [M+1]⁺.

Using compound B-4 as raw material, compound 002′ was synthesized by referring to the synthesis methods of steps 1 to 2 in Example 1 and steps 1 to 2 of Example 2. Chiral analysis SFC detection (instrument: CAS-TJ-ANA-SFC—H (Waters UPCC with SQ Detector 2); chromatographic column: Chiralpak AD-3, 50×4.6 mm I.D., 3 μm; mobile phase A: supercritical carbon dioxide, B: methanol (0.1% isopropylamine); gradient: the content of B increased from 5% to 50% in 0.2 minutes and held for 2 minutes, and then decreased from 50% to 5% in 2.2 minutes) showed a retention time of 1.246 minutes for compound 002′ with an ee value of 100%. ¹H NMR (400 MHz, CD₃OD) δ ppm 8.68 (d, J=2.4 Hz, 1H), 7.97-7.92 (m, 1H), 7.78-7.70 (m, 2H), 6.93-6.85 (m, 1H), 6.79-6.78 (m, 1H), 6.82-6.78 (m, 1H), 5.38-5.20 (m, 1H), 4.79-4.61 (m, 4H), 4.53-4.47 (m, 1H), 4.27 (br d, J=2.1 Hz, 2H), 3.46 (br d, J=11.6 Hz, 1H), 2.94-2.75 (m, 4H), 2.62-2.53 (m, 1H), 2.09 (s, 4H), 1.99-1.98 (m, 1H), 2.04-1.96 (m, 2H). LCMS: m/z=581.1 [M+1]⁺.

### Example 3

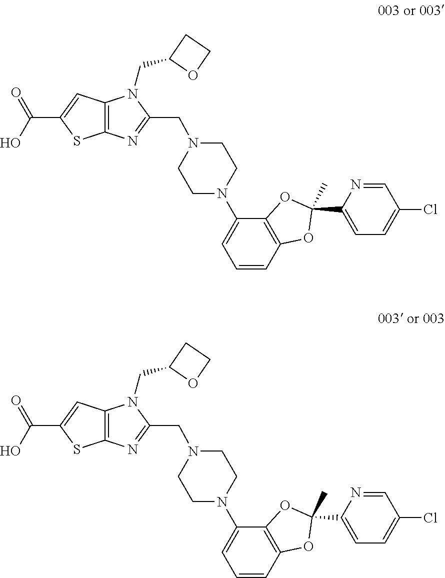 [SMILES: C[C@]1(c2ccc(Cl)cn2)Oc2cccc(N3CCN(Cc4nc5sc(C(=O)O)cc5n4C[C@@H]4CCO4)CC3)c2O1 ; C[C@@]1(c2ccc(Cl)cn2)Oc2cccc(N3CCN(Cc4nc5sc(C(=O)O)cc5n4C[C@@H]4CCO4)CC3)c2O1]

### Synthetic Route:

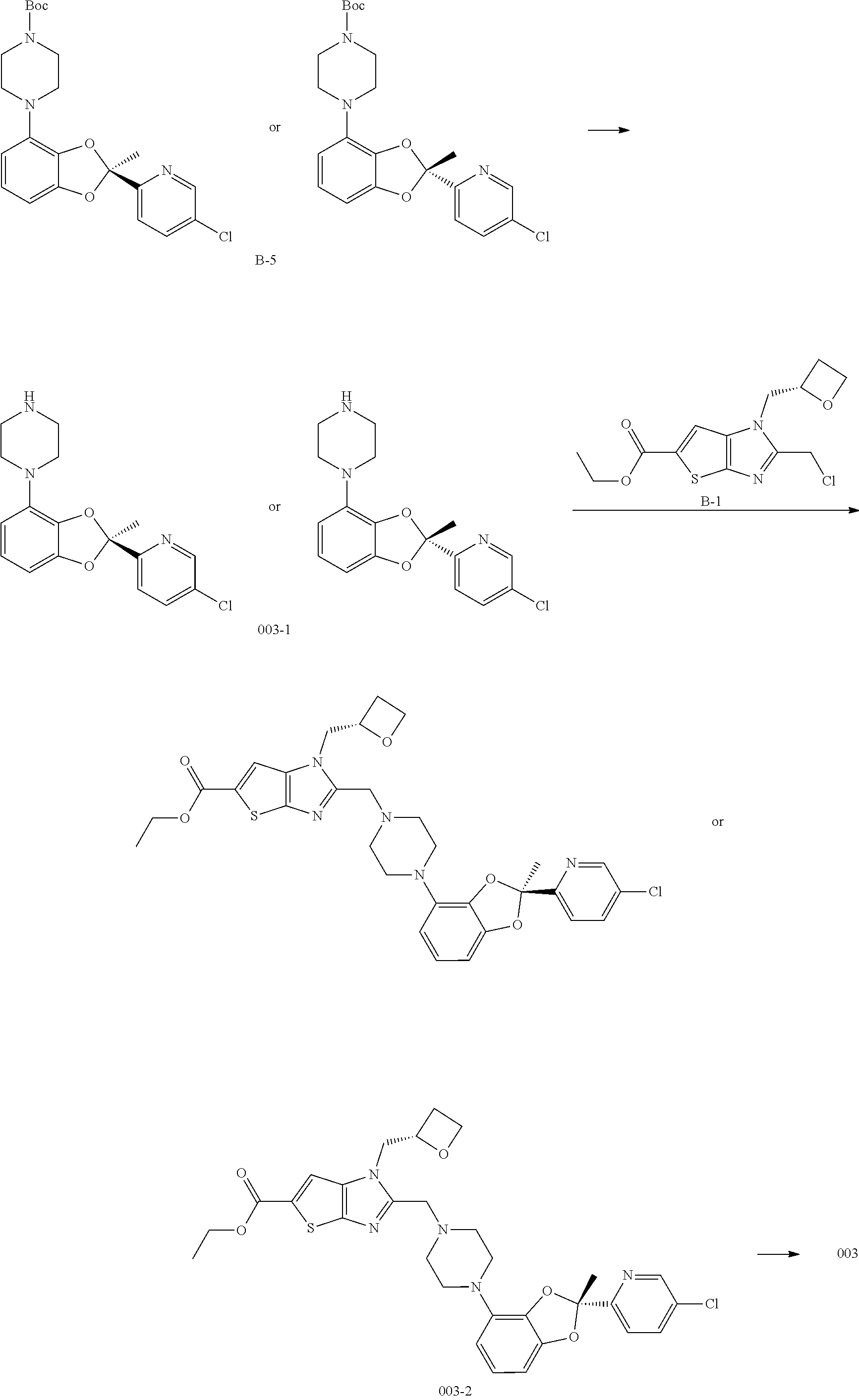

### Step 1: Synthesis of Compound 003-1

B-5 (100 mg, 231.53 μmol, 1 eq) was dissolved in dichloromethane (1 mL), then trifluoroacetic acid (0.2 mL) was added thereto, and the reaction mixture was stirred for 1 hour at 20° C. The reaction mixture was directly concentrated under reduced pressure to obtain crude trifluoroacetate salt of 003-1. LCMS: m/z=332.1 [M+1]⁺.

### Step 2: Synthesis of Compound 003-2

003-1 (77.00 mg, crude trifluoroacetate salt), B-1 (54.37 mg, 172.72 μmol, 1 eq), potassium carbonate (143.23 mg, 1.04 mmol, 6 eq), and acetonitrile (2 mL) were sequentially added to a dry reaction flask, and the reaction mixture was heated to 60° C. and stirred for 10 hours. The reaction mixture was added with water (10 mL) and extracted with ethyl acetate (10 mL*3). The organic phase was collected after phase separation, washed with saturated brine (10 mL*3), then dried over anhydrous sodium sulfate, and the resulting filtrate was concentrated under reduced pressure to obtain the crude product. The crude product was purified by thin-layer chromatography silica gel plate (petroleum ether:ethyl acetate=2:1) to obtain 003-2. LCMS: m/z=610.2 [M+1]⁺.

### Step 3: Synthesis of Compound 003

003-2 (60 mg, 98.34 μmol, 1 eq) was dissolved in tetrahydrofuran (0.5 mL), methanol (0.5 mL), and water (0.5 mL), then lithium hydroxide monohydrate (12.38 mg, 295.02 μmol, 3 eq) was added thereto, and the reaction mixture was stirred for 6 hours at 20° C. The reaction mixture was concentrated under reduced pressure to obtain the crude product, which was purified by preparative high performance liquid chromatography to obtain 003. Method: chromatographic column: Waters Xbridge BEH C18 100*30 mm*10 m; mobile phase: [Water (ammonium bicarbonate)-acetonitrile]; B (acetonitrile) %: 30% to 60%. Chiral analysis SFC detection (instrument: CAS-TJ-ANA-SFC—H (Waters UPCC with SQ Detector 2); chromatographic column: Chiralpak IG-3, 50×4.6 mm I.D., 3 μm; mobile phase A: supercritical carbon dioxide, B: methanol (0.1% isopropylamine); gradient: the content of B increased from 5% to 50% in 0.2 minutes and held for 1 minute, and then decreased from 50% to 5% in 2.2 minutes) showed a retention time of 1.856 minutes with an ee value of 100%. ¹H NMR (400 MHz, CD₃OD) δ ppm 8.59 (d, J=2.4 Hz, 1H), 7.87 (dd, J=2.4, 8.5 Hz, 1H), 7.79 (s, 1H), 7.66 (dd, J=0.6, 8.5 Hz, 1H), 6.83-6.71 (m, 1H), 6.56-6.41 (m, 2H), 5.22 (br dd, J=2.6, 7.4 Hz, 1H), 4.74-4.64 (m, 2H), 4.60-4.54 (m, 1H), 4.43 (td, J=6.0, 9.2 Hz, 1H), 4.02-3.80 (m, 2H), 3.20 (br s, 4H), 2.83-2.75 (m, 1H), 2.74-2.63 (m, 4H), 2.48 (tdd, J=7.2, 9.1, 11.4 Hz, 1H), 2.02 (s, 3H). LCMS: m/z=582.1 [M+1]⁺.

Using compound B-6 as raw material, compound 003′ was synthesized by referring to the synthesis method of steps 1 to 3 of Example 3. Chiral analysis SFC detection (instrument: CAS-TJ-ANA-SFC—H (Waters UPCC with SQ Detector 2); chromatographic column: Chiralpak IG-3, 50×4.6 mm I.D., 3 μm; mobile phase A: supercritical carbon dioxide, B: methanol (0.1% isopropylamine); gradient: the content of B increased from 5% to 50% in 0.2 minutes and held for 1 minute, and then decreased from 50% to 5% in 2.2 minutes) showed a retention time of 1.730 minutes with an ee value of 100%. ¹H NMR (400 MHz, CD₃OD) δ ppm 8.59 (d, J=2.4 Hz, 1H), 7.87 (dd, J=2.4, 8.4 Hz, 1H), 7.78 (s, 1H), 7.66 (d, J=8.5 Hz, 1H), 6.83-6.70 (m, 1H), 6.49 (dd, J=8.0, 13.3 Hz, 2H), 5.22 (dq, J=2.8, 7.1 Hz, 1H), 4.72-4.63 (m, 2H), 4.61-4.55 (m, 1H), 4.43 (td, J=5.9, 9.1 Hz, 1H), 3.99-3.82 (m, 2H), 3.19 (br s, 4H), 2.83-2.75 (m, 1H), 2.71 (br d, J=4.4 Hz, 4H), 2.48 (tdd, J=7.2, 9.1, 11.3 Hz, 1H), 2.02 (s, 3H). LCMS: m/z=581.1 [M+1]⁺.

### Example 4

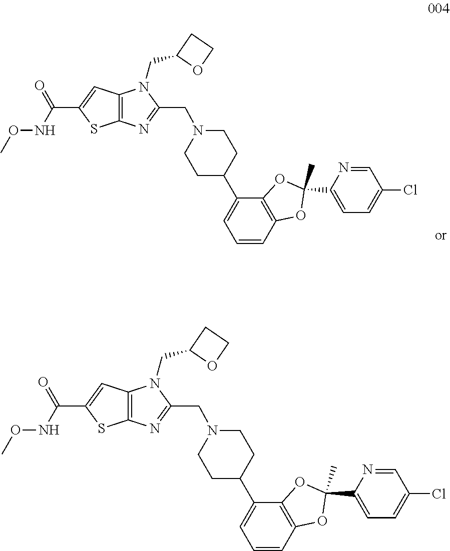 [SMILES: CONC(=O)c1cc2c(nc(CN3CCC(c4cccc5c4O[C@](C)(c4ccc(Cl)cn4)O5)CC3)n2C[C@@H]2CCO2)s1 ; CONC(=O)c1cc2c(nc(CN3CCC(c4cccc5c4O[C@@](C)(c4ccc(Cl)cn4)O5)CC3)n2C[C@@H]2CCO2)s1]

### Synthetic Route:

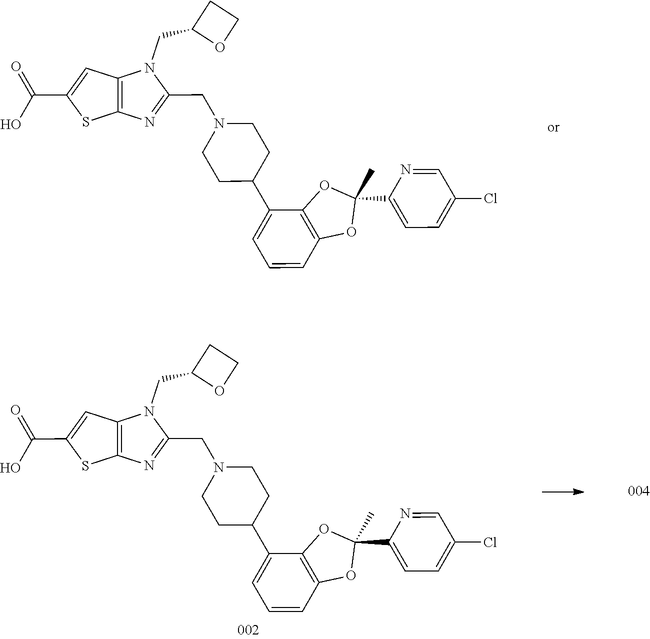 [SMILES: C[C@@]1(c2ccc(Cl)cn2)Oc2cccc(C3CCN(Cc4nc5sc(C(=O)O)cc5n4C[C@@H]4CCO4)CC3)c2O1 ; "or" ; C[C@]1(c2ccc(Cl)cn2)Oc2cccc(C3CCN(Cc4nc5sc(C(=O)O)cc5n4C[C@@H]4CCO4)CC3)c2O1]

002 (150 mg, 0.26 mmol, 1 eq), dichloromethane (12.0 mL), HATU (147.2 mg, 0.39 mmol, 1.5 eq), and DIPEA (157.6 μL, 0.91 mmol, 3.5 eq) were sequentially added to a dry reaction flask, and the reaction mixture was stirred at 24° C. for 0.5 hours. Methoxyamine hydrochloride (32.4 mg, 0.26 mmol, 1.5 eq) was then added thereto, and the reaction mixture was stirred for another 0.5 hours at 24° C. The reaction mixture was added with water (10 mL) and extracted with dichloromethane (10 mL*3). The organic phase was collected after phase separation, washed with saturated brine (10 mL*2), and dried over anhydrous sodium sulfate, and the resulting filtrate was concentrated under reduced pressure to obtain the crude product. The crude product was purified by thin-layer chromatography silica gel plate (dichloromethane:methanol=20:1) and preparative high performance liquid chromatography, method: chromatographic column: Waters Xbridge BEH C18 100*30 mm*10 m; mobile phase: [water (ammonium bicarbonate)-acetonitrile]; gradient (acetonitrile) %: 30% to 60%, to obtain 004. ¹H NMR (400 MHz, CDCl₃) δ ppm 8.61-8.53 (m, 1H), 7.70-7.62 (m, 2H), 7.58-7.52 (m, 1H), 6.79-6.72 (m, 1H), 6.71-6.63 (m, 2H), 5.30 (s, 3H), 5.22-5.12 (m, 1H), 4.64-4.57 (m, 1H), 4.55-4.44 (m, 2H), 4.42-4.34 (m, 1H), 3.82 (s, 3H), 3.79-3.74 (m, 1H), 3.62-3.54 (m, 2H), 3.06-3.02 (m, 2H), 2.97-2.93 (m, 1H), 2.99-2.92 (m, 1H), 2.91-2.82 (m, 1H), 2.78-2.66 (m, 2H), 2.49-2.41 (m, 1H), 2.30-2.12 (m, 2H). LCMS: m/z=610.2 [M+1]⁺.

### Example 5

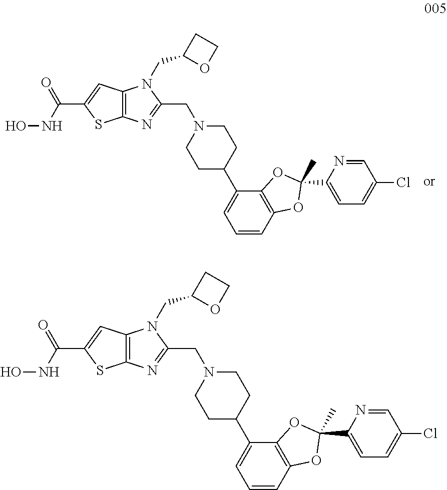 [SMILES: C[C@@]1(c2ccc(Cl)cn2)Oc2cccc(C3CCN(Cc4nc5sc(C(=O)NO)cc5n4C[C@@H]4CCO4)CC3)c2O1 ; "or" ; C[C@]1(c2ccc(Cl)cn2)Oc2cccc(C3CCN(Cc4nc5sc(C(=O)NO)cc5n4C[C@@H]4CCO4)CC3)c2O1]

### Synthetic Route:

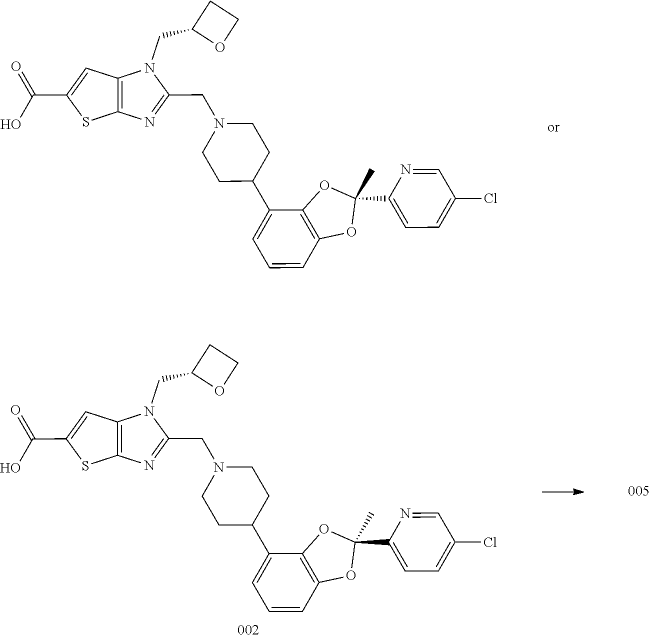 [SMILES: C[C@@]1(c2ccc(Cl)cn2)Oc2cccc(C3CCN(Cc4nc5sc(C(=O)O)cc5n4C[C@@H]4CCO4)CC3)c2O1 ; "or" ; C[C@]1(c2ccc(Cl)cn2)Oc2cccc(C3CCN(Cc4nc5sc(C(=O)O)cc5n4C[C@@H]4CCO4)CC3)c2O1]

002 (145.8 mg, 0.24 mmol, 1 eq), dichloromethane (12.0 mL), HATU (139.5 mg, 0.36 mmol, 1.5 eq), and DIPEA (277.0 μL, 1.59 mmol, 6.5 eq) were sequentially added to a dry reaction flask, and the reaction mixture was stirred at 24° C. for 0.5 hours. Hydroxylamine hydrochloride (25.5 mg, 0.24 mmol, 1.5 eq) was then added thereto, and the reaction mixture was stirred for another 0.5 hours at 24° C. The reaction mixture was added with water (10 mL) and extracted with dichloromethane (10 mL*3). The organic phase was collected after phase separation, washed with saturated brine (10 mL*2), and dried over anhydrous sodium sulfate, and the resulting filtrate was concentrated under reduced pressure to obtain the crude product. The crude product was purified by thin-layer chromatography silica gel plate (dichloromethane:methanol=10:1) and preparative high performance liquid chromatography, method: chromatographic column: Waters Xbridge BEH C18 100*30 mm*10 m; mobile phase: [water (ammonium bicarbonate)-acetonitrile]; gradient (acetonitrile) %: 30% to 60%, to obtain 005. ¹H NMR (400 MHz, CDCl₃) δ ppm 8.62 (d, J=2.0 Hz, 1H), 7.72 (dd, J=2.0, 8.0 Hz, 1H), 7.65 (dd, J=2.0, 8.0 Hz, 1H), 7.31 (s, 1H), 6.80 (q, J=8.0 Hz, 1H), 6.75 (q, J=8.0 Hz, 2H), 5.22-5.12 (m, 1H), 4.90-4.82 (m, 1H), 4.70-4.60 (m, 2H), 4.41-4.31 (m, 2H), 2.95-2.88 (m, 1H), 2.78-2.74 (m, 1H), 2.52-2.47 (m, 1H), 2.32-2.17 (m, 2H), 2.08 (s, 3H), 2.01-1.96 (m, 2H), 1.62-1.47 (m, 3H), 1.20-1.17 (m, 2H).

### Example 6

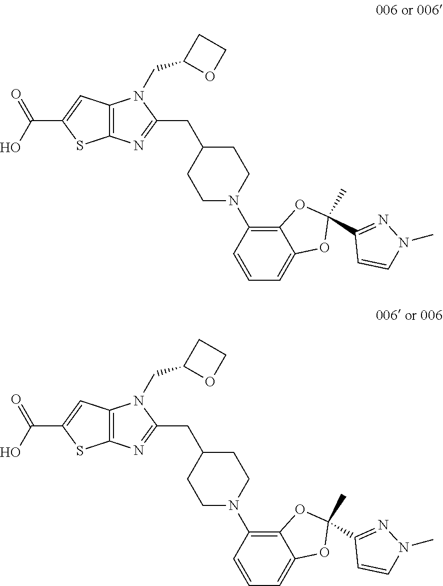 [SMILES: Cn1ccc([C@@]2(C)Oc3cccc(N4CCC(Cc5nc6sc(C(=O)O)cc6n5C[C@@H]5CCO5)CC4)c3O2)n1 ; Cn1ccc([C@]2(C)Oc3cccc(N4CCC(Cc5nc6sc(C(=O)O)cc6n5C[C@@H]5CCO5)CC4)c3O2)n1]

### Synthetic Route:

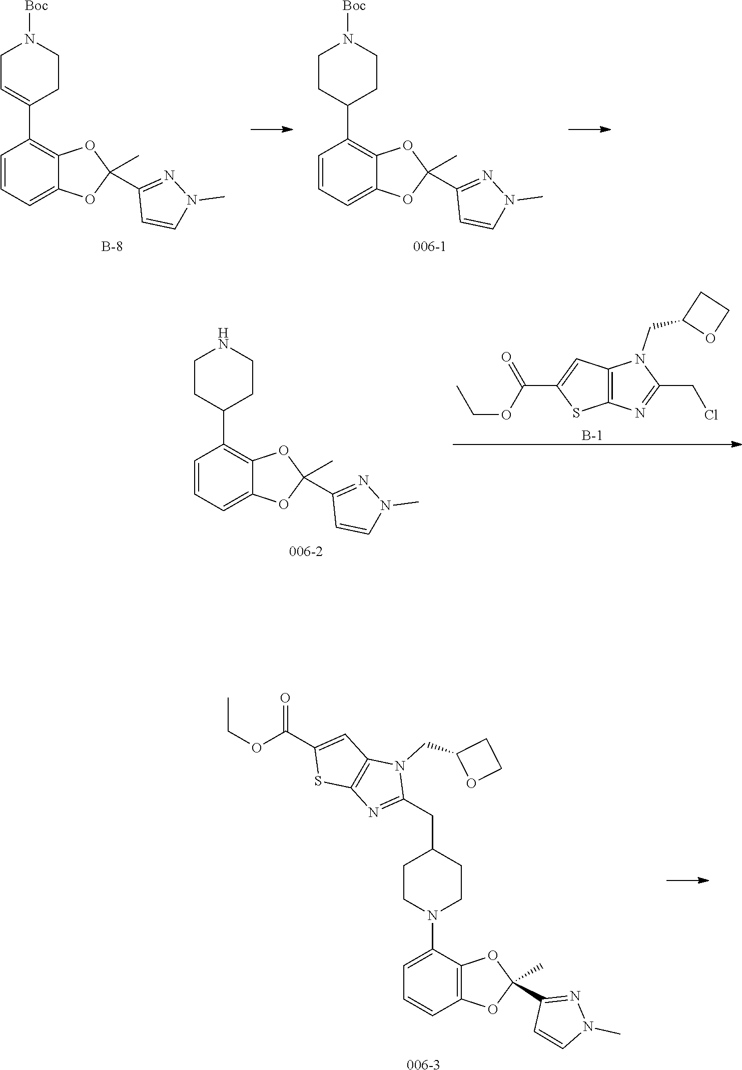

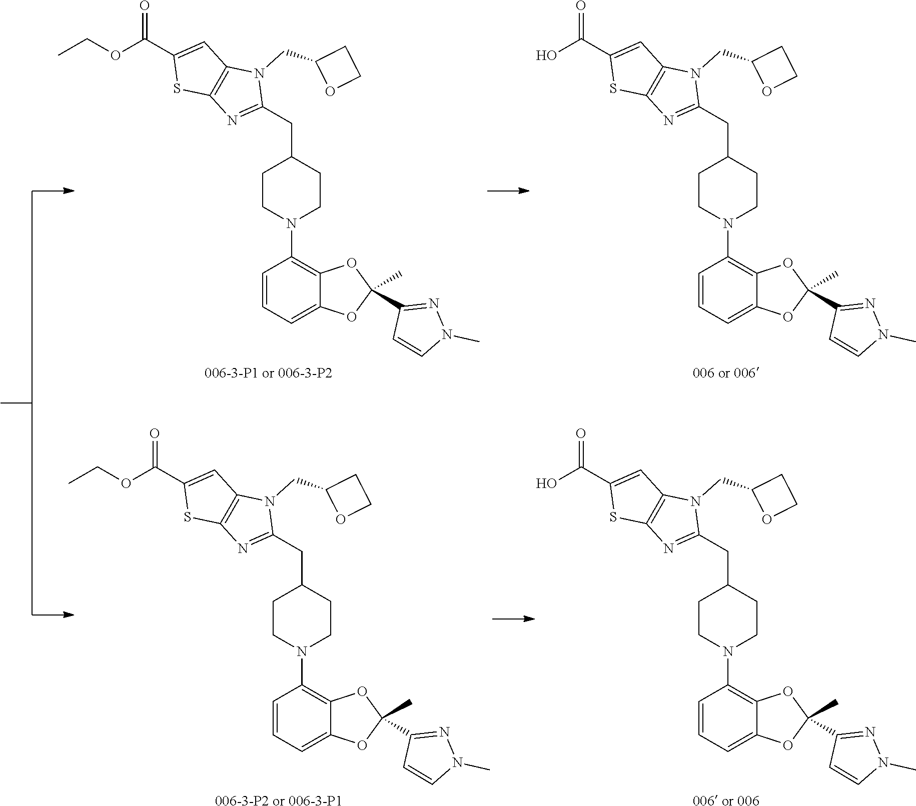

### Step 1: Synthesis of Compound 006-1

Pd/C (5.0 g, 13.21 mmol, a content of 10%, 1 eq) was added to a reaction flask under argon atmosphere, then B-8 (5.25 g, 13.21 mmol, 1 eq) and 60 mL of methanol were sequentially added thereto, and the reaction system was stirred for 12 hours under hydrogen atmosphere (50° C., 45 psi). The reaction mixture was filtered and the filtrate was concentrated. The crude product was purified by column chromatography (petroleum ether:ethyl acetate=1:0 to 4:1) to obtain 006-1. LCMS: m/z=400.2 [M+1]⁺.

### Step 2: Synthesis of Compound 006-2

006-1 (1.0 g, 2.50 mmol, 1 eq) was dissolved in 10 mL of dichloromethane, then trifluoroacetic acid (25.03 mmol, 1.85 mL, 10 eq) was added dropwise thereto, and the reaction mixture was stirred for 2 hours at 24° C. The reaction mixture was added with saturated sodium bicarbonate to adjust the pH of the reaction mixture to about 7, and the reaction mixture was extracted with dichloromethane (50 mL*2). The organic phases were combined, washed with 20 mL of water, dried over anhydrous sodium sulfate, and then filtered, and the filtrate was concentrated under reduced pressure to obtain a crude product. The crude product was purified by column chromatography (dichloromethane:methanol=1:0 to 10:1) to obtain 006-2. LCMS: m/z=299.9 [M+1]⁺.

### Step 3: Synthesis of Compound 006-3

006-2 (0.1 g, 0.33 mmol, 1 eq) and B-1 (95.0 mg, 0.3 mmol, 0.9 eq) were dissolved in 1 mL of acetonitrile, then potassium carbonate (0.18 g, 1.34 mmol, 4 eq) was added thereto, and the reaction mixture was heated to 60° C. and stirred for 12 hours. Acetonitrile was removed by rotary evaporation under reduced pressure, and the reaction mixture was then diluted with 10 mL of water, and extracted with ethyl acetate (30 mL*2). The organic phases were combined, dried over anhydrous sodium sulfate, filtered, and the filtrate was concentrated under reduced pressure to obtain the crude product. The crude product was purified by column chromatography (dichloromethane:methanol=1:0 to 10:1) to obtain 006-3.

Step 4: Synthesis of compounds 006-3-P1 and 006-3-P2

006-3 was purified by preparative SFC (column model: DAICEL CHIRALPAK IC (250 mm*30 mm*10 m); mobile phase: phase A was supercritical carbon dioxide, phase B was ethanol (0.2% ammonia water); gradient: 35% to 35%) to obtain compound 006-3-P1 and compound 006-3-P2, which were subjected to chiral analysis SFC detection (column model: Chiralpak AD-3, 50×4.6 mm I.D., 3 m); mobile phase: phase A was supercritical carbon dioxide, phase B was ethanol (0.2% ammonia water); gradient (B %): 5% to 35%), showing that the Rt for compound 006-3-P1 was 0.872 minutes, with an ee value of 100%, and the Rt for compound 006-3-P2 was 1.197 minutes, with an ee value of 98.4%. LCMS: m/z=578.0 [M+1]⁺.

### Step 5: Synthesis of Compounds 006 and 006′

006-3-P1 (0.12 g, 0.21 mmol, 1 eq), methanol (0.5 mL), tetrahydrofuran (0.5 mL), water (0.5 mL), and lithium hydroxide monohydrate (26.2 mg, 0.62 mmol, 3 eq) were sequentially added to a dry reaction flask, and the reaction system was replaced with nitrogen and stirred for 3 hours at 28° C. The reaction mixture was concentrated under reduced pressure and purified by high performance liquid chromatography (method: chromatographic column: Phenomenex C18 75*30 mm*3 μm; mobile phase: [water (ammonium bicarbonate)-acetonitrile]; gradient (acetonitrile) %: 30% to 60%) to obtain 006. ¹H NMR (400 MHz, CDCl₃) δ ppm 8.80 (br s, 1H), 7.36-7.61 (m, 2H), 6.62-6.80 (m, 3H), 5.07 (br s, 1H), 4.53 (br s, 2H), 4.32 (br s, 1H), 3.94 (s, 3H), 3.86-4.14 (m, 2H), 3.39 (br s, 4H), 2.78 (br s, 1H), 2.63 (br s, 1H), 2.32-2.51 (m, 2H), 2.08 (s, 3H), 1.94 (br s, 4H). LCMS: m/z=550.3 [M+1]⁺.

Using compound 006-3-P2 as raw material, compound 006′ was synthesized by referring to the synthesis method of step 5 of Example 6. ¹H NMR (400 MHz, CDCl₃) δ ppm 8.81 (br s, 1H), 7.36-7.61 (m, 2H), 6.62-6.80 (m, 3H), 5.05 (br s, 1H), 4.53 (br s, 2H), 4.32 (br s, 1H), 3.94 (s, 3H), 3.86-4.15 (m, 2H), 3.39 (br s, 4H), 2.76 (br s, 1H), 2.61 (br s, 1H), 2.32-2.51 (m, 2H), 2.06 (s, 3H), 1.92 (br s, 4H). LCMS: m/z=550.3 [M+1]⁺.

### Example 7

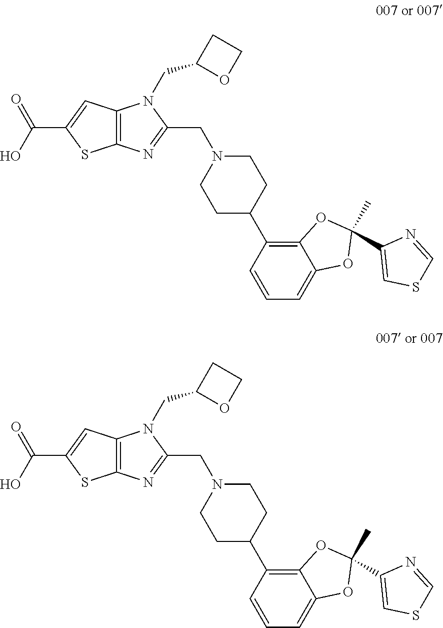 [SMILES: C[C@]1(c2cscn2)Oc2cccc(C3CCN(Cc4nc5sc(C(=O)O)cc5n4C[C@@H]4CCO4)CC3)c2O1 ; C[C@@]1(c2cscn2)Oc2cccc(C3CCN(Cc4nc5sc(C(=O)O)cc5n4C[C@@H]4CCO4)CC3)c2O1]

### Synthetic Route:

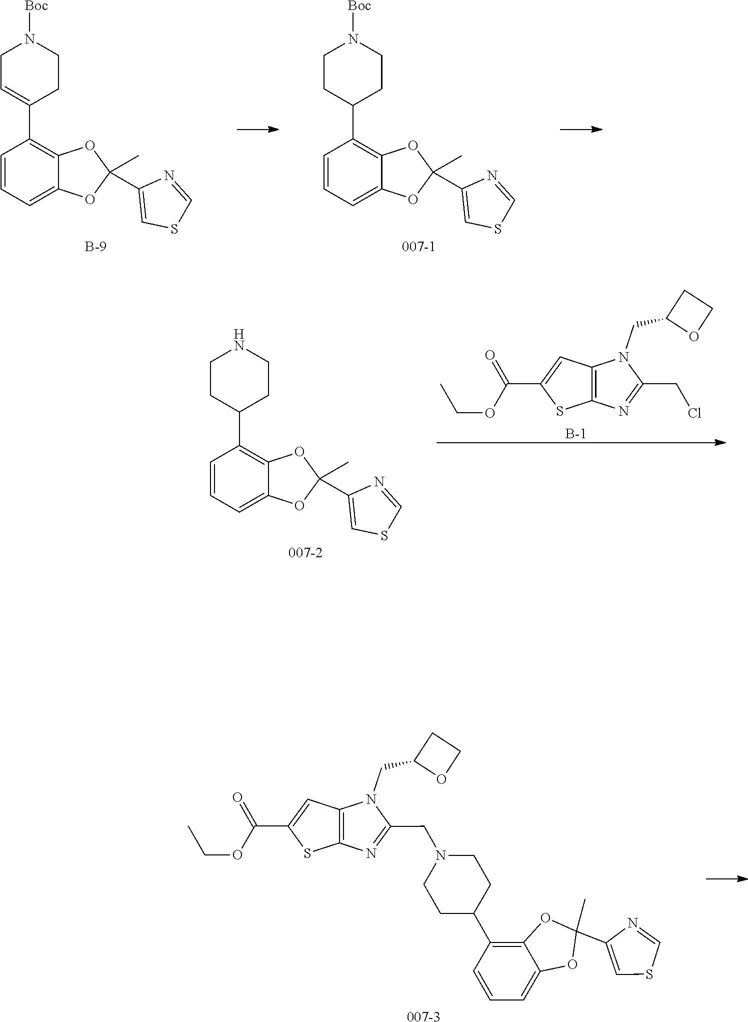

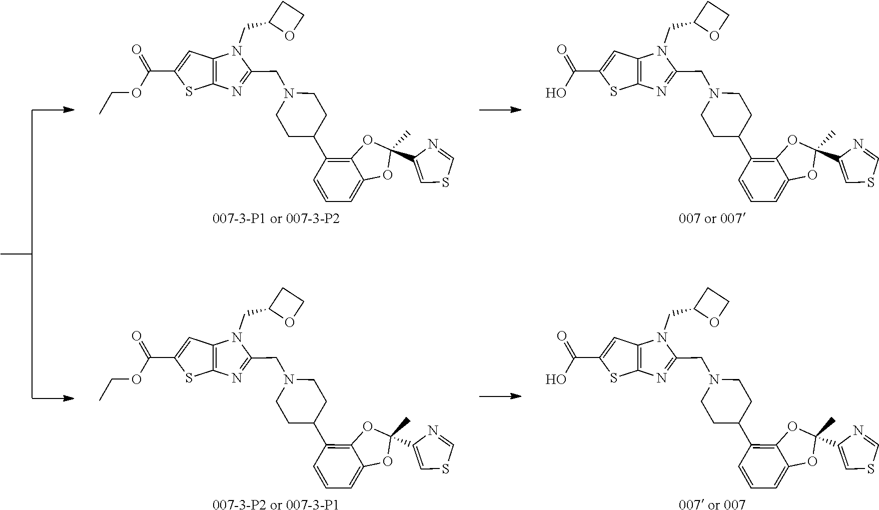

### Step 1: Synthesis of Compound 007-1

Pd/C (5.0 g, 13.21 mmol, a content of 10%, 1 eq) was added to a reaction flask under argon atmosphere, then B-9 (5.29 g, 13.21 mmol, 1 eq) and 60 mL of methanol were sequentially added thereto, and the reaction system was stirred for 12 hours under hydrogen atmosphere (50° C., 45 psi). The reaction mixture was filtered and the filtrate was concentrated. The crude product was purified by column chromatography (petroleum ether:ethyl acetate=1:0 to 6:1) to obtain 007-1. LCMS: m/z=403.2 [M+1]⁺.

### Step 2: Synthesis of Compound 007-2

007-1 (1.0 g, 2.50 mmol, 1 eq) was dissolved in 10 mL of dichloromethane, then trifluoroacetic acid (25.03 mmol, 1.85 mL, 10 eq) was added dropwise thereto, and the reaction mixture was stirred for 2 hours at 24° C. The reaction mixture was added with saturated sodium bicarbonate to adjust the pH of the reaction mixture to about 7, and the reaction mixture was extracted with dichloromethane (50 mL*2). The organic phases were combined, washed with 20 mL of water, dried over anhydrous sodium sulfate, and then filtered, and the filtrate was concentrated under reduced pressure to obtain a crude product. The crude product was purified by column chromatography (dichloromethane:methanol=1:0 to 20:1) to obtain 007-2. LCMS: m/z=303.1 [M+1]⁺.

### Step 3: Synthesis of Compound 007-3

007-2 (0.1 g, 0.33 mmol, 1 eq) and B-1 (95.0 mg, 0.3 mmol, 0.9 eq) were dissolved in 1 mL of acetonitrile, then potassium carbonate (0.18 g, 1.34 mmol, 4 eq) was added thereto, and the reaction mixture was heated to 60° C. and stirred for 12 hours. Acetonitrile was removed by rotary evaporation under reduced pressure, then the reaction mixture was diluted with 10 mL of water and extracted with ethyl acetate (30 mL*2). The organic phases were combined, dried over anhydrous sodium sulfate, filtered, and the filtrate was concentrated under reduced pressure to obtain the crude product. The crude product was purified by column chromatography (dichloromethane:methanol=1:0 to 10:1) to obtain 007-3. LCMS: m/z=581.2 [M+1]⁺.

Step 4: Synthesis of compounds 007-3-P1 and 007-3-P2

007-3 was purified by preparative SFC (column model: DAICEL CHIRALPAK IC (250 mm*30 mm*10 m); mobile phase: phase A was supercritical carbon dioxide, and phase B was methanol; gradient (B): 5% to 50%) to obtain compound 007-3-P1 and compound 007-3-P2, which were subjected to chiral analysis SFC detection (column model: Chiralpak IG-3, 50×4.6 mm I.D., 3 m); mobile phase: phase A was supercritical carbon dioxide, and phase B was methanol; gradient (B %): 5% to 50%), showing that the retention time for compound 007-3-P1 was 0.932 minutes, with an ee value of 100%, and the retention time for compound 007-3-P2 was 1.206 minutes, with an ee value of 97.8%.

### Step 5: Synthesis of Compounds 007 and 007′

007-3-P1 (0.12 g, 0.21 mmol, 1 eq), methanol (0.5 mL), tetrahydrofuran (0.5 mL), water (0.5 mL), and lithium hydroxide monohydrate (26.2 mg, 0.62 mmol, 3 eq) were sequentially added to a dry reaction flask, and the reaction system was replaced with nitrogen and stirred for 3 hours at 28° C. The reaction mixture was concentrated under reduced pressure and then purified by preparative high performance liquid chromatography (method: column: Phenomenex C18 75*30 mm*3 m; mobile phase: [water (ammonium bicarbonate)-acetonitrile]; gradient (acetonitrile) %: 15% to 45%) to obtain 007. ¹H NMR (400 MHz, CDCl₃) δ ppm 8.80 (br s, 1H), 7.36-7.66 (m, 2H), 6.63-6.78 (m, 3H), 5.07 (br s, 1H), 4.18-4.60 (m, 4H), 3.92 (br s, 2H), 3.23 (br s, 1H), 2.74 (br s, 6H), 2.36 (br s, 2H), 2.07 (br s, 3H), 1.86 (br s, 2H). LCMS: m/z=553.2 [M+1]⁺.

Using compound 007-3-P2 as raw material, compound 007′ was synthesized by referring to the synthesis method of step 5 of Example 7. ¹H NMR (400 MHz, CDCl₃) δ ppm 8.82 (br s, 1H), 7.36-7.64 (m, 2H), 6.63-6.78 (m, 3H), 5.06 (br s, 1H), 4.18-4.58 (m, 4H), 3.93 (br s, 2H), 3.24 (br s, 1H), 2.74 (br s, 6H), 2.36 (br s, 2H), 2.05 (br s, 3H), 1.86 (br s, 2H). LCMS: m/z=553.2 [M+1]⁺.

### Example 8

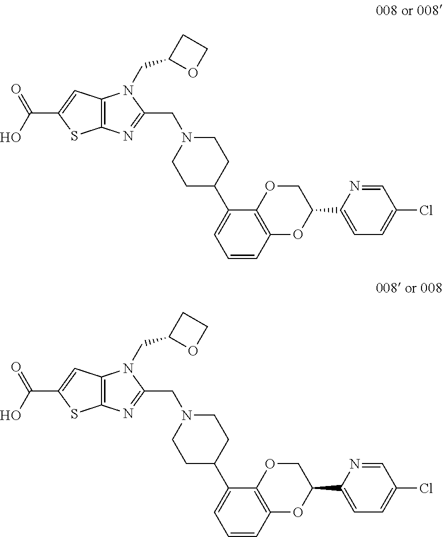 [SMILES: O=C(O)c1cc2c(nc(CN3CCC(c4cccc5c4OC[C@H](c4ccc(Cl)cn4)O5)CC3)n2C[C@@H]2CCO2)s1 ; O=C(O)c1cc2c(nc(CN3CCC(c4cccc5c4OC[C@@H](c4ccc(Cl)cn4)O5)CC3)n2C[C@@H]2CCO2)s1]

### Synthetic Route:

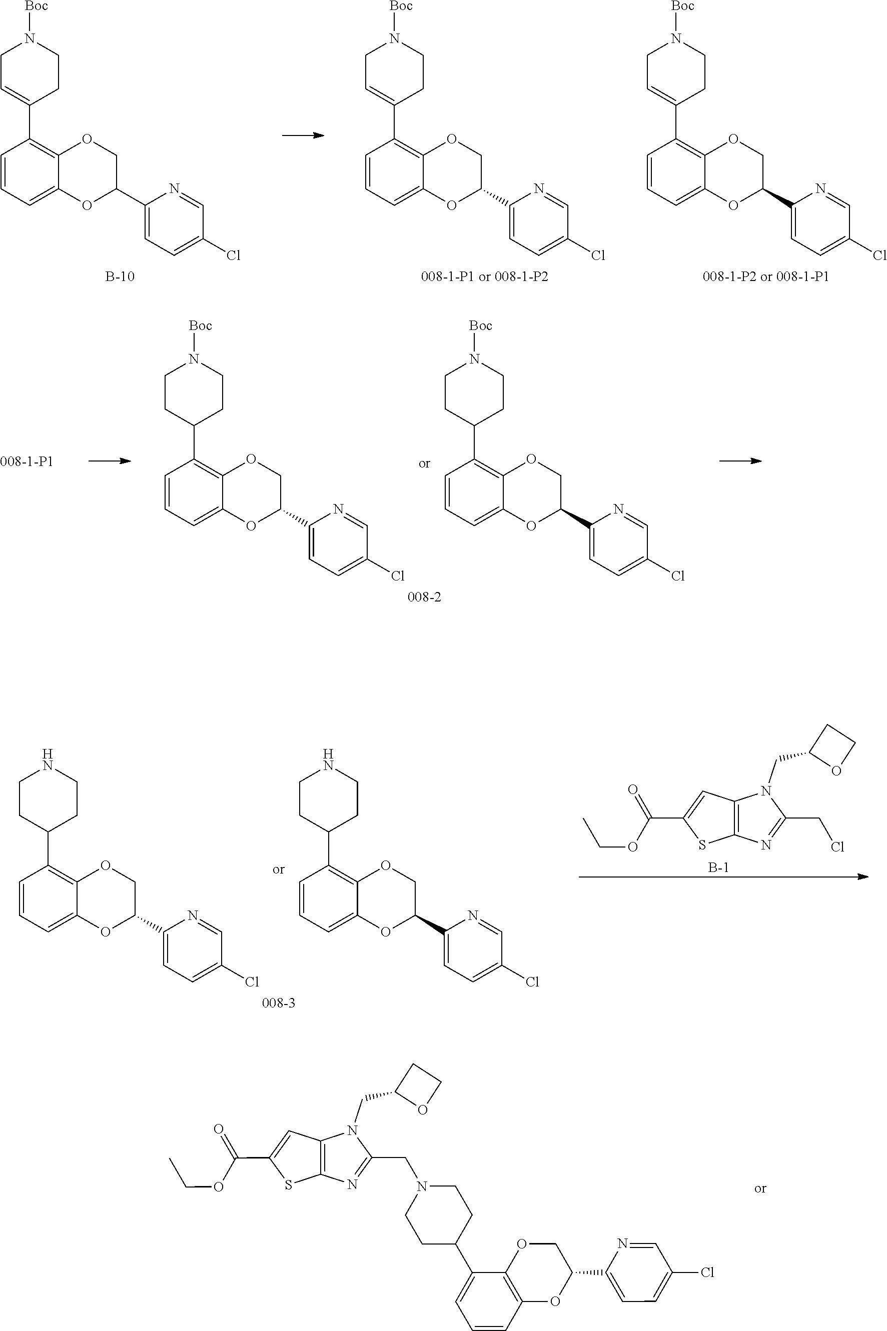

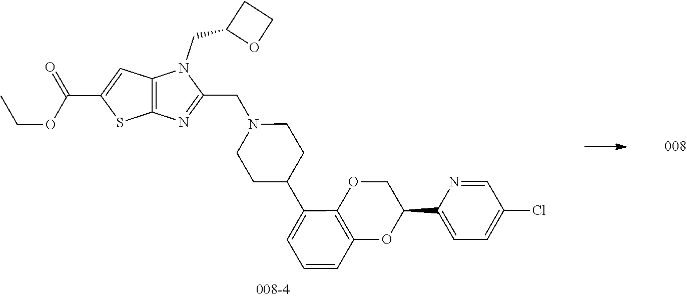 [SMILES: CCOC(=O)c1cc2c(nc(CN3CCC(c4cccc5c4OC[C@@H](c4ccc(Cl)cn4)O5)CC3)n2C[C@@H]2CCO2)s1]

### Step 1: Synthesis of 008-1-P1 and 008-1-P2

B-10 was subjected to chiral separation [separation method: chromatographic column: DAICEL CHIRALCEL OJ (250 mm*30 mm, 10 m); mobile phase: phase A was supercritical carbon dioxide, and phase B was methanol; gradient (B %): 30% to 30%] to obtain compound 008-1-P1 and compound 008-1-P2.

Compound 008-1-P1: Rt of 2.322 minutes, an ee value of 99.92%. Detection method: chromatographic column Chiralcel OJ-3, 150×4.6 mm I.D., 3 m; mobile phase: phase A was supercritical carbon dioxide, and phase B was methanol (0.1% isopropylamine); gradient (B %): 10% to 10%. ¹H NMR (400 MHz, CDCl₃) δ ppm 8.58 (s, 1H), 7.73 (dd, J=2.1, 8.4 Hz, 1H), 7.51 (d, J=8.4 Hz, 1H), 7.00-6.93 (m, 1H), 6.91-6.85 (m, 1H), 6.83-6.75 (m, 1H), 5.83 (br s, 1H), 5.27 (br d, J=5.9 Hz, 1H), 4.61 (td, J=1.2, 11.3 Hz, 1H), 4.17 (dd, J=7.5, 11.3 Hz, 1H), 4.05 (br s, 2H), 3.60 (br d, J=3.5 Hz, 2H), 2.50 (br s, 2H), 1.50 (s, 9H). LCMS: m/z=451.2 [M+Na]⁺.

Compound 008-1-P2: Rt of 2.642 minutes, an ee value of 98.58%. Detection method: chromatographic column Chiralcel OJ-3, 150×4.6 mm I.D., 3 m; mobile phase: phase A was supercritical carbon dioxide, and phase B was methanol (0.1% isopropylamine); gradient (B %): 10% to 10%. ¹H NMR (400 MHz, CDCl₃) δ 8.57 (d, J=2.1 Hz, 1H), 7.73 (dd, J=2.5, 8.4 Hz, 1H), 7.51 (d, J=8.5 Hz, 1H), 6.98-6.93 (m, 1H), 6.87 (t, J=7.8 Hz, 1H), 6.82-6.78 (m, 1H), 5.83 (br s, 1H), 5.27 (dd, J=2.5, 7.4 Hz, 1H), 4.61 (dd, J=2.6, 11.3 Hz, 1H), 4.17 (dd, J=7.5, 11.3 Hz, 1H), 4.05 (br s, 2H), 3.60 (br d, J=3.6 Hz, 2H), 2.50 (br s, 2H), 1.50 (s, 9H). LCMS: m/z=451.2 [M+Na]⁺.

### Step 2: Synthesis of 008-2

008-1-P1 (290 mg, 676.14 μmol, 1 eq), methanol (30 mL), and tris(triphenylphosphine)rhodium(I) chloride (62.56 mg, 67.61 μmol, 0.1 eq) were sequentially added to a dry reaction flask, and the reaction mixture was stirred for 24 hours under H₂ atmosphere (60° C., 50 psi). The reaction mixture was filtered with diatomite, and the filter cake was washed with methanol (50 mL). The filtrate was concentrated under reduced pressure. The crude product was purified by preparative thin-layer chromatography (petroleum ether:ethyl acetate=5:1) to obtain 008-2. ¹H NMR (400 MHz, CDCl₃) δ ppm 8.58 (d, J=2.0 Hz, 1H), 7.73 (dd, J=2.2, 8.3 Hz, 1H), 7.51 (d, J=8.3 Hz, 1H), 6.92-6.85 (m, 2H), 6.82-6.75 (m, 1H), 5.27 (dd, J=2.3, 7.2 Hz, 1H), 4.61 (dd, J=2.3, 11.2 Hz, 1H), 4.22-4.10 (m, 5H), 3.10-3.00 (m, 1H), 2.90-2.73 (m, 2H), 1.87-1.79 (m, 1H), 1.78-1.70 (m, 1H), 1.49 (s, 9H). LCMS: m/z=453.2 [M+Na]⁺.

### Step 3: Synthesis of 008-3

Compound 008-2 (200 mg, 464.12 μmol, 1 eq), DCM (5 mL), and TFA (5.86 mmol, 433.79 μL, 12.62 eq) were added to a dry reaction flask, and the reaction system was replaced with nitrogen and stirred for 1 hour at 20° C. The reaction mixture was concentrated under reduced pressure to obtain the crude trifluoroacetate salt 008-3, which was directly used in the next reaction step. LCMS: m/z=331.1 [M+H]⁺.

### Step 4: Synthesis of 008-4

B-1 (146.07 mg, 464.02 μmol, 1 eq), 008-3 (153.5 mg, the crude trifluoroacetate salt), acetonitrile (5 mL), and potassium carbonate (192.39 mg, 1.39 mmol, 3 eq) were sequentially added to a dry reaction flask, and the reaction mixture was stirred for 12 hours at 60° C. Additional potassium carbonate (192.39 mg, 1.39 mmol, 3 eq) was added thereto, and the reaction mixture was stirred for another 2 hours at 60° C. The reaction mixture was cooled to room temperature, then quenched with water (10 mL), and extracted with ethyl acetate (3*10 mL). After phase separation, the organic phase was collected, sequentially washed with saturated brine (3*10 mL), dried over anhydrous sodium sulfate, and concentrated under reduced pressure to obtain a crude product. The crude product was purified by preparative thin-layer chromatography (petroleum ether:ethyl acetate=1:1) to obtain 008-4. ¹H NMR (400 MHz, CDCl₃) δ ppm 8.57 (d, J=2.4 Hz, 1H), 7.78-7.69 (m, 2H), 7.54-7.48 (m, 1H), 6.96-6.76 (m, 2H), 5.32-5.13 (m, 2H), 4.70-4.52 (m, 4H), 4.45-4.33 (m, 3H), 4.24-4.08 (m, 2H), 3.87-3.77 (m, 2H), 3.07-2.88 (m, 3H), 2.81-2.68 (m, 1H), 2.59-2.40 (m, 1H), 2.36-2.18 (m, 2H), 1.94-1.63 (m, 4H), 1.39 (br t, J=7.0 Hz, 3H). LCMS: m/z=609.2 [M+H]⁺.

### Step 5: Synthesis of 008

008-4 (180 mg, 295.50 μmol, 1 eq), tetrahydrofuran (4 mL), methanol (4 mL), water (4 mL), and sodium hydroxide (130.02 mg, 3.25 mmol, 11 eq) were sequentially added to a dry reaction flask, and the reaction mixture was stirred for 4 hours at 20° C. The pH of the reaction mixture was adjusted to 2-3 with 2 N hydrochloric acid aqueous solution, and the reaction mixture was extracted with ethyl acetate (3*10 mL). After phase separation, the organic phase was collected, sequentially washed with saturated brine (3*10 mL), dried over anhydrous sodium sulfate, filtered, and the filtrate was concentrated under reduced pressure to obtain the crude product. The crude product was purified by preparative HPLC (chromatographic column: Phenomenex C18 80*40 mm*3 m; mobile phase: [water (ammonium bicarbonate)-acetonitrile]; B %: 20% to 40%, 8 minutes) to obtain compound 008. Rt of 1.215 minutes, an ee value of 97.1%. Detection method: chromatographic column: Chiralcel OJ-3, 50×4.6 mm I.D., 3 m; mobile phase: A: carbon dioxide, phase B: methanol (0.1% isopropylamine); gradient (B %): 5% to 50%. ¹H NMR (400 MHz, CDCl₃) δ ppm 8.57 (s, 1H), 7.72 (dd, J=1.6, 8.4 Hz, 1H), 7.50 (br d, J=8.3 Hz, 1H), 7.33 (br s, 1H), 6.93-6.77 (m, 3H), 5.26 (br d, J=5.1 Hz, 1H), 5.15-5.03 (m, 1H), 4.76-4.48 (m, 4H), 4.43-4.29 (m, 1H), 4.18 (br dd, J=7.3, 10.9 Hz, 2H), 4.07 (br d, J=12.8 Hz, 1H), 3.69-3.56 (m, 2H), 3.15-2.93 (m, 1H), 2.77-2.53 (m, 3H), 2.47-2.32 (m, 1H), 2.08-1.83 (m, 4H). LCMS: m/z=581.1 [M+H]⁺.

Using 008-1-P2 as raw material, compound 008′ was prepared by referring to steps 2 to 5 of Example 8. Rt of 1.750 minutes, an ee value of 100%. Detection method: chromatographic column: Chiralcel OJ-3, 50×4.6 mm I.D., 3 m; mobile phase: A: carbon dioxide, phase B: methanol (0.1% isopropylamine); gradient (B %): 5% to 50%. ¹H NMR (400 MHz, CDCl₃) δ 8.57 (d, J=1.8 Hz, 1H), 7.72 (dd, J=2.0, 8.4 Hz, 1H), 7.50 (d, J=8.4 Hz, 1H), 7.29 (s, 1H), 6.92-6.78 (m, 3H), 5.26 (br d, J=5.7 Hz, 1H), 5.12-5.02 (m, 1H), 4.77-4.65 (m, 1H), 4.65-4.49 (m, 3H), 4.43-4.32 (m, 1H), 4.28-4.05 (m, 3H), 3.79-3.55 (m, 2H), 3.12-2.97 (m, 1H), 2.74-2.55 (m, 3H), 2.47-2.30 (m, 1H), 2.12-1.85 (m, 4H). LCMS: m/z=581.1 [M+H]⁺.

### Example 9

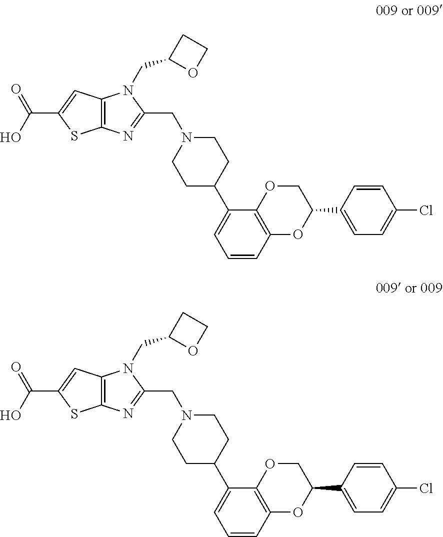 [SMILES: O=C(O)c1cc2c(nc(CN3CCC(c4cccc5c4OC[C@H](c4ccc(Cl)cc4)O5)CC3)n2C[C@@H]2CCO2)s1 ; O=C(O)c1cc2c(nc(CN3CCC(c4cccc5c4OC[C@@H](c4ccc(Cl)cc4)O5)CC3)n2C[C@@H]2CCO2)s1]

### Synthetic Route:

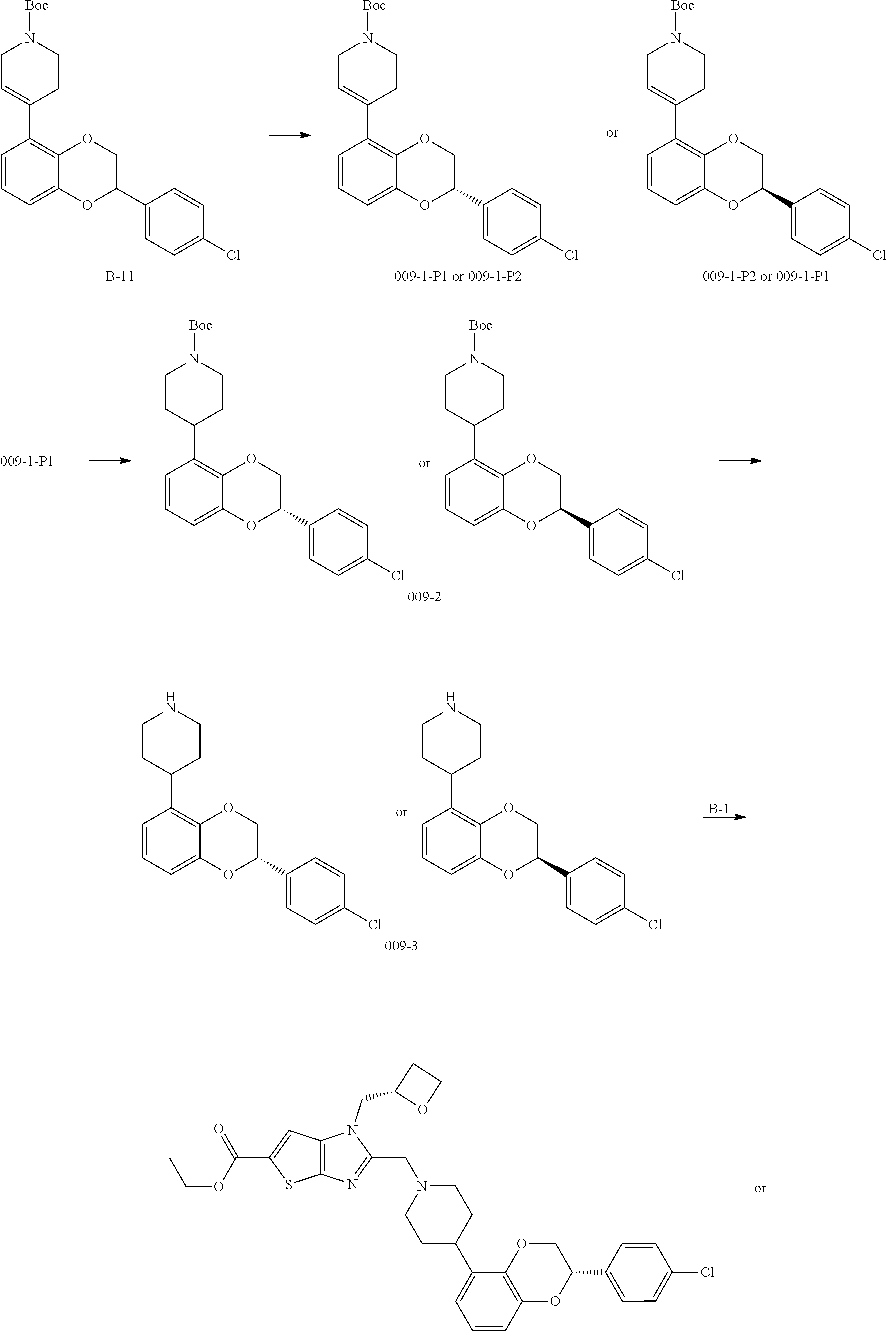

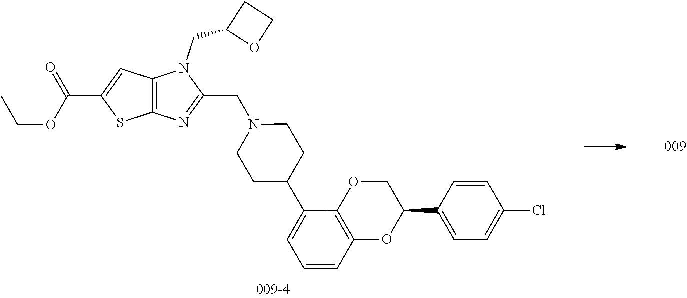 [SMILES: CCOC(=O)c1cc2c(nc(CN3CCC(c4cccc5c4OC[C@@H](c4ccc(Cl)cc4)O5)CC3)n2C[C@@H]2CCO2)s1]

### Step 1: Synthesis of 009-1-P1 and 009-1-P2

B-11 was separated by chiral HPLC (chromatographic column: DAICEL CHIRALCEL OJ (250 mm*30 mm, 10 m); mobile phase: phase A was supercritical carbon dioxide, and phase B was methanol, gradient (B %): 35% to 35%) to obtain 009-1-P1 and 009-1-P2.

Compound 009-1-P1 was detected by chiral analysis SFC (method: chromatographic column: Chiralcel OJ-3, 150×4.6 mm I.D., 3 m; mobile phase: A: supercritical carbon dioxide, B: methanol (0.1% isopropanol)), with a retention time of 3.094 minutes, an ee value of 100%. ¹H NMR (400 MHz, CDCl₃) δ ppm 7.45-7.34 (m, 4H), 6.95-6.90 (m, 1H), 6.86 (t, J=7.8 Hz, 1H), 6.81-6.77 (m, 1H), 5.84 (br s, 1H), 5.11 (dd, J=2.1, 8.7 Hz, 1H), 4.38 (dd, J=2.3, 11.4 Hz, 1H), 4.07 (br s, 2H), 3.97 (dd, J=8.9, 11.4 Hz, 1H), 3.62 (br s, 2H), 2.52 (br s, 2H), 1.50 (s, 9H). LCMS: m/z=450.2 [M+Na]⁺.

Compound 009-1-P2 was detected by chiral analysis SFC (method: chromatographic column: Chiralcel OJ-3, 150×4.6 mm I.D., 3 m; mobile phase: A: supercritical carbon dioxide, B: methanol (0.1% isopropanol), gradient (B %): 35% to 35%, retention time of 3.754 minutes, an ee value of 100%). ¹H NMR (400 MHz, CDCl₃) δ ppm 7.43-7.35 (m, 4H), 6.94-6.90 (m, 1H), 6.86 (t, J=7.8 Hz, 1H), 6.81-6.77 (m, 1H), 5.84 (br s, 1H), 5.11 (dd, J=2.2, 8.8 Hz, 1H), 4.38 (dd, J=2.4, 11.5 Hz, 1H), 4.06 (br s, 2H), 3.97 (dd, J=8.8, 11.4 Hz, 1H), 3.67-3.56 (m, 2H), 2.52 (br s, 2H), 1.50 (s, 9H). LCMS: m/z=450.2 [M+Na]⁺.

### Step 2: Synthesis of 009-2

009-1-P1 (260 mg, 607.59 μmol, 1 eq) and tris(triphenylphosphine)rhodium(I) chloride (56.22 mg, 60.76 μmol, 0.1 eq) were dissolved in methanol (26 mL), and the reaction mixture was stirred for 24 hours under H₂ atmosphere (60° C., 50 psi). The reaction mixture was filtered with diatomite. The filter cake was washed with dichloromethane (10 mL), and the resulting filtrate was concentrated under reduced pressure to obtain crude product. The crude product was purified by preparative thin-layer chromatography (petroleum ether:ethyl acetate=5:1) to obtain 009-2. ¹H NMR (400 MHz, CDCl₃) δ ppm 7.31 (q, J=8.5 Hz, 4H), 6.79 (d, J=4.5 Hz, 2H), 6.71 (br d, J=4.6 Hz, 1H), 5.03 (dd, J=1.9, 8.7 Hz, 1H), 4.36-4.26 (m, 1H), 4.16 (br d, J=1.6 Hz, 1H), 3.89 (dd, J=8.9, 11.3 Hz, 1H), 3.42 (s, 1H), 3.04-2.90 (m, 1H), 2.87-2.62 (m, 2H), 1.82-1.66 (m, 2H), 1.54 (dt, J=3.9, 12.8 Hz, 2H), 1.47-1.28 (m, 9H). LCMS: m/z=452.2 [M+Na]⁺.

### Step 3: Synthesis of 009-3

Compound 009-2 (0.22 g, 0.51 mmol, 1 eq), dichloromethane (2 mL), and trifluoroacetic acid (14.86 mmol, 1.1 mL, 29.03 eq) were added to a dry reaction flask, and the reaction system was replaced with nitrogen and stirred for 1 hour at 20° C. The reaction mixture was then concentrated under reduced pressure to obtain the crude trifluoroacetate salt 009-3, which was directly used in the next reaction step. LCMS: m/z=330.2 [M+1]⁺.

### Step 4: Synthesis of 009-4

B-1 (161.08 mg, 511.70 μmol, 1 eq), 009-3 (168.77 mg, the crude trifluoroacetate salt), and potassium carbonate (212.17 mg, 1.54 mmol, 3 eq) were dissolved in acetonitrile (5 mL), and the reaction mixture was stirred for 3 hours at 60° C. The reaction mixture was quenched with water (10 mL) and extracted with ethyl acetate (10 mL*3). After phase separation, the organic phase was collected, sequentially washed with saturated brine (10 mL), dried over anhydrous sodium sulfate, and the filtrate was concentrated under reduced pressure to obtain the crude product, which was purified by preparative thin-layer chromatography (dichloromethane:methanol=20:1) to obtain 009-4. ¹H NMR (400 MHz, CDCl₃) δ ppm 7.75 (s, 1H), 7.43-7.34 (m, 4H), 6.88-6.84 (m, 2H), 6.80 (br d, J=4.5 Hz, 1H), 5.25-5.16 (m, 1H), 5.12-5.07 (m, 1H), 4.70-4.61 (m, 1H), 4.56 (br d, J=3.5 Hz, 2H), 4.44-4.33 (m, 4H), 3.96 (dd, J=9.0, 11.4 Hz, 1H), 3.82 (br s, 1H), 3.02-2.92 (m, 2H), 2.80-2.70 (m, 1H), 2.50-2.41 (m, 1H), 2.34-2.21 (m, 2H), 1.71-1.62 (m, 6H), 1.40-1.35 (m, 3H). LCMS: m/z=608.2 [M+1]⁺.

### Step 5: Synthesis of 009

009-4 (200 mg, 328.87 μmol, 1 eq) was dissolved in THE (1 mL), MeOH (1 mL), and H₂O (1 mL), then lithium hydroxide monohydrate (41.40 mg, 986.61 μmol, 3 eq) was added thereto, and the reaction mixture was reacted at 20° C. for 36 hours. The pH of the reaction mixture was adjusted to 2-3 with 2 N hydrochloric acid aqueous solution, and the reaction mixture was extracted with ethyl acetate (3*5 mL). After phase separation, the organic phase was collected, sequentially washed with saturated brine (3*50 mL), dried over anhydrous sodium sulfate, and the filtrate was concentrated under reduced pressure to obtain the crude product. The crude product was purified by preparative HPLC (chromatographic column: Waters Xbridge BEH C18 100*30 mm*10 m; mobile phase: [water (ammonium bicarbonate)-acetonitrile]; B %: 40% to 60%) to obtain 009. Chiral analysis SFC detection (method: chromatographic column: Chiralcel OJ-3, 50×4.6 mm I.D., 3 m; mobile phase: A: carbon dioxide, B: MeOH (0.1% isopropanol), gradient (B %): 5% to 50%) showed an Rt of 1.373 minutes and an ee value of 100%. ¹H NMR (400 MHz, CDCl₃) δ ppm 7.41-7.35 (m, 4H), 7.32 (s, 1H), 6.86-6.82 (m, 3H), 5.09 (br d, J=7.8 Hz, 2H), 4.69 (br dd, J=4.8, 14.9 Hz, 1H), 4.59-4.52 (m, 2H), 4.38 (br d, J=10.1 Hz, 2H), 4.28-4.17 (m, 1H), 4.14-4.05 (m, 1H), 4.00-3.91 (m, 1H), 3.76-3.66 (m, 1H), 3.64-3.54 (m, 1H), 3.17-2.96 (m, 1H), 2.78-2.54 (m, 3H), 2.49-2.31 (m, 1H), 2.13-1.86 (m, 4H). LCMS: m/z=580.1 [M+1]⁺.

Using 009-1-P2 as raw material, compound 009′ was prepared by referring to steps 2 to 5 of Example 9. Rt of 1.914 minutes, an ee value of 100%. Detection method: chromatographic column: Chiralcel OJ-3, 50×4.6 mm I.D., 3 m; mobile phase: A: carbon dioxide, phase B: methanol (0.1% isopropylamine); gradient (B %): 5% to 50%. ¹H NMR (400 MHz, CDCl₃) δ ppm 7.38 (q, J=8.3 Hz, 4H), 7.32 (s, 1H), 6.89-6.79 (m, 3H), 5.09 (br d, J=7.4 Hz, 2H), 4.76-4.66 (m, 1H), 4.63-4.50 (m, 2H), 4.45-4.34 (m, 2H), 4.28-4.19 (m, 1H), 4.17-4.08 (m, 1H), 3.96 (dd, J=9.1, 11.0 Hz, 1H), 3.73-3.70 (m, 1H), 3.64-3.61 (m, 1H), 3.13-3.00 (m, 1H), 2.75-2.61 (m, 3H), 2.47-2.33 (m, 1H), 2.17-1.89 (m, 4H). LCMS: m/z=580.1 [M+1]⁺.

### Example 10

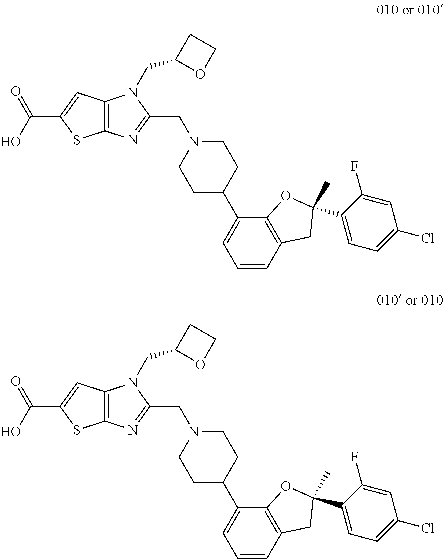 [SMILES: C[C@@]1(c2ccc(Cl)cc2F)Cc2cccc(C3CCN(Cc4nc5sc(C(=O)O)cc5n4C[C@@H]4CCO4)CC3)c2O1 ; C[C@]1(c2ccc(Cl)cc2F)Cc2cccc(C3CCN(Cc4nc5sc(C(=O)O)cc5n4C[C@@H]4CCO4)CC3)c2O1]

### Synthetic Route:

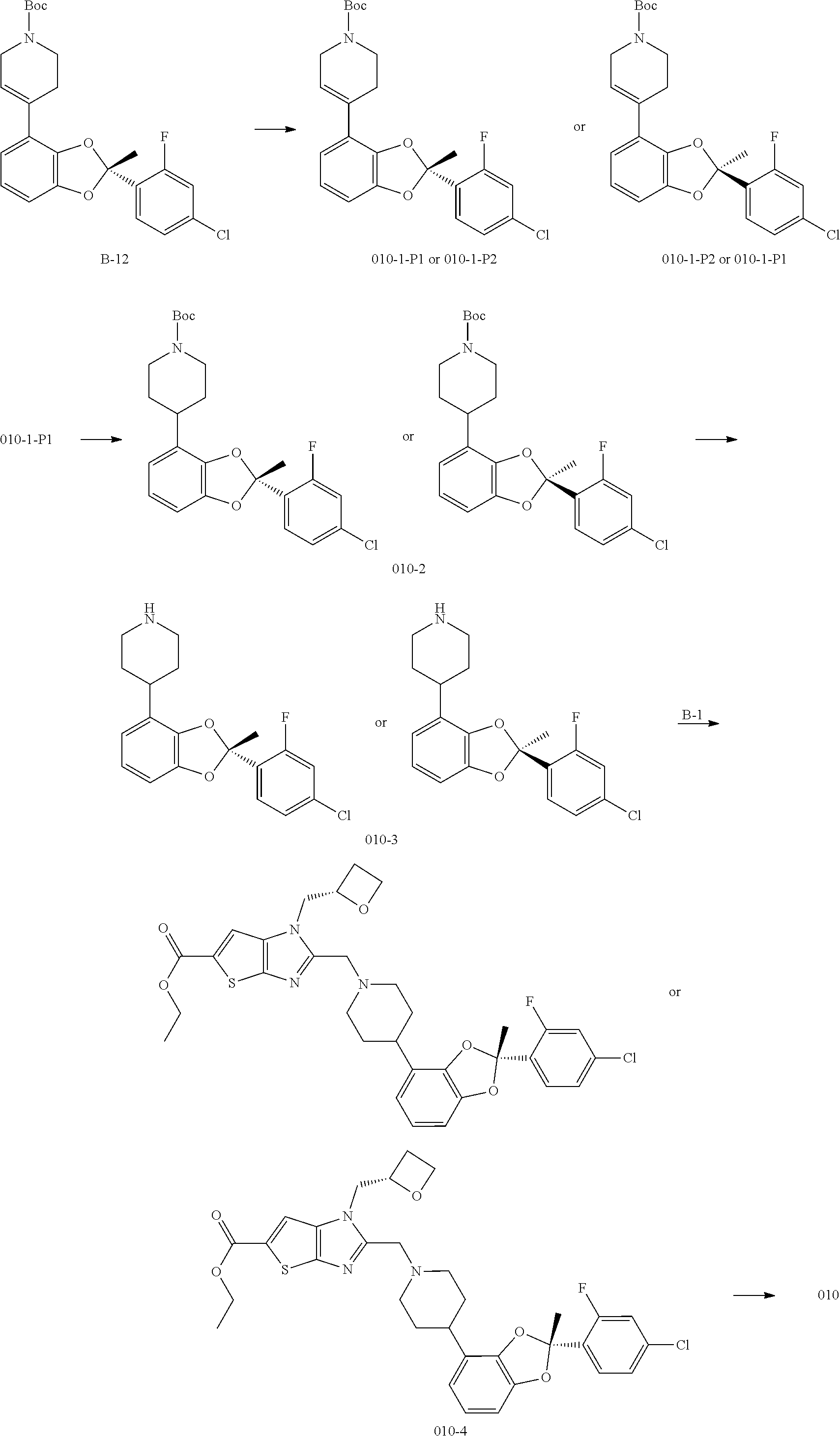

### Step 1: Synthesis of 010-1-P1 and 010-1-P2

B-12 was separated by chiral HPLC [chromatographic column: DAICEL CHIRALCEL OJ (250 mm*30 mm, 10 m); mobile phase: A: supercritical carbon dioxide, B: methanol; gradient (B %): 30% to 50%] to obtain compound 010-1-P1 and compound 010-1-P2.

010-1-P1 was detected by chiral analysis SFC (method: chromatographic column: Chiralcel OJ-3, 150×4.6 mm I.D., 3 m; phase A was supercritical carbon dioxide, phase B was methanol (0.1% isopropanol), gradient (B %): 30% to 50%), showing a retention time of 1.642 minutes and an ee value of 100%. LCMS: m/z=444.2 [M+1]⁺.

010-1-P2 was detected by chiral analysis SFC [method: chromatographic column: Chiralcel OJ-3, 150×4.6 mm I.D., 3 m; mobile phase A was supercritical carbon dioxide, B was methanol (0.1% isopropanol), gradient (B %): 30% to 50%], showing a retention time of 2.025 minutes and an ee value of 100%. LCMS: m/z=444.2 [M+1]⁺.

### Step 2: Synthesis of 010-2

010-1-P1 (550.00 mg, 1.24 mmol, 1 eq) and tris(triphenylphosphine)rhodium(I) chloride (114.63 mg, 123.89 μmol, 0.1 eq) were dissolved in methanol (10 mL), and the reaction mixture was stirred for 16 hours under H₂ atmosphere (60° C., 50 psi). The reaction mixture was filtered, the filter cake was washed with methanol (10 mL), and the resulting filtrate was concentrated under reduced pressure to obtain the crude product. The crude product was purified by column chromatography (petroleum ether:ethyl acetate=100:1 to 20:1) to obtain 010-2. ¹H NMR (400 MHz, CDCl₃) δ ppm 7.62-7.53 (m, 1H), 7.19-7.10 (m, 2H), 7.05-6.92 (m, 3H), 6.85 (d, J=7.5 Hz, 1H), 4.33-4.25 (m, 2H), 3.44 (br d, J=13.5 Hz, 2H), 2.99-2.90 (m, 2H), 1.88 (br d, J=11.9 Hz, 2H), 1.76 (br s, 2H), 1.55 (s, 3H), 1.50 (s, 9H). LCMS: m/z=468.1 [M+23]⁺.

### Step 3: Synthesis of 010-3

010-2 (90.00 mg, 201.82 μmol, 1 eq) was dissolved in dichloromethane (1 mL). The reaction system was replaced with nitrogen and cooled to 0° C., then trifluoroacetic acid (0.2 mL) was added thereto, and the reaction mixture was stirred for 1 hour at 20° C. The reaction mixture was directly concentrated under reduced pressure to obtain crude trifluoroacetate salt 010-3, which was used directly in the next reaction step without purification. LCMS: m/z=346.2 [M+1]⁺.

### Step 4: Synthesis of 010-4

010-3 (69.8 mg, crude trifluoroacetate salt) and B-1 (63.53 mg, 201.83 μmol, 1 eq) were dissolved in acetonitrile (1 mL), then potassium carbonate (111.58 mg, 807.32 μmol, 4 eq) was added thereto. The reaction system was replaced with nitrogen, and the reaction mixture was heated to 60° C. and stirred for 10 hours. The reaction mixture was diluted with water (5 mL) and extracted with ethyl acetate (5 mL*3). The resulting organic phase was washed with saturated brine (5 mL), dried over anhydrous sodium sulfate, filtered, and the filtrate was concentrated under reduced pressure to obtain the crude product. The crude product was purified by preparative thin-layer chromatography (PE:EA=1:1) to obtain 010-4.

¹H NMR (400 MHz, CDCl₃) δ ppm 7.84-7.69 (m, 1H), 7.61-7.47 (m, 1H), 7.17-7.09 (m, 2H), 7.01 (br s, 2H), 6.89-6.79 (m, 1H), 5.38-5.22 (m, 2H), 4.68-4.52 (m, 3H), 4.44-4.37 (m, 2H), 4.18-4.09 (m, 1H), 3.85 (br s, 2H), 3.54-3.41 (m, 2H), 3.12-2.89 (m, 2H), 2.88-2.56 (m, 2H), 2.49-2.29 (m, 2H), 1.74 (s, 3H), 1.56 (s, 3H), 1.44-1.38 (m, 3H), 1.29-1.27 (m, 1H). LCMS: m/z=624.2 [M+1]⁺.

### Step 5: Synthesis of 010

010-4 (70 mg, 112.15 μmol, 1 eq) and lithium hydroxide monohydrate (14.12 mg, 336.45 μmol, 3 eq) were dissolved in methanol (0.5 mL), tetrahydrofuran (0.5 mL), and water (0.5 mL), and the reaction system was replaced with nitrogen and stirred for 6 hours at 20° C. The reaction mixture was concentrated under reduced pressure and then purified by preparative HPLC (method: chromatographic column: Phenomenex C18 75*30 mm*3 m; mobile phase: [Water (ammonium bicarbonate)-acetonitrile]; gradient (acetonitrile) %: 25% to 55%) to obtain 010. Chiral analysis SFC detection (instrument: CAS-TJ-ANA-SFC—H (Waters UPCC with SQ Detector2; chromatographic column: Chiralpak AD-3, 50×4.6 mm I.D., 3 m; mobile phase: phase A was supercritical carbon dioxide, phase B was methanol (0.1% isopropanol), gradient: B % increased from 5% to 50% in 0.2 minutes and held for 2 minutes, and then decreased from 50% to 5% in 2.2 minutes) showed a retention time of 1.264 minutes and an ee value of 100%. ¹H NMR (400 MHz, CD₃OD) δ ppm 7.69-7.59 (m, 2H), 7.27-7.17 (m, 2H), 7.08-6.99 (m, 2H), 6.83 (s, 1H), 5.32-5.16 (m, 1H), 4.61 (s, 4H), 4.49-4.40 (m, 1H), 4.25 (s, 2H), 3.54-3.40 (m, 3H), 3.05-2.94 (m, 1H), 2.88-2.73 (m, 3H), 2.58-2.44 (m, 1H), 2.19-1.93 (m, 4H), 1.72 (s, 3H). LCMS: m/z=596.1 [M+1]⁺.

Using 010-1-P2 as raw material, compound 010′ was prepared by referring to steps 2 to 5 of Example 10. Rt of 2.155 minutes, an ee value of 100%. Detection method: chromatographic column: Chiralcel OJ-3, 50×4.6 mm I.D., 3 m; mobile phase: A: carbon dioxide, phase B: methanol (0.1% isopropylamine); gradient (B %): 5% to 50%. ¹H NMR (400 MHz, CDCl₃) δ ppm 7.67-7.60 (m, 2H), 7.26-7.17 (m, 2H), 7.06-6.99 (m, 2H), 6.85-6.79 (m, 1H), 5.26-5.17 (m, 1H), 4.72-4.54 (m, 3H), 4.41 (td, J=5.9, 9.1 Hz, 1H), 4.25 (br s, 2H), 3.55-3.34 (m, 4H), 3.03-2.92 (m, 1H), 2.87-2.71 (m, 3H), 2.54-2.43 (m, 1H), 2.22-1.96 (m, 4H), 1.72 (s, 3H). LCMS: m/z=596.1 [M+1]⁺.

### Example 11

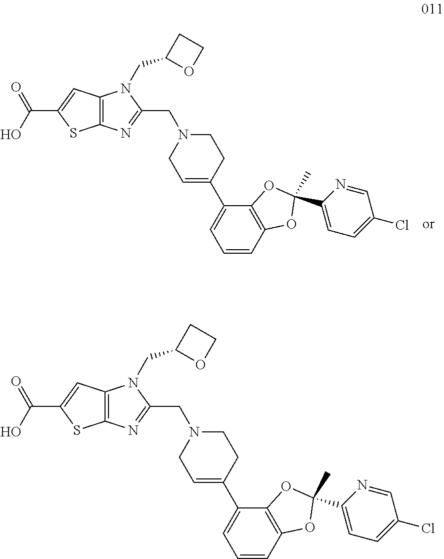 [SMILES: C[C@]1(c2ccc(Cl)cn2)Oc2cccc(C3=CCN(Cc4nc5sc(C(=O)O)cc5n4C[C@@H]4CCO4)CC3)c2O1 ; "or" ; C[C@@]1(c2ccc(Cl)cn2)Oc2cccc(C3=CCN(Cc4nc5sc(C(=O)O)cc5n4C[C@@H]4CCO4)CC3)c2O1]

### Synthetic Route:

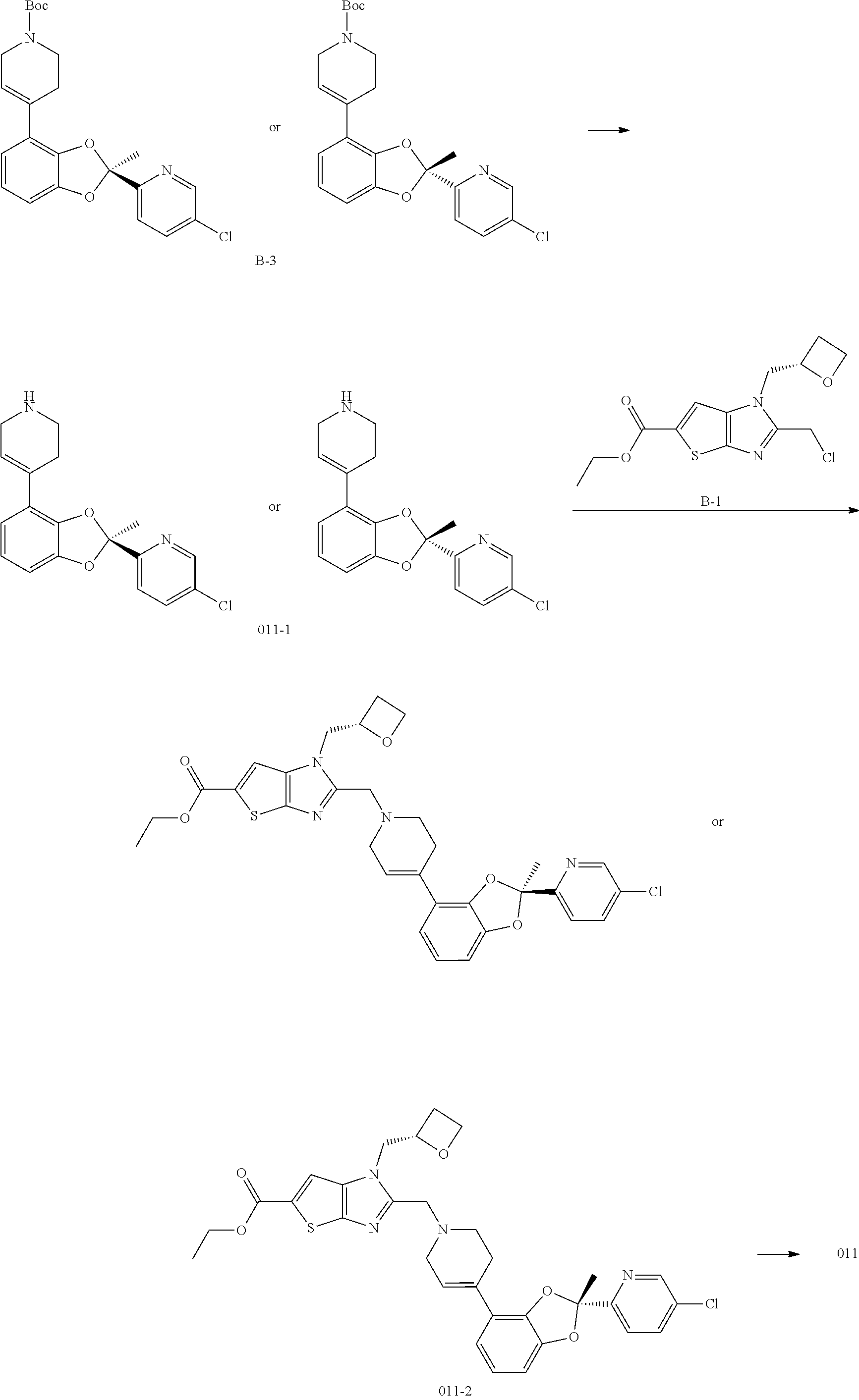

### Step 1: Synthesis of 011-1

B-3 (0.4 g, 0.93 mmol, 1 eq) was dissolved in DCM (5 mL). The reaction system was replaced with nitrogen and cooled to 0° C., then trifluoroacetic acid (1.0 mL) was added thereto, and the reaction mixture was stirred for 1 hour at 20° C. The reaction mixture was directly concentrated under reduced pressure to obtain crude trifluoroacetate salt 011-1, which was used directly in the next reaction step without purification. LCMS: m/z=329.1 [M+1]⁺.

### Step 2: Synthesis of 011-2

011-1 (0.24 g, crude trifluoroacetate salt) and B-1 (0.13 g, 0.40 mmol, 1 eq) were dissolved in acetonitrile (2 mL), then potassium carbonate (0.22 g, 0.16 mmol, 4 eq) was added thereto. The reaction system was replaced with nitrogen, and the reaction mixture was heated to 60° C. and stirred for 10 hours. The reaction mixture was diluted with water (10 mL) and extracted with ethyl acetate (3*15 mL). The resulting organic phase was washed with saturated brine (10 mL), dried over anhydrous sodium sulfate, filtered, and the resulting filtrate was concentrated under reduced pressure to obtain the crude product. The crude product was purified by preparative thin-layer chromatography (PE:EA=2:1) to obtain 011-2. LCMS: m/z=607.2 [M+1]⁺.

### Step 3: Synthesis of 011

011-2 (0.2 g, 0.33 mmol, 1 eq) was dissolved in methanol (1.5 mL), tetrahydrofuran (1.5 mL), and water (1.5 mL), and after complete dissolution, lithium hydroxide monohydrate (69.1 mg, 1.65 mmol, 5 eq) was added thereto, and the reaction mixture was stirred for 3 hours at 20° C. The reaction mixture was concentrated under reduced pressure, then 10 mL of ethyl acetate was added thereto, and the pH of the reaction mixture was adjusted to about 2 with 5 mL of 1 M hydrochloric acid. The resulting organic phase was extracted and concentrated under reduced pressure to obtain the crude product. The crude product was purified by p-HPLC (method: chromatographic column: Phenomenex C18 75*30 mm*3 m; mobile phase: [Water (ammonium bicarbonate)-acetonitrile]; gradient (acetonitrile) %: 25% to 55%)], and the resulting fraction was lyophilized to obtain 011. ¹H NMR (400 MHz, DMSO-d₆) δ ppm 8.71-8.73 (m, 1H), 7.95-8.02 (m, 1H), 7.70-7.55 (m, 2H), 6.91-6.78 (m, 3H), 6.34-6.30 (m, 1H), 5.07-4.97 (m, 1H), 5.60-4.40 (m, 4H), 4.35-4.30 (m, 1H), 3.83-3.78 (m, 1H), 3.75-3.68 (m, 1H), 2.72-2.60 (m, 4H), 2.33-2.25 (m, 3H), 2.01 (s, 3H). LCMS: m/z=579.1 [M+1]⁺.

### Example 12

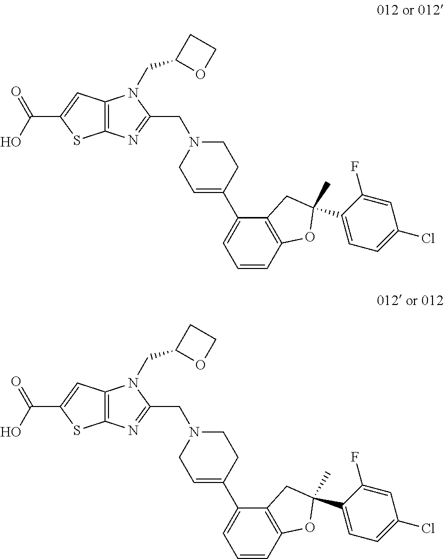 [SMILES: C[C@]1(c2ccc(Cl)cc2F)Cc2c(cccc2C2=CCN(Cc3nc4sc(C(=O)O)cc4n3C[C@@H]3CCO3)CC2)O1 ; C[C@@]1(c2ccc(Cl)cc2F)Cc2c(cccc2C2=CCN(Cc3nc4sc(C(=O)O)cc4n3C[C@@H]3CCO3)CC2)O1]

### Synthetic Route:

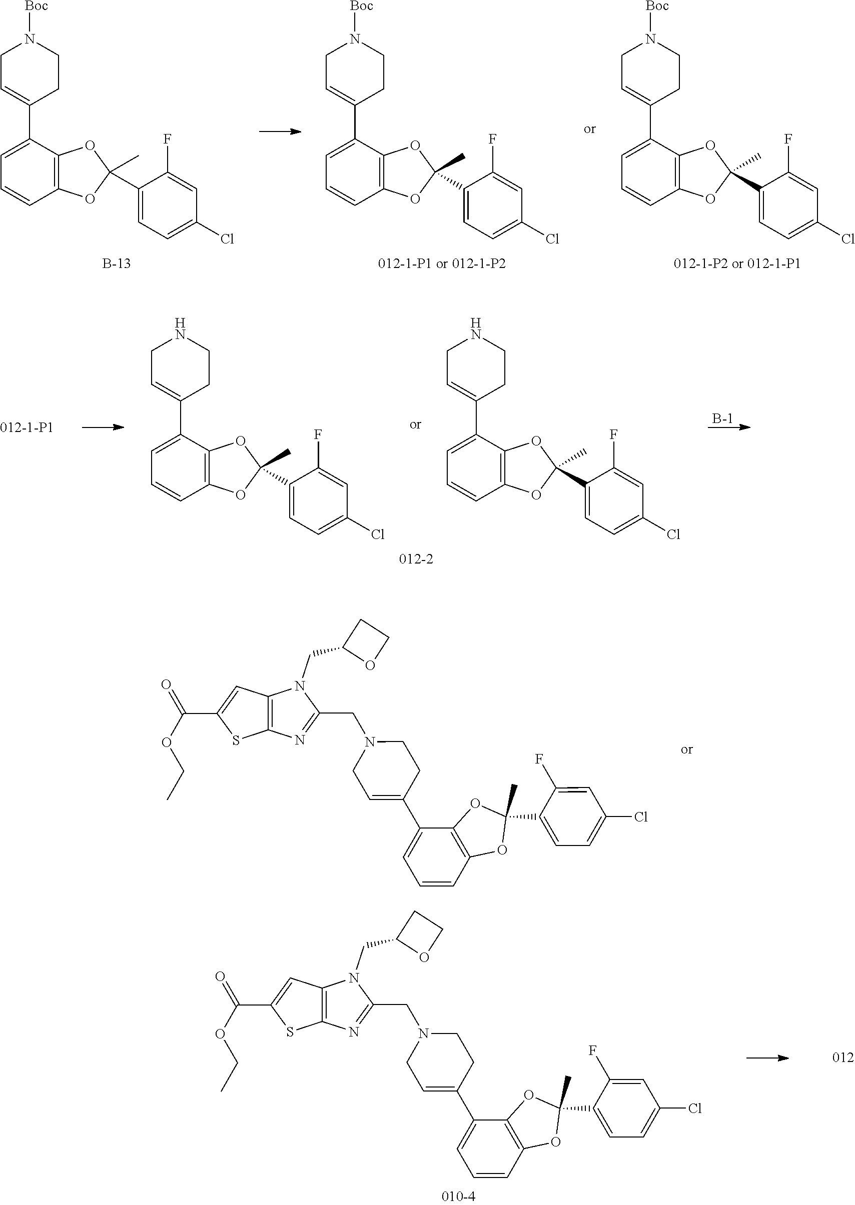

### Step 1: Synthesis of 012-1-P1 and 012-1-P2

B-13 was separated by chiral HPLC (chromatographic column: DAICEL CHIRALCEL OJ (250 mm*30 mm, 10 m); mobile phase: A: carbon dioxide, phase B: methanol; gradient (B %): 10% to 50%) to obtain 012-1-P1 and 012-1-P2.

012-1-P1 was detected by chiral analysis SFC (method: chromatographic column: ChiralpakAD-3, 150*4.6 mm I.D., 3 m; mobile phase: A: carbon dioxide, B: ethanol (0.1% isopropanol); gradient: (B %): 10% to 50%)), showing a retention time of 2.027 minutes and an ee value of 100%. LCMS: m/z=444.2 [M+1]⁺.

012-1-P2 was detected by chiral analysis SFC (method: chromatographic column: ChiralpakAD-3, 150*4.6 mm I.D., 3 m; mobile phase: A: carbon dioxide, B: methanol (0.1% isopropylamine); gradient: (B %): 10% to 50%)), showing a retention time of 2.221 minutes and an ee value of 100%. LCMS: m/z=444.2 [M+1]⁺.

### Step 2: Synthesis of 012-2

012-1-P1 (0.2 g, 450.51 μmol, 1 eq) was dissolved in dichloromethane (4 mL). The reaction system was replaced with nitrogen, then trifluoroacetic acid (5.40 mmol, 0.4 mL, 11.99 eq) was added thereto, and the reaction mixture was stirred for 3 hours at 20° C. 10 mL of saturated sodium carbonate and 5 mL of dichloromethane were added to the system, the pH of the aqueous phase was about 9, and the phases were separated. The organic phase was dried over anhydrous sodium sulfate, filtered, and the filtrate was concentrated under reduced pressure to obtain 012-2, which was used directly in the next reaction step without purification. LCMS: m/z=344.1 [M+1]⁺.

### Step 3: Synthesis of 012-3

012-2 (0.15 g, 433.73 μmol, 1 eq) and B-1 (136.53 mg, 433.73 μmol, 1 eq) were dissolved in acetonitrile (4 mL), then potassium carbonate (179.83 mg, 1.30 mmol, 3 eq) was added thereto. The reaction system was replaced with nitrogen, and the reaction mixture was heated to 60° C. and stirred for 2 hours. The system was filtered, the filter cake was washed with 10 mL of ethyl acetate, and the filtrate was concentrated under reduced pressure to obtain the crude product of 012-3. LCMS: m/z=622.2 [M+1]⁺.

### Step 4: Synthesis of 012

012-3 (0.15 g, 241.10 μmol, 1 eq) and lithium hydroxide monohydrate (50.58 mg, 1.21 mmol, 5 eq) were dissolved in methanol (3.0 mL), tetrahydrofuran (3.0 mL), and water (1.0 mL). The reaction system was replaced with nitrogen, and the reaction mixture was stirred for 6 hours at 20° C. The system was concentrated under reduced pressure to remove the solvent, then 10 mL of ethyl acetate was added thereto, and the pH was adjusted to about 3 with 1 M HCl. The phases were separated, the organic phase was concentrated under reduced pressure to obtain the crude product, which was purified by preparative HPLC (chromatographic column: Phenomenex Luna C18 75*30 mm*3 m; mobile phase: [water (formic acid)-acetonitrile]; gradient (acetonitrile) %: 45% to 85%), and then separated by preparative HPLC (chromatographic column: Waters Xbridge BEH C18 100*30 mm*10 m; mobile phase: [Water (ammonium bicarbonate)-acetonitrile]; gradient (acetonitrile) %: 30% to 60%) to obtain 012. Chiral analysis SFC detection (instrument: CAS-TJ-ANA-SFC—H (Waters UPCC with SQ Detector2; chromatographic column: Chiralpak AD-3, 50×4.6 mm I.D., 3 m; mobile phase: A: carbon dioxide, B: ethanol (0.1% isopropanol); gradient: the content of B increased from 5% to 50% in 0.2 minutes and held for 2 minutes, and then decreased from 50% to 5% in 2.2 minutes) showed a retention time of 1.142 minutes and an ee value of 100%. ¹H NMR (400 MHz, DMSO-d₄) δ ppm 7.76 (s, 1H), 7.55-7.43 (m, 2H), 7.27-7.23 (m, 1H), 7.21-7.13 (m, 1H), 6.82-6.76 (m, 2H), 5.82-5.76 (m, 1H), 5.02-4.97 (m, 1H), 4.67-4.59 (m, 1H), 4.47-4.35 (m, 2H), 4.31-4.25 (m, 1H), 3.81-3.72 (m, 2H), 3.14-3.08 (m, 2H), 2.62-2.56 (m, 4H), 2.45-2.32 (m, 4H), 1.70 (s, 3H). LCMS: m/z=594.2 [M+1]⁺.

Using 012-1-P2 as raw material, compound 012′ was prepared by referring to steps 2 to 4 of Example 12. Rt of 1.501 minutes, an ee value of 100%. Analytical method (chromatographic column: Chiralpak AD-3, 50×4.6 mm I.D., 3 m mobile phase: A: carbon dioxide, B: MeOH (0.1% isopropanol); gradient: (B %): 5% to 50%). ¹H NMR (400 MHz, CDCl₃) δ ppm 7.74 (s, 1H), 7.54-7.44 (m, 2H), 7.26-7.22 (m, 1H), 7.21-7.12 (m, 1H), 6.82-6.74 (m, 2H), 5.82-5.75 (m, 1H), 5.03-4.97 (m, 1H), 4.68-4.59 (m, 1H), 4.50-4.36 (m, 2H), 4.31-4.25 (m, 1H), 3.81-3.71 (m, 2H), 3.14-3.08 (m, 2H), 2.62-2.56 (m, 4H), 2.47-2.33 (m, 4H), 1.70 (s, 3H). LCMS: m/z=594.2 [M+1]⁺.

### Example 13

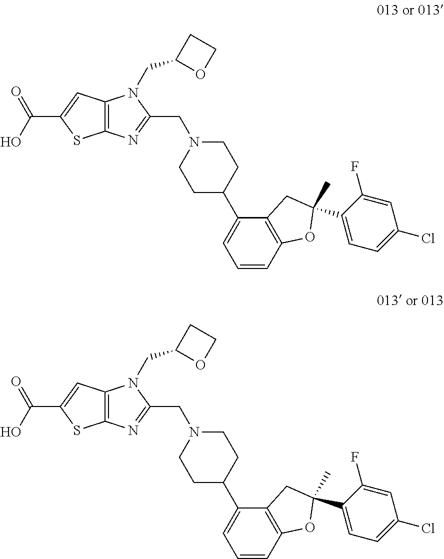 [SMILES: C[C@]1(c2ccc(Cl)cc2F)Cc2c(cccc2C2CCN(Cc3nc4sc(C(=O)O)cc4n3C[C@@H]3CCO3)CC2)O1 ; C[C@@]1(c2ccc(Cl)cc2F)Cc2c(cccc2C2CCN(Cc3nc4sc(C(=O)O)cc4n3C[C@@H]3CCO3)CC2)O1]

### Synthetic Route:

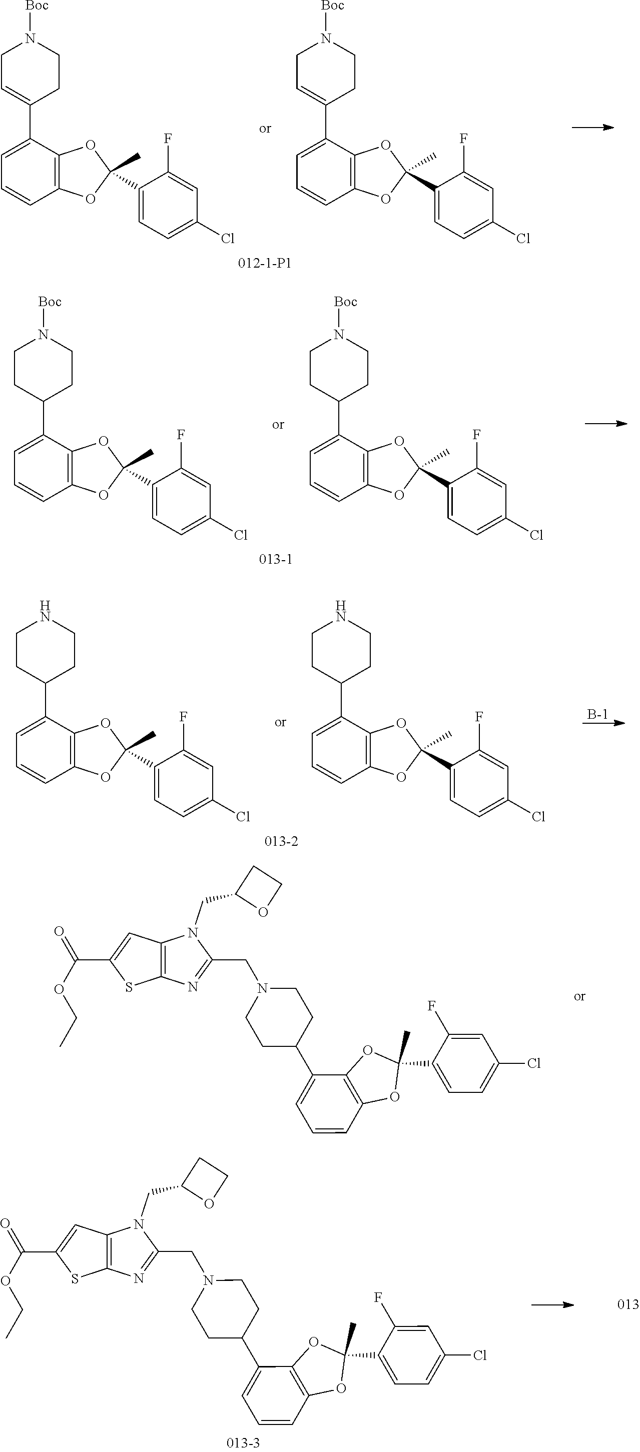

### Step 1: Synthesis of 013-1

012-1-P1 (0.2 g, 450.51 μmol, 1 eq) and tris(triphenylphosphine)rhodium(I) chloride (0.1 g, 108.08 μmol, 0.24 eq) were dissolved in methanol (10 mL), and the reaction mixture was stirred for 16 hours under H₂ atmosphere (50° C., 50 psi), and concentrated under reduced pressure to obtain the crude product of 013-1, which was used directly in the next reaction step. LCMS: m/z=390.0 [M−55]⁺.

### Step 2: Synthesis of 013-2

013-1 (387.90 mg, 869.82 μmol, 1 eq) was dissolved in DCM (4 mL), then TFA (5.40 mmol, 400.00 μL, 6.21 eq) was added thereto, and the system was stirred for 16 hours at 20° C. A saturated sodium carbonate aqueous solution was added to the reaction mixture, and the pH of the aqueous phase was adjusted to about 8. After phase separation, the organic phase was dried over anhydrous sodium sulfate, filtered, and the resulting filtrate was concentrated to obtain 013-2. LCMS: m/z=346.0 [M+1]⁺.

### Step 3: Synthesis of 013-3

013-2 (0.1 g, 289.15 μmol, 1 eq) and B-1 (110 mg, 349.44 μmol, 1.21 eq) were dissolved in acetonitrile (4 mL), then potassium carbonate (199.81 mg, 1.45 mmol, 5 eq) was added thereto. The reaction system was replaced with nitrogen, and the reaction mixture was heated to 60° C. and stirred for 2 hours. The reaction mixture was diluted with water (10 mL) and extracted with ethyl acetate (2*15 mL). The resulting organic phase was washed with saturated brine (10 mL), dried over anhydrous sodium sulfate, filtered, and the resulting filtrate was concentrated under reduced pressure to obtain the crude product of 013-3. LCMS: m/z=624.2 [M+1]⁺.

### Step 4: Synthesis of 013

013-3 (0.3 g, 480.64 μmol, 1 eq) was dissolved in MeOH (3 mL), H₂O (1 mL), and THE (3 mL), then lithium hydroxide monohydrate (100.85 mg, 2.40 mmol, 5 eq) was added thereto, and the system was stirred for 16 hours at 20° C. The system was concentrated under reduced pressure to remove the solvent, then 10 mL of ethyl acetate was added thereto, and the pH was adjusted to about 3 with 1 M HCl. The phases were separated, and the organic phase was concentrated under reduced pressure to obtain the crude product. The crude product was purified by preparative HPLC (chromatographic column: Phenomenex Luna C18 75*30 mm*3 m; mobile phase: [water (formic acid)-acetonitrile]; gradient (acetonitrile) %: 45% to 85%), and then purified by preparative HPLC (chromatographic column: Waters Xbridge BEH C18 100*30 mm*10 m; mobile phase: [Water (ammonium bicarbonate)-acetonitrile]; gradient (acetonitrile) %: 30% to 60%)] to obtain 013. ¹H NMR (400 MHz, DMSO-d₄) δ ppm 7.76 (s, 1H), 7.55-7.45 (m, 2H), 7.29-7.25 (m, 1H), 7.23-7.18 (m, 1H), 6.78-6.62 (m, 2H), 5.06-5.01 (m, 1H), 4.71-4.65 (m, 1H), 4.55-4.45 (m, 2H), 4.41-4.32 (m, 1H), 3.83-3.65 (m, 2H), 3.52-3.45 (m, 1H), 2.93-2.82 (m, 4H), 2.21-2.05 (m, 4H), 1.72-1.61 (m, 7H). LCMS: m/z=596.1 [M+1]⁺.

Using 012-1-P2 as raw material, compound 013′ was prepared by referring to steps 1 to 4 of Example 13. ¹H NMR (400 MHz, CDCl₃) δ ppm 7.74 (s, 1H), 7.53-7.44 (m, 2H), 7.29-7.25 (m, 1H), 7.23-7.18 (m, 1H), 6.78-6.62 (m, 2H), 5.06-5.01 (m, 1H), 4.71-4.65 (m, 1H), 4.55-4.45 (m, 2H), 4.41-4.32 (m, 1H), 3.82-3.62 (m, 2H), 3.52-3.45 (m, 1H), 2.93-2.82 (m, 4H), 2.21-2.05 (m, 4H), 1.72-1.61 (m, 7H). LCMS: m/z=596.1 [M+1]⁺.

### Experimental Example 1: In Vitro Cell Activity Assay

### 1. Materials

### 1) Cell Line

The host cell HEK293 for GLP-1 was constructed by WuXi AppTec (Shanghai) Co., Ltd.

### 2) Reagents

cAMP Detection Kit, Cisbio (Cat #62AM4PEJ); 1M HEPES, Invitrogen (Cat #15630-106); 1×HBSS, Invitrogen (Cat #14025); BSA, Sigma (Cat #B2064); IBMX, Sigma (Cat #I5879); Exenatide, Hao Yuan (HY-13443A).

### 3) Instruments

OptiPlate-384, White, PerkinElmer (Cat #6007290); 384 well plate for Echo, Labcyte (Cat #P-05525); EnVision, PerkinElmer; Vi-cell counter, Beckman (Cat #Vi-CELL™ XR Cell Viability Analyzer).

### 4) Compound Preparation

The compounds were prepared into a working solution with a concentration of 30 μM using DMSO. In this assay, each sample was used in an amount of 5 μL.

### 5) Experimental Buffers

24.5 mL of Hanks Buffer Saline Solution (HBSS), final concentration: lx;
125 μL of HEPES 1M, final concentration: 5 mM;
333 μL of 7.5% BSA Solution, final concentration: 0.1%;
25 μL of IBMX 500 mmol/L, final concentration: 0.5 mmol/L.

### 6) Preparation of Assay Reagents

Preparation of cAMP assay reagent: 250 μL of cAMP-D2 and 250 μL of anti-cAMP cryptate reagent were added to 4 mL of lysis buffer and mixed gently.

### 2. Experimental Methods

### a) Preparation of Compound Plate:

The compound to be tested was diluted 3-fold at 10 points with a starting concentration of 30 μM, and the dilution was completed using Bravo.

Exenatide, used as a control compound, was diluted 3-fold at 10 points with a starting concentration of 500 nM, and the dilution was completed using Bravo.

### b) Compound Transferring:

1) 100 nL of compound was transferred to an OptiPlate-384 plate using Echo.
2) The OptiPlate-384 plate was centrifuged at 1000 rpm for 5 seconds.

### c) Preparation of Cell Suspension

1) A GLP-1 cell cryopreservation tube was rapidly thawed in a 37° C. water bath.
2) The cell suspension was transferred to a 15 mL Transfer centrifuge tube and gently rinsed with 10 mL of HBSS.
3) The centrifuge tube was centrifuged at 1000 rpm for 1 minute at room temperature.
4) The supernatant was discarded.
5) The cells at the bottom were gently dispersed, then rinsed with 10 mL of HBSS, centrifuged to sedimentation, and finally resuspended in the experimental buffer.
6) The cell density and activity were measured using Vi-cell.
7) The concentration of GLP-1 cells was diluted to 2.0*105/mL with the experimental buffer.
8) 100 nL of the diluted cell suspension was transferred to the OptiPlate-384 plate.
9) The plate was incubated at room temperature for 30 minutes.

### d) The Assay Reagent was Added:

1) 10 μL of 800 nM gradient-diluted cAMP standard was added to the empty wells of the OptiPlate-384 plate.
2) 10 μL of cAMP assay reagent was added.
3) The OptiPlate-384 plate was covered with TopSeal-A film and incubated for 60 minutes at room temperature.

TopSeal-A was removed and the result was read in EnVision.

The experimental results are shown in Table 1:

TABLE 1
Assay results of cell activity in vitro

| | Human-GLP-1, |
|---|---|
| Compound | EC₅₀ (nM) |
| 001 | 0.54 |
| 002 | 0.88 |
| 003 | 0.51 |
| 006 | 4.81 |
| 008 | 2.90 |
| 009 | 1.49 |
| 011 | 1.20 |

Conclusion: The compounds of the present disclosure exhibit potent agonistic ability on GLP-1 receptors.

### Experimental Example 2: PK Parameter Testing In Vitro—Study on Time-Dependent Inhibition (TDI)

### Testing Purposes

The time-dependent inhibitory effect of the test compound on the activity of human liver microsomal cytochrome P450 isoenzyme CYP2C19 was determined.

### Experimental Method

The experiment was divided into two groups. In the first group, human liver microsomes (HLM) were used as the incubation system. The test articles with a series of concentrations were added to the incubation system, followed by the addition of a coenzyme factor (NADPH) solution. The preincubation was carried out at 37° C. for 30 minutes. After preincubation, a probe substrate solution was added thereto. After a certain period of incubation, the reaction was terminated. The amount of probe substrate metabolites generated in the incubation solution was determined, and the enzyme activity was calculated. In the second group, human liver microsomes (HLM) were used as the incubation system. The test articles with a series of concentrations were added to the incubation system, followed by the addition of potassium phosphate buffer. The preincubation was carried out at 37° C. for 30 minutes. After preincubation, a mixture of NADPH and the probe substrate was added thereto. After a certain period of incubation, the reaction was terminated. The amount of probe substrate metabolites generated in the incubation solution was determined, and the enzyme activity was calculated.

First, the test compound (10.0 mM) was diluted by gradient to prepare a working solution (100× the final concentration) with concentrations of 5.00, 1.65, 0.500, 0.165, 0.0500, 0.0165, and 0.00500 mM, respectively. Simultaneously, working solutions were respectively prepared for positive inhibitors of the P450 isoenzyme CYP2C19, probe substrate, and NADPH. Human liver microsomes, stored in a refrigerator at a temperature below −60° C., were thawed on ice, and once fully dissolved, were diluted with potassium phosphate buffer (PB) to prepare a working solution of a specific concentration (0.169 mg/mL).

Then, 147.5 μL of human liver microsomal working solution was added to the reaction plate, and the reaction plate was placed on ice for use; at this time, 2.50 μL of the test compound (N=1) and the working solution (N=2) of the positive control inhibitor at various concentrations were added to the corresponding wells, and the corresponding organic solvent was added to the group without inhibitors (test compound or positive inhibitor); the reaction plate was incubated for 10 minutes at 37° C., and then 50.0 μL of NADPH solution or potassium phosphate buffer was added to reaction wells of the first or second group, respectively, to start the reaction; the reaction plate was pre-incubated at 37° C. for 30 minutes; 50.0 μL of the substrate solution or a mixture of NADPH and the substrate was added to the reaction wells of the first or second group, respectively, to start the reaction. 20 minutes later, 250 μL of precooled acetonitrile solution (containing internal standards of 200 ng/mL tolbutamide and labetalol) was added to terminate the reaction; the reaction plate was placed on a shaker and shaken for 10 minutes to mix uniformly; then, the plate was centrifuged for 20 minutes at 4° C. and 4000 rpm; 200 μL of the supernatant was added to 200 μL of water to dilute the sample; the plate was finally sealed and shaken for 10 minutes to mix uniformly; the sample was then analyzed by LC/MS/MS.

Experimental results: as shown in Table 2.

TABLE 2
In vitro TDI test results

| CYP enzyme | | | |
|---|---|---|---|
| substrate | Compound | IC₅₀ (μM) | |
| CYP2C19 | 002 | (−)NADPH | >50 |
| | | (+)NADPH | 29.2 |
| | 003 | (−)NADPH | >50 |
| | | (+)NADPH | >50 |

Conclusion: The compounds of the present disclosure have a low risk of time-dependent inhibition of human liver microsomal cytochrome P450 isoenzyme 2C₁₉.

### Experimental Example 3: In Vitro PK Parameter Testing—HMS CLint (Liver) Test

Experimental purpose: to test the metabolic stability of the test sample in human and rat liver cells.

### Experimental Materials:

(1) test compound (10 mM), reference substance: 7-ethoxycoumarin (30 mM), 7-hydroxycoumarin (reference substance, 30 mM);
mouse hepatocytes with cell viability of 73.8%, supplier Cat No.: BioreclamationlVTM005052;
rat hepatocytes with cell viability of 78.3%, supplier Cat No.: BioreclamationlVTM00005-T;
canine hepatocytes with cell viability of 80.2%, supplier Cat No.: BioreclamationlVTM00205;
monkey liver cells with cell viability of 80.9%, supplier Cat No.: RILD HP—SXH-02M;
human hepatocytes with cell viability of 94.6%, supplier Cat No.: Bioreclamation IVTX008001.
(2) Buffer system:

Thawing medium: Williams' medium E containing 5% fetal bovine serum, 30% Percoll solution, and other auxiliary materials.

Incubation medium: Williams' medium E (without phenol red) containing 2 mM L-glutamine and 25 mM 4-(2-hydroxyethyl)-1-piperazineethanesulfonic acid.

Termination solution: acetonitrile containing 200 ng/mL tolbutamide and labetalol as internal standard.

Dilution solution: ultrapure water.

### Experimental Method:

1) An accurate amount of the positive control compound was dissolved in dimethyl sulfoxide (DMSO) to form a 30 mM solution
2) 10 mM of the test compound and 30 mM of the positive control compound were diluted into 1 mM and 3 mM, respectively, in a 96-well plate using DMSO.
3) 1 mM of the test compound and 3 mM of the positive control compound were diluted into 100 μM and 300 μM quantitative solutions using acetonitrile.
4) The frozen cells were thawed, separated, and suspended in culture medium, and then diluted to 0.5×10⁶ cells/mL using preheated culture medium.
5) 198 μL of preheated cell suspension was added to a 96-well plate.
6) 100 μL of termination solution (acetonitrile containing 200 ng/mL of tolbutamide and 200 ng/mL of labetalol as internal standards) was transferred in a set of pre-labeled 96-well plates.
7) 2 μL of 100 μM test compound or 300 μM positive control quantification solution was added, in duplicate, to each well of the 96-well plate.
8) TO samples were mixed for about 1 minute to achieve a uniform suspension, then 20 μL of each sample was immediately transferred into wells containing 100 μL of ice-cold termination solution, followed by mixing.
9) All plates were incubated at 37° C. in a 95% humidified incubator in 5% CO₂, and the reaction was started with a constant shaking at about 600 rpm.
10) At the time points of 15 minutes, 30 minutes, 60 minutes, and 90 minutes, the samples were mixed, and then at each time point, 20 μL of each sample was transferred to the wells containing 100 μL of ice-cold termination solution and mixed.
11) Medium control (MC) sample plates were prepared at TO and T90 (labeled TO-MC and T90-MC) by adding the same ingredients except the cell suspension to each well. A final concentration table was generated.
12) At each corresponding time point, the reaction was terminated by removing the plate from the incubator and mixing it with 100 μL of ice-cold termination solution.
13) The plate was immediately vortexed and shaken on a plate shaker for 10 minutes at 500 rpm. Then, all sample plates were centrifuged at 3220×g for 20 minutes at 4° C.
14) After centrifugation, the supernatant from the 35 μL/well sample plate was transferred to another set of pre-labeled 96-well plates, and 70 μL of ultrapure water was added to the 96-well plates according to the plate diagram.
15) The analytical plate was sealed and stored at 4° C. until LC-MS-MS analysis.

The residual rates of the test compound and the control compound were determined using the following formula:

$$Residental rate ( \% ) = \frac{peak area ratio of compound to internal standard at any time point}{peak area ratio of compound to internal standard at 0 min} \times 100 \%$$

The elimination rate constant (k) of the test compound and the control compound in hepatocytes was calculated by plotting the logarithm of the residual rate against time. The half-life (T_{1/2}) and the in vitro intrinsic clearance rate (CLᵢₙₜ) were obtained from the elimination rate constant k, using the following formulas:

$${T}_{1 / 2} = 0.693 / k ;$$

$${CL}_{int ( hep )} = k / cells per milliliter ( million cells / mL )$$

$${CL}_{int ( liver )} = {CL}_{int ( hep )} \times ratio of liver weight \text{} to body weight \times number of hepatocytes per gram of liver$$

The parameters of the various species in the formula are shown in Table 3:

TABLE 3
Parameters of various species

| | Ratio of | | |
|---|---|---|---|
| | liver | | Number of |
| | weight | | hepatocytes |
| | to body | Hepatic blood | (Number of |
| | weight | flow (Qₕ) | cells per |
| Species | (g/kg) | (mL/min/kg) | gram of liver) |
| Rat | 40 | 55.2 | 117 × 10⁶ |
| Dog | 32 | 30.9 | 215 × 10⁶ |
| Monkey | 30 | 43.6 | 120 × 10⁶ |
| Human | 20 | 20.7 | 139 × 10⁶ |

Experimental results: the experimental results are shown in Table 4.

TABLE 4
Results of HMS tests for different species

| | HMS (mL/min/kg) | | | |
|---|---|---|---|---|
| | | Cynomolgus | | |
| Compound | Human | monkeys | Dog | Rat |
| 002 | <17.8 | <23.0 | 115.4 | 138.7 |
| 003 | 19.4 | 41.6 | 58.7 | 5.1 |

Conclusion: The compounds of the present disclosure are slowly metabolized in hepatocytes of various species with little species variation.

### Experimental Example 4: In Vivo PK Parameter Testing

### Testing Purposes:

Male SD rats and male cynomolgus monkeys were used as test animals, and the plasma concentration of the compound was determined and the pharmacokinetic behavior was evaluated after a single oral administration.

### Experimental Method:

Two groups of healthy rats (fasted) were administered by intravenous injection (IV) and oral gavage (PO), each group with 2 rats. The vehicle was a mixture of 20% PEG400, 10% solutol, and 70% water. After mixing the compound to be tested with the vehicle, the mixture was vortexed and sonicated to prepare clear solutions of 0.2 mg/mL and 0.5 mg/mL. The intravenous injection dose for rats was 1.0 mg/kg, and the oral gavage dose was 5.0 mg/kg. After administration, the whole blood was collected at the time points of 0.083 hours, 0.25 hours, 0.5 hours, 1.0 hour, 2.0 hours, 4.0 hours, 8.0 hours, 12.0 hours, and 24.0 hours. Plasma was prepared from the whole blood and analyzed for drug concentration by LC-MS/MS. The pharmacokinetic parameters were calculated using Phoenix WinNonlin 6.3, and the results are shown in Table 5.

Two groups of healthy male cynomolgus monkeys (fasted) were administered by intravenous injection and oral gavage, each group with 2 monkeys. The vehicle was a 20% hydroxypropyl-β-cyclodextrin aqueous solution. The compound to be tested 002 was mixed with the vehicle, then vortexed, and sonicated to prepare clear solutions of 1.0 mg/mL and 2.0 mg/mL. The intravenous injection dose was 1.0 mg/kg, and the oral gavage dose was 2.0 mg/kg. The compound to be tested 003 was mixed with the vehicle, then vortexed, and sonicated to prepare clear solutions of 0.25 mg/mL and 10.0 mg/mL. The intravenous injection dose was 0.5 mg/kg, and the oral gavage dose was 10.0 mg/kg. After administration, the whole blood was collected at the time points of 0.083 hours, 0.25 hours, 0.5 hours, 1.0 hour, 2.0 hours, 4.0 hours, 8.0 hours, 12.0 hours, and 24.0 hours. Plasma was prepared from the whole blood and analyzed for drug concentration by LC-MS/MS. The pharmacokinetic parameters were calculated using Phoenix WinNonlin 6.3, and the results are shown in Table 6. Parameter meaning: Cₘₐₓ is the maximum plasma concentration; AUC is the exposure; T_{1/2} is the half-life; Vd is the apparent volume of distribution; Cl is the clearance rate; F % is the oral bioavailability.

TABLE 5
PK test results of compounds of the present disclosure in rats

| | AUC | T_{1/2} | | | |
|---|---|---|---|---|---|
| | (PO) | (PO) | Vd | Cl | |
| Compound | h*nmol/L | h | L/kg | mL/minutes/kg | F % |
| 002 | 3439 | 1.61 | 0.392 | 12.0 | 27.1 |

TABLE 6
PK test results of compounds of the present disclosure
in cynomolgus monkeys

| | AUC | T_{1/2} | | | |
|---|---|---|---|---|---|
| | (PO) | (PO) | Vd | Cl | |
| Compound | h*nmol/L | h | L/kg | mL/minutes/kg | F % |
| 002 | 42966 | 11.3 | 0.155 | 0.505 | 35.3 |
| 003 | 10716 | 8.52 | 0.489 | 2.85 | 23.6 |

Conclusion: The compound of the present disclosure has high oral exposure, long half-life, high bioavailability and good in vivo pharmacokinetic properties.

1. A compound of formula (P) or a pharmaceutically acceptable salt thereof, 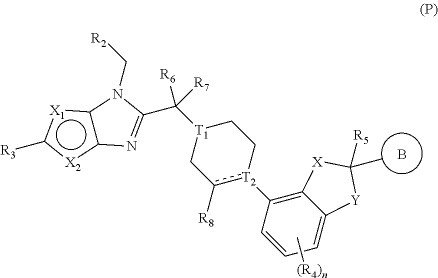
wherein [symbol]
is selected from a single bond and a double bond, and when T₂ is selected from N, [symbol] is selected from a single bond;
T₁ and T₂ are selected from N and CR₉;
X and Y are each independently selected from CH₂, O—CH₂, NH, O, and C(═O);
X₁ and X₂ are each independently selected from CH, N, O, and S, and the CH is optionally substituted by one F, Cl, Br, and CH₃;
ring B is selected from 5- to 6-membered heteroaryl, and the 5- to 6-membered heteroaryl is optionally substituted by 1, 2, or 3 R₁;
or, ring B is selected from phenyl, and the phenyl is optionally substituted by 1, 2, or 3 R₁, and in this case, X and Y are not simultaneously selected from O;
R₁ is selected from F, Cl, Br, I, OH, NH₂, CN, CH₃, and OCH₃;
R₂ is selected from 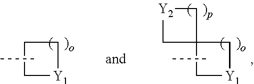
and the 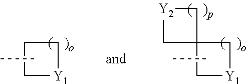
are optionally substituted by 1, 2, or 3 Rₐ;
Y₁ and Y₂ are each independently selected from CH₂, NH, and O;
o and p are each independently selected from 0, 1, 2, and 3;
R₃ is selected from —C(═O)—NH—R_{b}, —C(═O)—R_{b}, —C(═O)—NH—S(═O)₂—R_{b}, —S(═O)₂—NH—R_{b}, —S(═O)₂—R_{b}, —P(═O)(R_{b})₂, C₁₋₃ alkyl, tetrazolyl, isoxazolyl 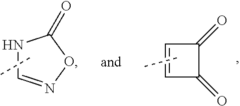
and the C₁₋₃ alkyl, tetrazolyl, isoxazolyl 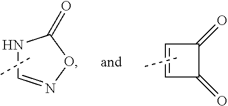
are optionally substituted by 1, 2, or 3 R_{b};
R₄ is selected from D, F, Cl, Br, I, and C₁₋₃ alkyl, and the C₁₋₃ alkyl is optionally substituted by 1, 2, or 3 R;
R₅ is selected from H, D, and CH₃, and the CH₃ is optionally substituted by 1, 2, or 3 R;
R₆, R₇, R₈, and R₉ are each independently selected from H, D, and C₁₋₃ alkyl, and the C₁₋₃ alkyl is optionally substituted by 1, 2, or 3 R;
n is selected from 0, 1, and 2;
each Rₐ is independently selected from F, Cl, Br, and I;
each R_{b} is independently selected from OH, CN, C₁₋₃ alkyl, C₁₋₃ alkoxy, C₁₋₃ alkylamino, and oxazolyl, and the C₁₋₃ alkyl, C₁₋₃ alkoxy, and oxazolyl are optionally substituted by 1, 2, or 3 R; and,
each R is independently selected from D, F, Cl, Br, and I.

2. The compound or the pharmaceutically acceptable salt thereof according to claim 1, wherein each R_{b} is independently selected from OH, CN, CH₃, CF₃, and OCH₃.

3. The compound or the pharmaceutically acceptable salt thereof according to claim 1, wherein R₂ is selected from 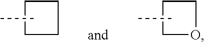
and the 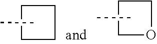
are optionally substituted by 1, 2, or 3 Rₐ.

4. The compound or the pharmaceutically acceptable salt thereof according to claim 1, wherein R₃ is selected from —COOH, —C(═O)—NH—CN, —C(═O)—NH—OH, —C(═O)—NH—OCH₃, —C(═O)—CF₃, —S(═O)₂—NH—CH₃, and —S(═O)₂—OH.

5. The compound or the pharmaceutically acceptable salt thereof according to claim 1, wherein R₄ is selected from F and CH₃.

6. The compound or the pharmaceutically acceptable salt thereof according to claim 1, wherein R₅ is selected from H, D, CH₃, CF₃, CHF₂, CHD₂, and CD₃.

7. The compound or the pharmaceutically acceptable salt thereof according to claim 1, wherein R₆, R₇, R₈, and R₉ are each independently selected from H, D, and CH₃, and the CH₃ is optionally substituted by 1, 2, or 3 R.

8. The compound or the pharmaceutically acceptable salt thereof according to claim 1, wherein ring B is selected from pyridyl, pyrimidinyl, pyrazinyl, pyridazinyl, pyrazolyl, imidazolyl, thiazolyl, and oxazolyl, and the pyridyl, pyrimidinyl, pyrazinyl, pyridazinyl, pyrazolyl, imidazolyl, thiazolyl, and oxazolyl are optionally substituted by 1, 2, or 3.

9. The compound or the pharmaceutically acceptable salt thereof according to claim 8, wherein ring B is selected from

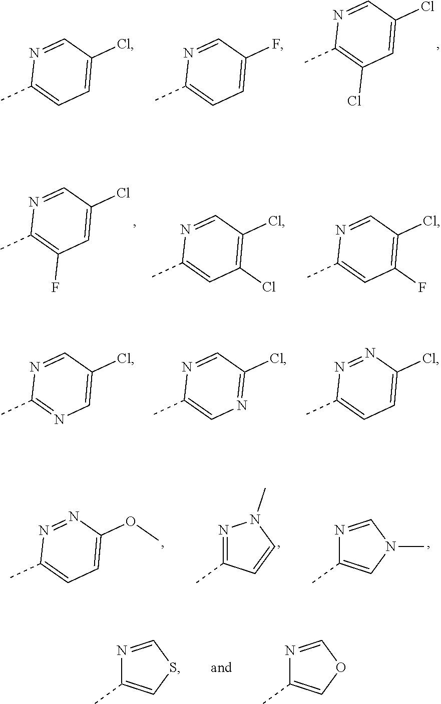

10. The compound or the pharmaceutically acceptable salt thereof according to claim 1, wherein the structural moiety 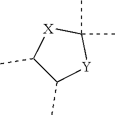
is selected from

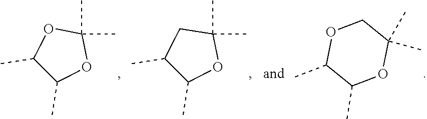

11. The compound or the pharmaceutically acceptable salt thereof according to claim 1, wherein ring B is phenyl, and the phenyl is optionally substituted by 1, 2, or 3 R₁, and the structural moiety 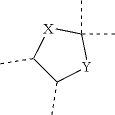
is selected from

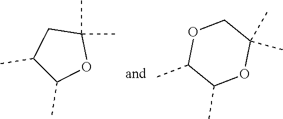

12. The compound or the pharmaceutically acceptable salt thereof according to claim 1, wherein the structural moiety 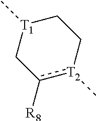
is selected from

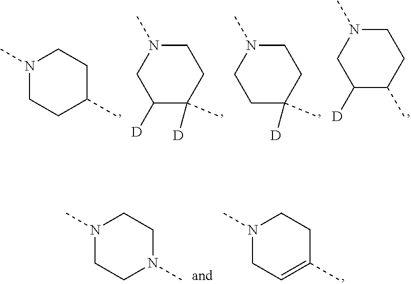

13. The compound or the pharmaceutically acceptable salt thereof according to claim 1, wherein the structural moiety 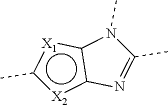
is selected from

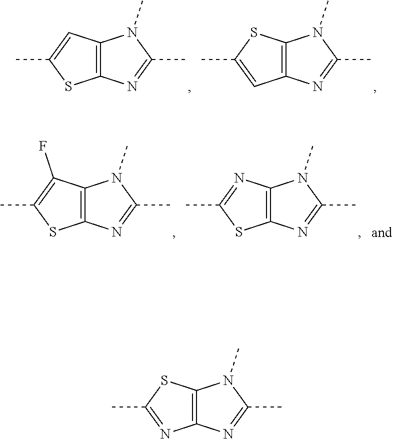

14. The compound or the pharmaceutically acceptable salt thereof according to claim 1, wherein the compound is selected from: 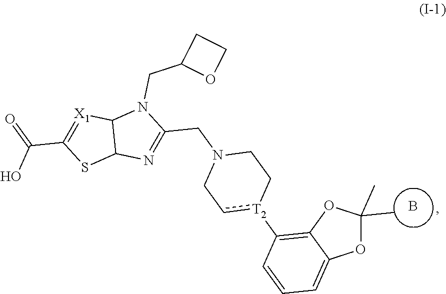
wherein [symbol]
is selected from a single bond and a double bond, and when T₂ is selected from N, [symbol] is selected from a single bond.

15. The compound or the pharmaceutically acceptable salt thereof according to claim 14, wherein the compound is selected from: 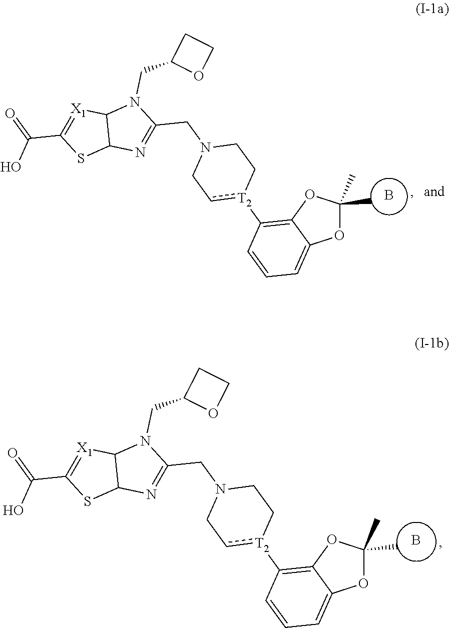
wherein [symbol] is selected from a single bond and a double bond, and when T₂ is selected from N, [symbol] is selected from a single bond.

16. A compound of the following formula or a pharmaceutically acceptable salt thereof, selected from,

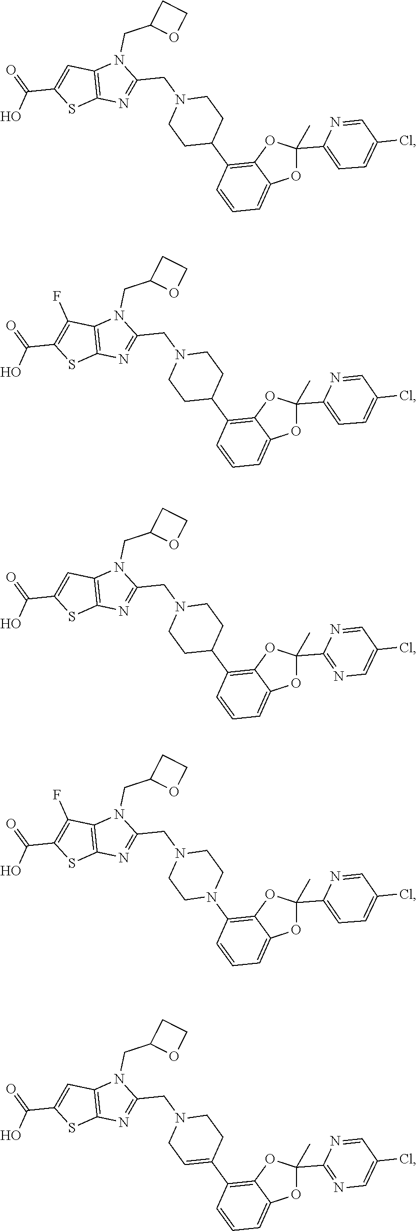 [SMILES: CC1(c2ccc(Cl)cn2)Oc2cccc(C3CCN(Cc4nc5sc(C(=O)O)cc5n4CC4CCO4)CC3)c2O1 ; CC1(c2ccc(Cl)cn2)Oc2cccc(C3CCN(Cc4nc5sc(C(=O)O)c(F)c5n4CC4CCO4)CC3)c2O1 ; CC1(c2ncc(Cl)cn2)Oc2cccc(C3CCN(Cc4nc5sc(C(=O)O)cc5n4CC4CCO4)CC3)c2O1 ; CC1(c2ccc(Cl)cn2)Oc2cccc(N3CCN(Cc4nc5sc(C(=O)O)c(F)c5n4CC4CCO4)CC3)c2O1 ; CC1(c2ncc(Cl)cn2)Oc2cccc(C3=CCN(Cc4nc5sc(C(=O)O)cc5n4CC4CCO4)CC3)c2O1]

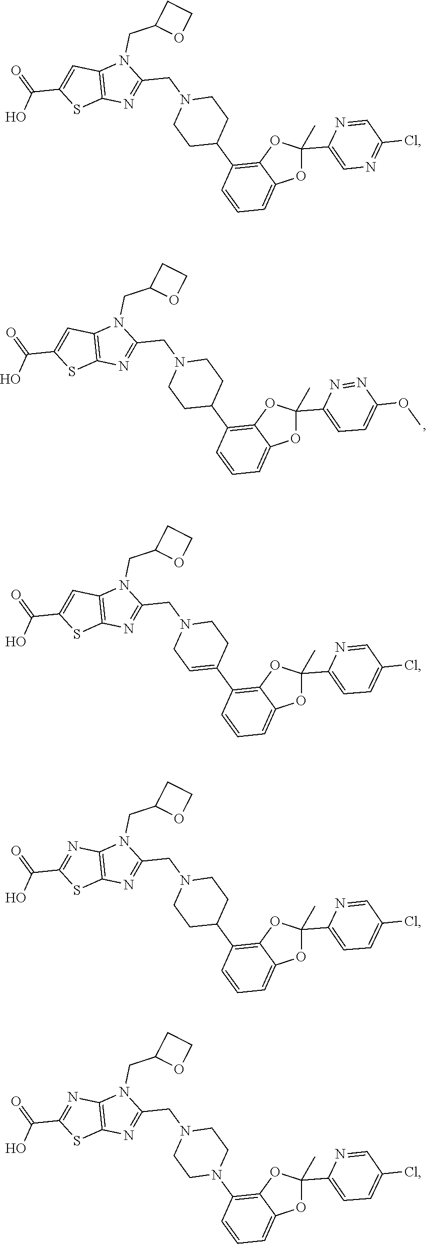 [SMILES: CC1(c2cnc(Cl)cn2)Oc2cccc(C3CCN(Cc4nc5sc(C(=O)O)cc5n4CC4CCO4)CC3)c2O1 ; COc1ccc(C2(C)Oc3cccc(C4CCN(Cc5nc6sc(C(=O)O)cc6n5CC5CCO5)CC4)c3O2)nn1 ; CC1(c2ccc(Cl)cn2)Oc2cccc(C3=CCN(Cc4nc5sc(C(=O)O)cc5n4CC4CCO4)CC3)c2O1 ; CC1(c2ccc(Cl)cn2)Oc2cccc(C3CCN(Cc4nc5sc(C(=O)O)nc5n4CC4CCO4)CC3)c2O1 ; CC1(c2ccc(Cl)cn2)Oc2cccc(N3CCN(Cc4nc5sc(C(=O)O)nc5n4CC4CCO4)CC3)c2O1]

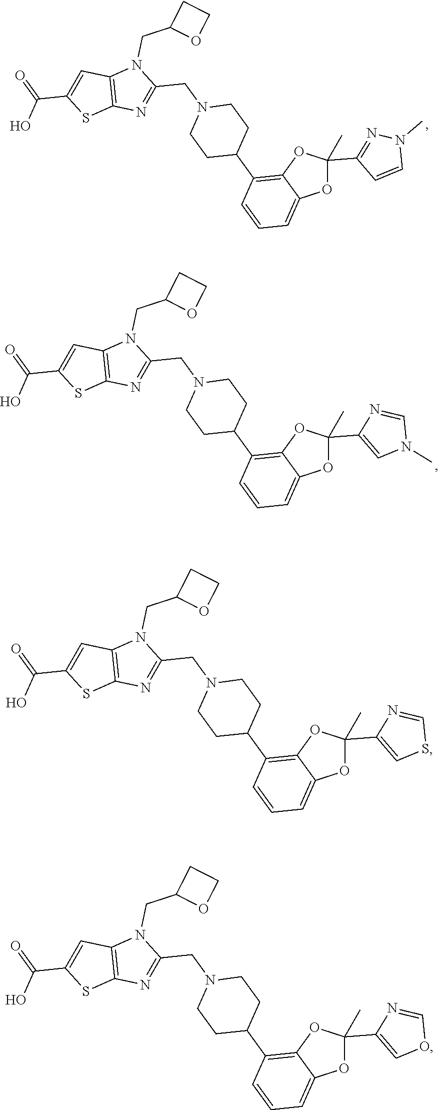 [SMILES: Cn1ccc(C2(C)Oc3cccc(C4CCN(Cc5nc6sc(C(=O)O)cc6n5CC5CCO5)CC4)c3O2)n1 ; Cn1cnc(C2(C)Oc3cccc(C4CCN(Cc5nc6sc(C(=O)O)cc6n5CC5CCO5)CC4)c3O2)c1 ; CC1(c2cscn2)Oc2cccc(C3CCN(Cc4nc5sc(C(=O)O)cc5n4CC4CCO4)CC3)c2O1 ; CC1(c2cocn2)Oc2cccc(C3CCN(Cc4nc5sc(C(=O)O)cc5n4CC4CCO4)CC3)c2O1]

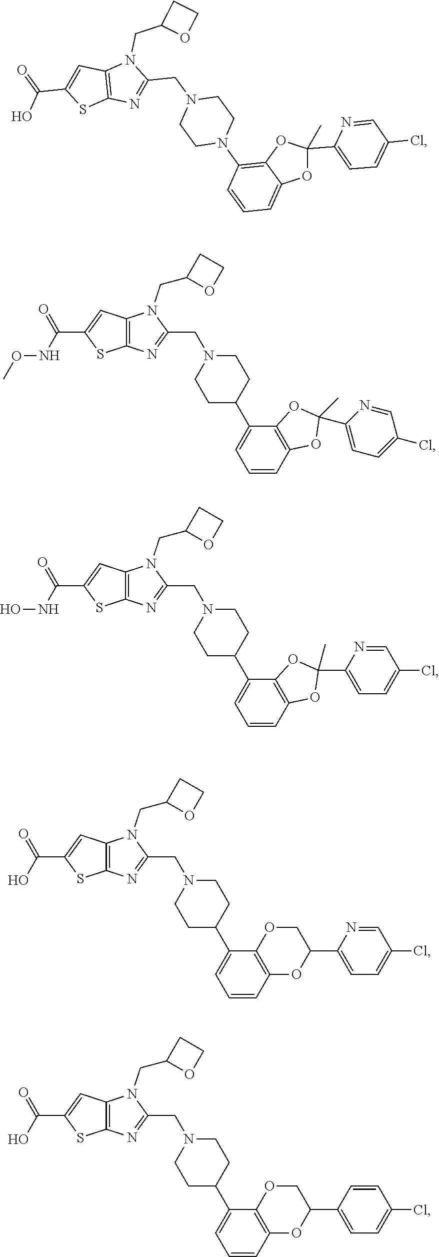 [SMILES: CC1(c2ccc(Cl)cn2)Oc2cccc(N3CCN(Cc4nc5sc(C(=O)O)cc5n4CC4CCO4)CC3)c2O1 ; CONC(=O)c1cc2c(nc(CN3CCC(c4cccc5c4OC(C)(c4ccc(Cl)cn4)O5)CC3)n2CC2CCO2)s1 ; CC1(c2ccc(Cl)cn2)Oc2cccc(C3CCN(Cc4nc5sc(C(=O)NO)cc5n4CC4CCO4)CC3)c2O1 ; O=C(O)c1cc2c(nc(CN3CCC(c4cccc5c4OCC(c4ccc(Cl)cn4)O5)CC3)n2CC2CCO2)s1 ; O=C(O)c1cc2c(nc(CN3CCC(c4cccc5c4OCC(c4ccc(Cl)cc4)O5)CC3)n2CC2CCO2)s1]

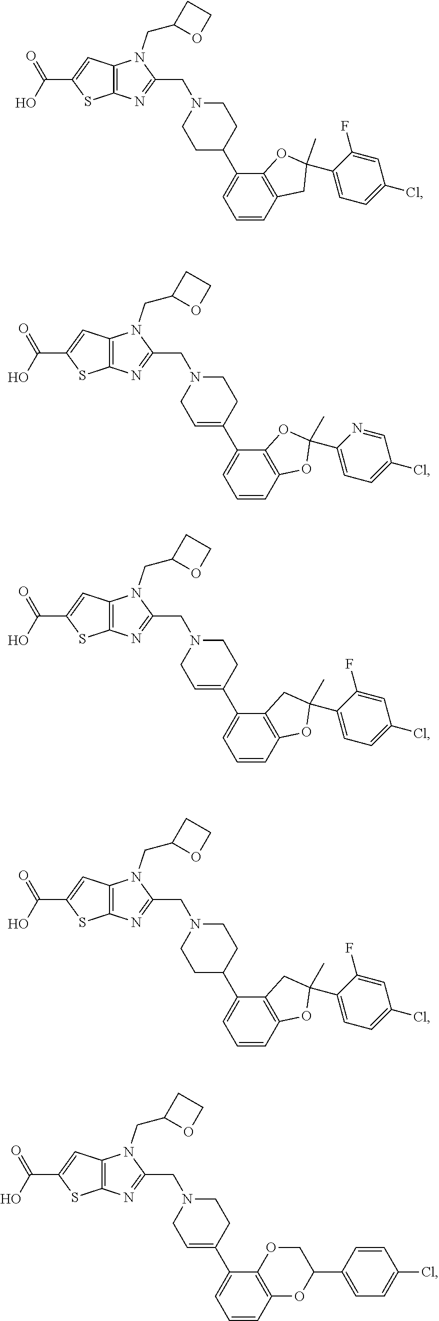 [SMILES: CC1(c2ccc(Cl)cc2F)Cc2cccc(C3CCN(Cc4nc5sc(C(=O)O)cc5n4CC4CCO4)CC3)c2O1 ; CC1(c2ccc(Cl)cn2)Oc2cccc(C3=CCN(Cc4nc5sc(C(=O)O)cc5n4CC4CCO4)CC3)c2O1 ; CC1(c2ccc(Cl)cc2F)Cc2c(cccc2C2=CCN(Cc3nc4sc(C(=O)O)cc4n3CC3CCO3)CC2)O1 ; CC1(c2ccc(Cl)cc2F)Cc2c(cccc2C2CCN(Cc3nc4sc(C(=O)O)cc4n3CC3CCO3)CC2)O1 ; O=C(O)c1cc2c(nc(CN3CC=C(c4cccc5c4OCC(c4ccc(Cl)cc4)O5)CC3)n2CC2CCO2)s1]

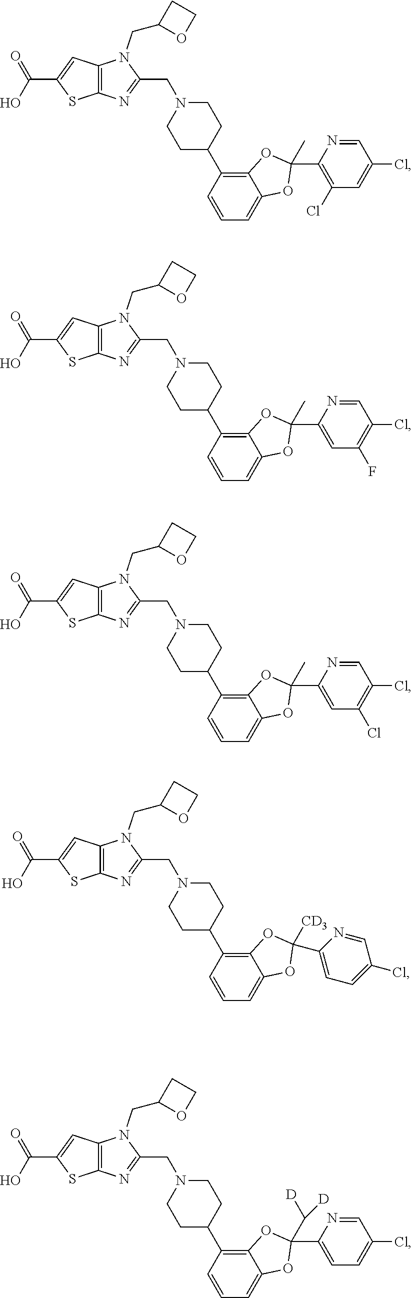 [SMILES: CC1(c2ncc(Cl)cc2Cl)Oc2cccc(C3CCN(Cc4nc5sc(C(=O)O)cc5n4CC4CCO4)CC3)c2O1 ; CC1(c2cc(F)c(Cl)cn2)Oc2cccc(C3CCN(Cc4nc5sc(C(=O)O)cc5n4CC4CCO4)CC3)c2O1 ; CC1(c2cc(Cl)c(Cl)cn2)Oc2cccc(C3CCN(Cc4nc5sc(C(=O)O)cc5n4CC4CCO4)CC3)c2O1 ; [2H]C([2H])([2H])C1(c2ccc(Cl)cn2)Oc2cccc(C3CCN(Cc4nc5sc(C(=O)O)cc5n4CC4CCO4)CC3)c2O1 ; [2H]C([2H])C1(c2ccc(Cl)cn2)Oc2cccc(C3CCN(Cc4nc5sc(C(=O)O)cc5n4CC4CCO4)CC3)c2O1]

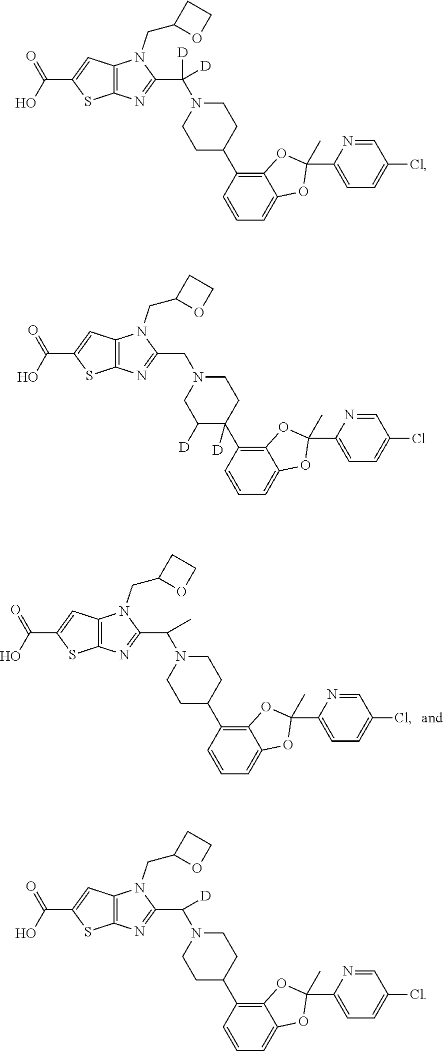 [SMILES: [2H]C([2H])(c1nc2sc(C(=O)O)cc2n1CC1CCO1)N1CCC(c2cccc3c2OC(C)(c2ccc(Cl)cn2)O3)CC1 ; [2H]C1CN(Cc2nc3sc(C(=O)O)cc3n2CC2CCO2)CCC1([2H])c1cccc2c1OC(C)(c1ccc(Cl)cn1)O2 ; CC(c1nc2sc(C(=O)O)cc2n1CC1CCO1)N1CCC(c2cccc3c2OC(C)(c2ccc(Cl)cn2)O3)CC1 ; [2H]C(c1nc2sc(C(=O)O)cc2n1CC1CCO1)N1CCC(c2cccc3c2OC(C)(c2ccc(Cl)cn2)O3)CC1]

17. The compound or the pharmaceutically acceptable salt thereof according to claim 16, selected from,

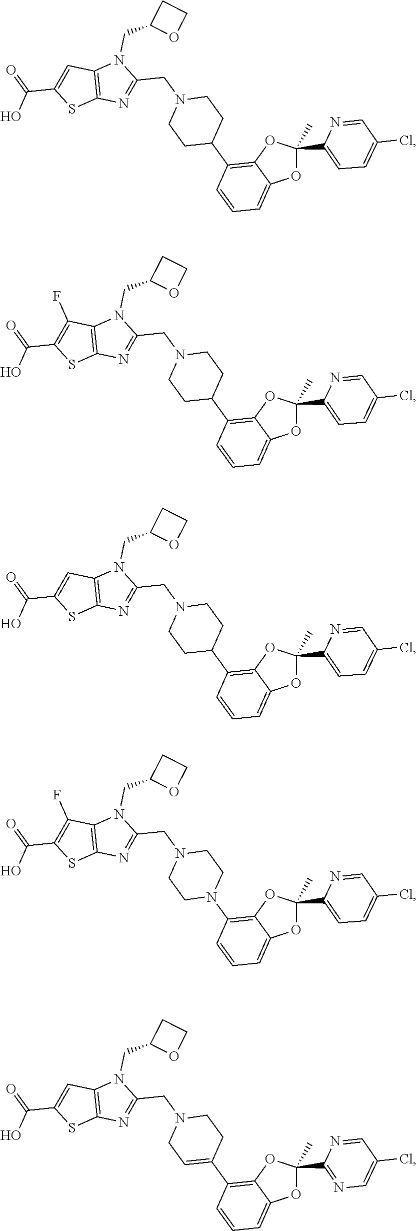 [SMILES: C[C@]1(c2ccc(Cl)cn2)Oc2cccc(C3CCN(Cc4nc5sc(C(=O)O)cc5n4C[C@@H]4CCO4)CC3)c2O1 ; C[C@]1(c2ccc(Cl)cn2)Oc2cccc(C3CCN(Cc4nc5sc(C(=O)O)c(F)c5n4C[C@@H]4CCO4)CC3)c2O1 ; C[C@]1(c2ccc(Cl)cn2)Oc2cccc(C3CCN(Cc4nc5sc(C(=O)O)cc5n4C[C@@H]4CCO4)CC3)c2O1 ; C[C@]1(c2ccc(Cl)cn2)Oc2cccc(N3CCN(Cc4nc5sc(C(=O)O)c(F)c5n4C[C@@H]4CCO4)CC3)c2O1 ; C[C@]1(c2ncc(Cl)cn2)Oc2cccc(C3=CCN(Cc4nc5sc(C(=O)O)cc5n4C[C@@H]4CCO4)CC3)c2O1]

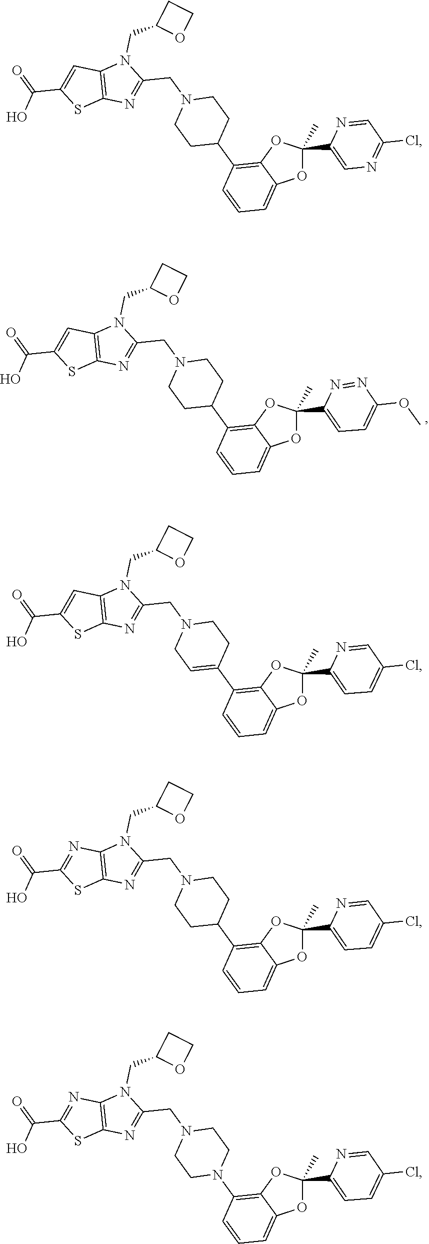 [SMILES: C[C@]1(c2cnc(Cl)cn2)Oc2cccc(C3CCN(Cc4nc5sc(C(=O)O)cc5n4C[C@@H]4CCO4)CC3)c2O1 ; COc1ccc([C@@]2(C)Oc3cccc(C4CCN(Cc5nc6sc(C(=O)O)cc6n5C[C@@H]5CCO5)CC4)c3O2)nn1 ; C[C@]1(c2ccc(Cl)cn2)Oc2cccc(C3=CCN(Cc4nc5sc(C(=O)O)cc5n4C[C@@H]4CCO4)CC3)c2O1 ; C[C@]1(c2ccc(Cl)cn2)Oc2cccc(C3CCN(Cc4nc5sc(C(=O)O)nc5n4C[C@@H]4CCO4)CC3)c2O1 ; C[C@]1(c2ccc(Cl)cn2)Oc2cccc(N3CCN(Cc4nc5sc(C(=O)O)nc5n4C[C@@H]4CCO4)CC3)c2O1]

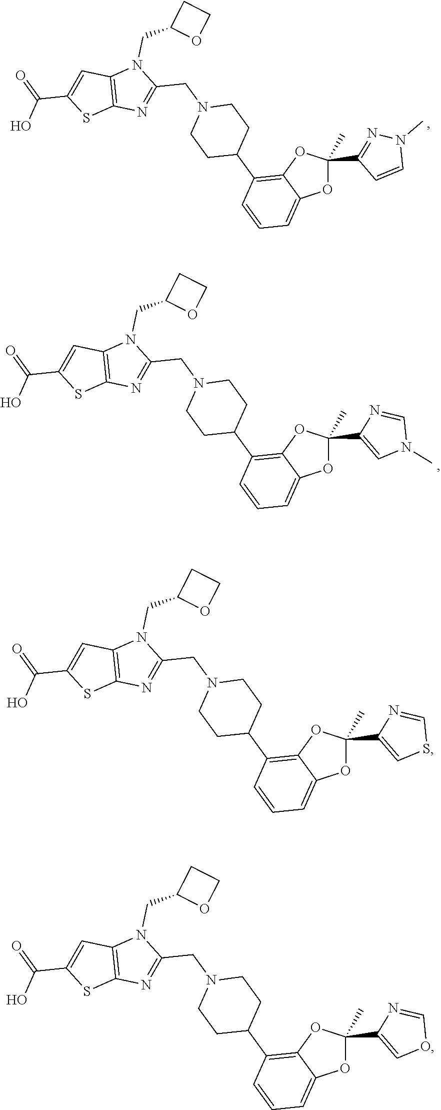 [SMILES: Cn1ccc([C@@]2(C)Oc3cccc(C4CCN(Cc5nc6sc(C(=O)O)cc6n5C[C@@H]5CCO5)CC4)c3O2)n1 ; Cn1cnc([C@@]2(C)Oc3cccc(C4CCN(Cc5nc6sc(C(=O)O)cc6n5C[C@@H]5CCO5)CC4)c3O2)c1 ; C[C@]1(c2cscn2)Oc2cccc(C3CCN(Cc4nc5sc(C(=O)O)cc5n4C[C@@H]4CCO4)CC3)c2O1 ; C[C@]1(c2cocn2)Oc2cccc(C3CCN(Cc4nc5sc(C(=O)O)cc5n4C[C@@H]4CCO4)CC3)c2O1]

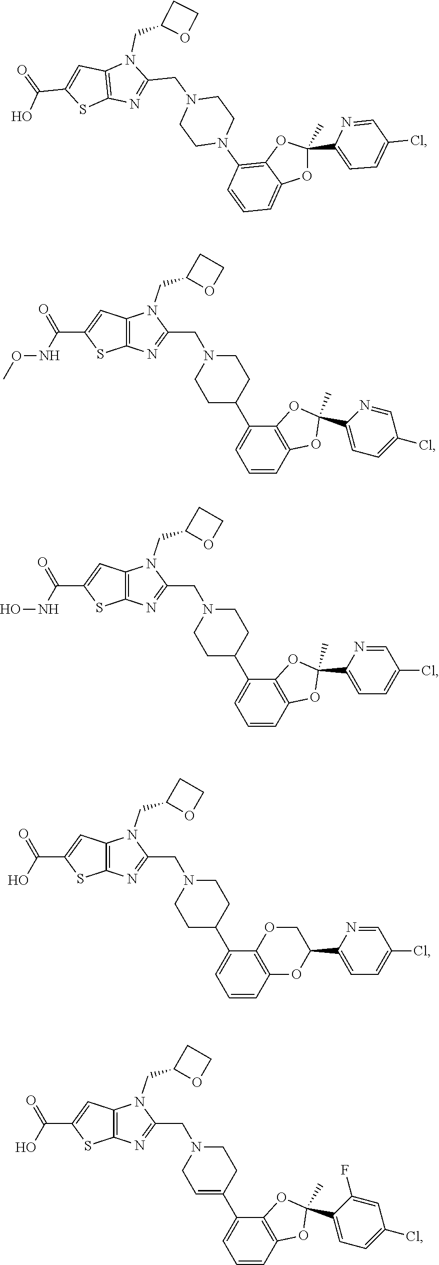 [SMILES: C[C@]1(c2ccc(Cl)cn2)Oc2cccc(N3CCN(Cc4nc5sc(C(=O)O)cc5n4C[C@@H]4CCO4)CC3)c2O1 ; CONC(=O)c1cc2c(nc(CN3CCC(c4cccc5c4O[C@@](C)(c4ccc(Cl)cn4)O5)CC3)n2C[C@@H]2CCO2)s1 ; C[C@]1(c2ccc(Cl)cn2)Oc2cccc(C3CCN(Cc4nc5sc(C(=O)NO)cc5n4C[C@@H]4CCO4)CC3)c2O1 ; O=C(O)c1cc2c(nc(CN3CCC(c4cccc5c4OC[C@@H](c4ccc(Cl)cn4)O5)CC3)n2C[C@@H]2CCO2)s1 ; C[C@]1(c2ccc(Cl)cc2F)Oc2cccc(C3=CCN(Cc4nc5sc(C(=O)O)cc5n4C[C@@H]4CCO4)CC3)c2O1]

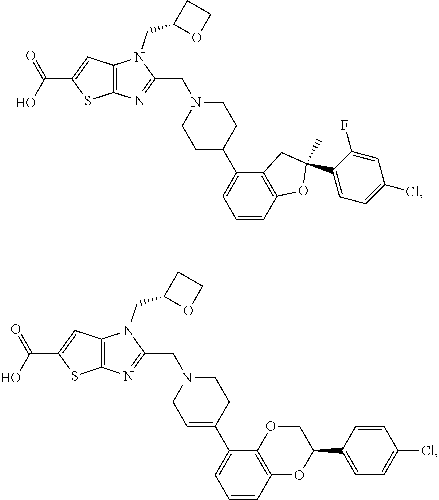 [SMILES: C[C@@]1(c2ccc(Cl)cc2F)Cc2c(cccc2C2CCN(Cc3nc4sc(C(=O)O)cc4n3C[C@@H]3CCO3)CC2)O1 ; O=C(O)c1cc2c(nc(CN3CC=C(c4cccc5c4OC[C@@H](c4ccc(Cl)cc4)O5)CC3)n2C[C@@H]2CCO2)s1]

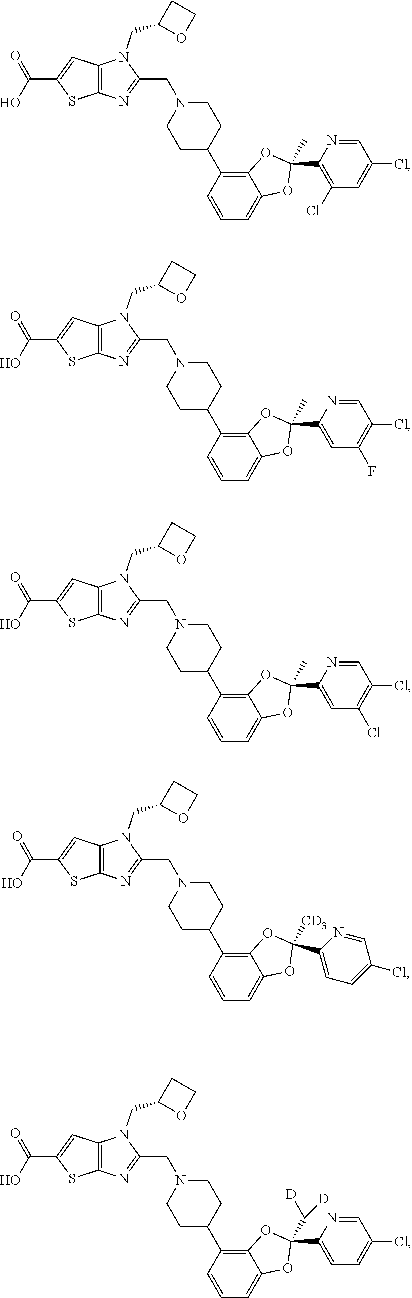 [SMILES: C[C@]1(c2ncc(Cl)cc2Cl)Oc2cccc(C3CCN(Cc4nc5sc(C(=O)O)cc5n4C[C@@H]4CCO4)CC3)c2O1 ; C[C@]1(c2cc(F)c(Cl)cn2)Oc2cccc(C3CCN(Cc4nc5sc(C(=O)O)cc5n4C[C@@H]4CCO4)CC3)c2O1 ; C[C@]1(c2cc(Cl)c(Cl)cn2)Oc2cccc(C3CCN(Cc4nc5sc(C(=O)O)cc5n4C[C@@H]4CCO4)CC3)c2O1 ; [2H]C([2H])([2H])[C@]1(c2ccc(Cl)cn2)Oc2cccc(C3CCN(Cc4nc5sc(C(=O)O)cc5n4C[C@@H]4CCO4)CC3)c2O1 ; [2H]C([2H])[C@]1(c2ccc(Cl)cn2)Oc2cccc(C3CCN(Cc4nc5sc(C(=O)O)cc5n4C[C@@H]4CCO4)CC3)c2O1]

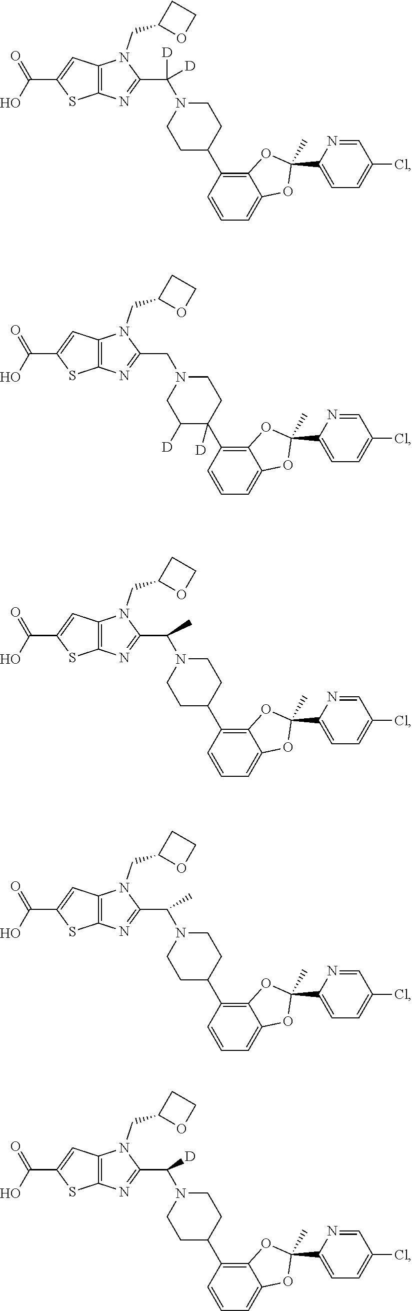 [SMILES: [2H]C([2H])(c1nc2sc(C(=O)O)cc2n1C[C@@H]1CCO1)N1CCC(c2cccc3c2O[C@@](C)(c2ccc(Cl)cn2)O3)CC1 ; [2H]C1CN(Cc2nc3sc(C(=O)O)cc3n2C[C@@H]2CCO2)CCC1([2H])c1cccc2c1O[C@@](C)(c1ccc(Cl)cn1)O2 ; C[C@H](c1nc2sc(C(=O)O)cc2n1C[C@@H]1CCO1)N1CCC(c2cccc3c2O[C@@](C)(c2ccc(Cl)cn2)O3)CC1 ; C[C@@H](c1nc2sc(C(=O)O)cc2n1C[C@@H]1CCO1)N1CCC(c2cccc3c2O[C@@](C)(c2ccc(Cl)cn2)O3)CC1 ; [2H][C@H](c1nc2sc(C(=O)O)cc2n1C[C@@H]1CCO1)N1CCC(c2cccc3c2O[C@@](C)(c2ccc(Cl)cn2)O3)CC1]

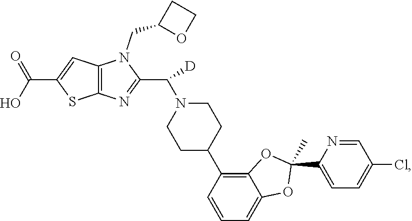 [SMILES: [2H][C@@H](c1nc2sc(C(=O)O)cc2n1C[C@@H]1CCO1)N1CCC(c2cccc3c2O[C@@](C)(c2ccc(Cl)cn2)O3)CC1]

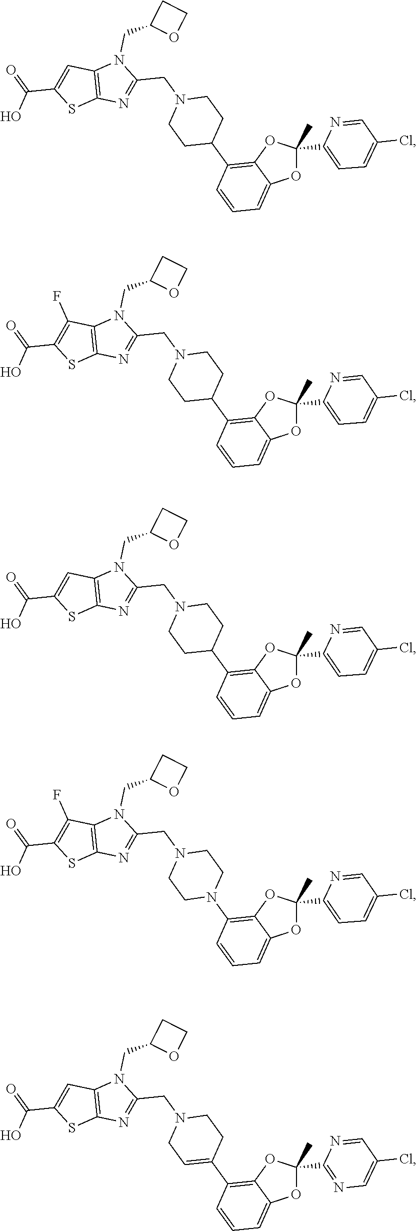 [SMILES: C[C@@]1(c2ccc(Cl)cn2)Oc2cccc(C3CCN(Cc4nc5sc(C(=O)O)cc5n4C[C@@H]4CCO4)CC3)c2O1 ; C[C@@]1(c2ccc(Cl)cn2)Oc2cccc(C3CCN(Cc4nc5sc(C(=O)O)c(F)c5n4C[C@@H]4CCO4)CC3)c2O1 ; C[C@@]1(c2ccc(Cl)cn2)Oc2cccc(C3CCN(Cc4nc5sc(C(=O)O)cc5n4C[C@@H]4CCO4)CC3)c2O1 ; C[C@@]1(c2ccc(Cl)cn2)Oc2cccc(N3CCN(Cc4nc5sc(C(=O)O)c(F)c5n4C[C@@H]4CCO4)CC3)c2O1 ; C[C@@]1(c2ncc(Cl)cn2)Oc2cccc(C3=CCN(Cc4nc5sc(C(=O)O)cc5n4C[C@@H]4CCO4)CC3)c2O1]

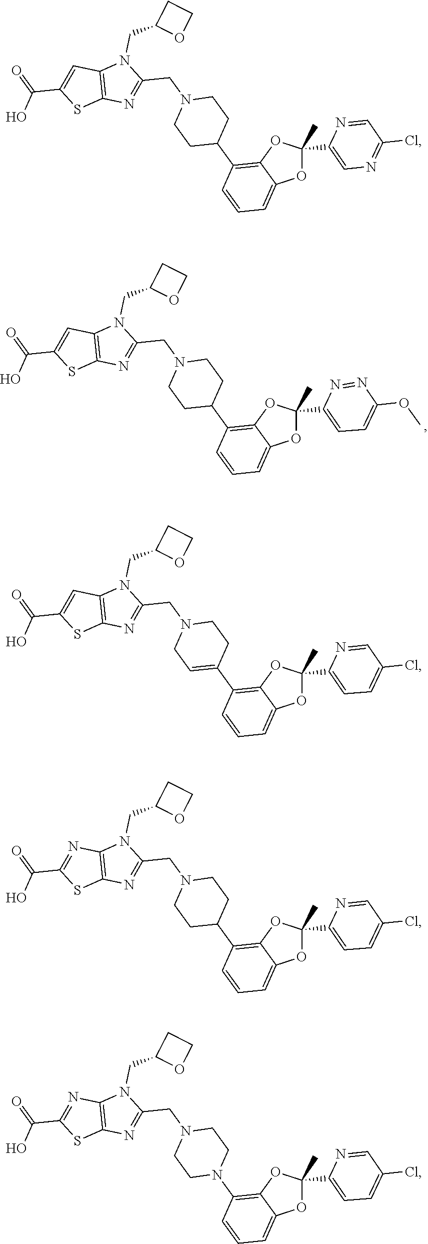 [SMILES: C[C@@]1(c2cnc(Cl)cn2)Oc2cccc(C3CCN(Cc4nc5sc(C(=O)O)cc5n4C[C@@H]4CCO4)CC3)c2O1 ; COc1ccc([C@]2(C)Oc3cccc(C4CCN(Cc5nc6sc(C(=O)O)cc6n5C[C@@H]5CCO5)CC4)c3O2)nn1 ; C[C@@]1(c2ccc(Cl)cn2)Oc2cccc(C3=CCN(Cc4nc5sc(C(=O)O)cc5n4C[C@@H]4CCO4)CC3)c2O1 ; C[C@@]1(c2ccc(Cl)cn2)Oc2cccc(C3CCN(Cc4nc5sc(C(=O)O)nc5n4C[C@@H]4CCO4)CC3)c2O1 ; C[C@@]1(c2ccc(Cl)cn2)Oc2cccc(N3CCN(Cc4nc5sc(C(=O)O)nc5n4C[C@@H]4CCO4)CC3)c2O1]

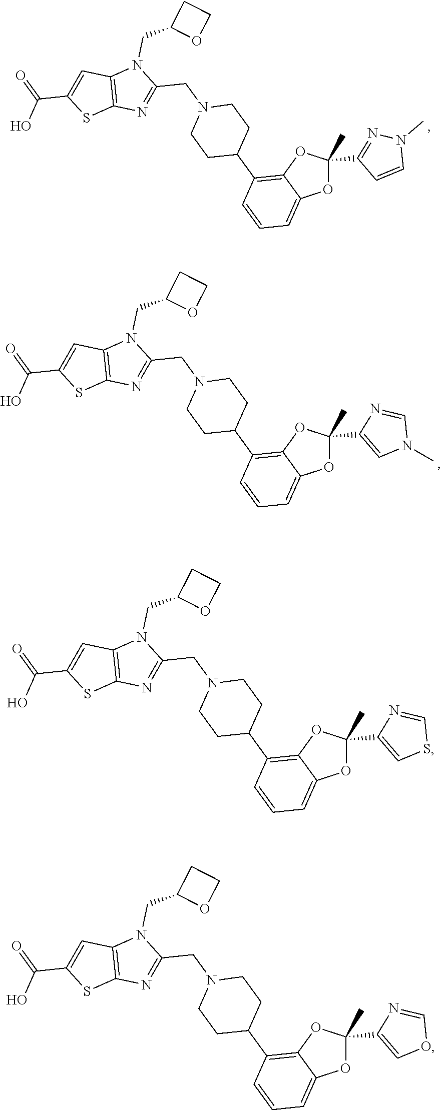 [SMILES: Cn1ccc([C@]2(C)Oc3cccc(C4CCN(Cc5nc6sc(C(=O)O)cc6n5C[C@@H]5CCO5)CC4)c3O2)n1 ; Cn1cnc([C@]2(C)Oc3cccc(C4CCN(Cc5nc6sc(C(=O)O)cc6n5C[C@@H]5CCO5)CC4)c3O2)c1 ; C[C@@]1(c2cscn2)Oc2cccc(C3CCN(Cc4nc5sc(C(=O)O)cc5n4C[C@@H]4CCO4)CC3)c2O1 ; C[C@@]1(c2cocn2)Oc2cccc(C3CCN(Cc4nc5sc(C(=O)O)cc5n4C[C@@H]4CCO4)CC3)c2O1]

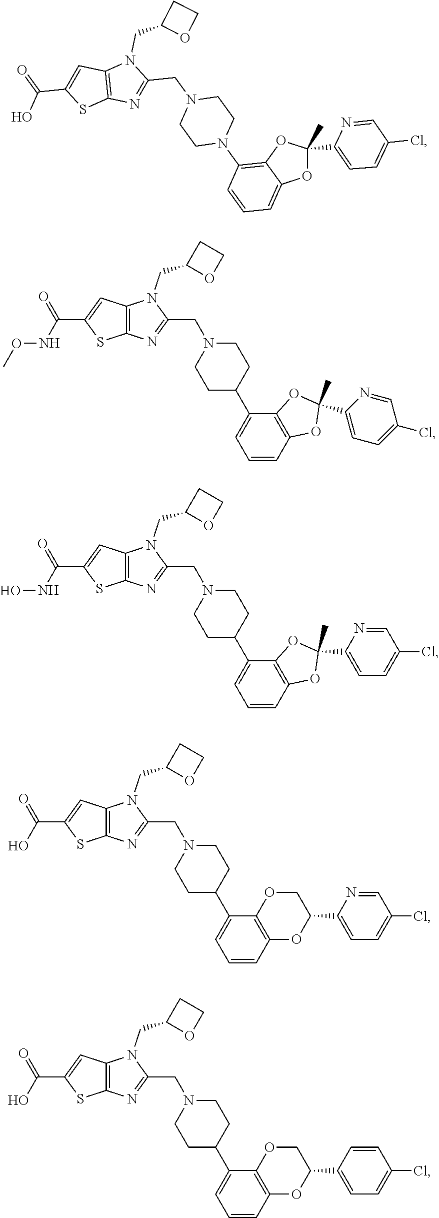 [SMILES: C[C@@]1(c2ccc(Cl)cn2)Oc2cccc(N3CCN(Cc4nc5sc(C(=O)O)cc5n4C[C@@H]4CCO4)CC3)c2O1 ; CONC(=O)c1cc2c(nc(CN3CCC(c4cccc5c4O[C@](C)(c4ccc(Cl)cn4)O5)CC3)n2C[C@@H]2CCO2)s1 ; C[C@@]1(c2ccc(Cl)cn2)Oc2cccc(C3CCN(Cc4nc5sc(C(=O)NO)cc5n4C[C@@H]4CCO4)CC3)c2O1 ; O=C(O)c1cc2c(nc(CN3CCC(c4cccc5c4OC[C@H](c4ccc(Cl)cn4)O5)CC3)n2C[C@@H]2CCO2)s1 ; O=C(O)c1cc2c(nc(CN3CCC(c4cccc5c4OC[C@H](c4ccc(Cl)cc4)O5)CC3)n2C[C@@H]2CCO2)s1]

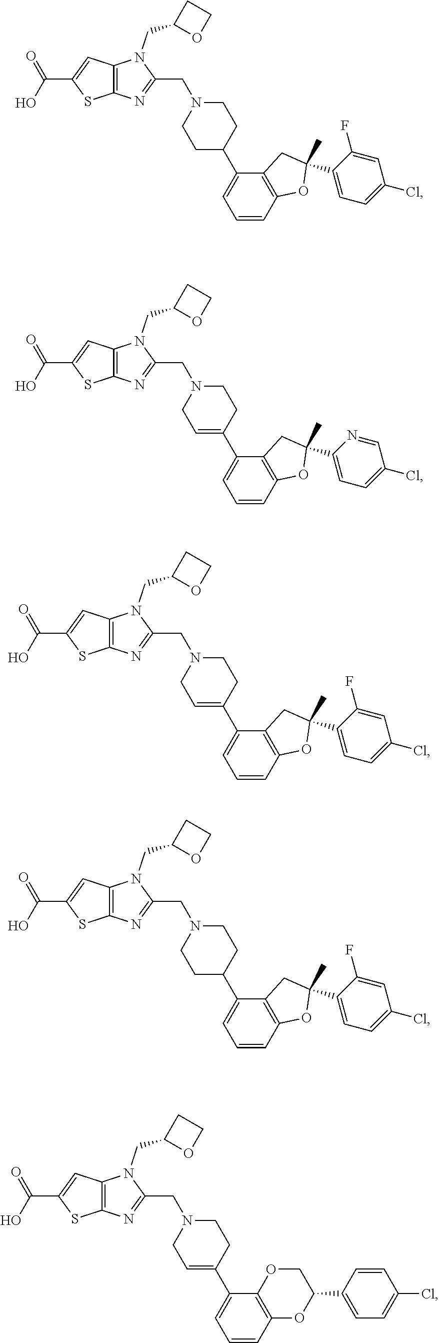 [SMILES: C[C@]1(c2ccc(Cl)cc2F)Cc2c(cccc2C2CCN(Cc3nc4sc(C(=O)O)cc4n3C[C@@H]3CCO3)CC2)O1 ; C[C@]1(c2ccc(Cl)cn2)Cc2c(cccc2C2=CCN(Cc3nc4sc(C(=O)O)cc4n3C[C@@H]3CCO3)CC2)O1 ; C[C@]1(c2ccc(Cl)cc2F)Cc2c(cccc2C2=CCN(Cc3nc4sc(C(=O)O)cc4n3C[C@@H]3CCO3)CC2)O1 ; C[C@]1(c2ccc(Cl)cc2F)Cc2c(cccc2C2CCN(Cc3nc4sc(C(=O)O)cc4n3C[C@@H]3CCO3)CC2)O1 ; O=C(O)c1cc2c(nc(CN3CC=C(c4cccc5c4OC[C@H](c4ccc(Cl)cc4)O5)CC3)n2C[C@@H]2CCO2)s1]

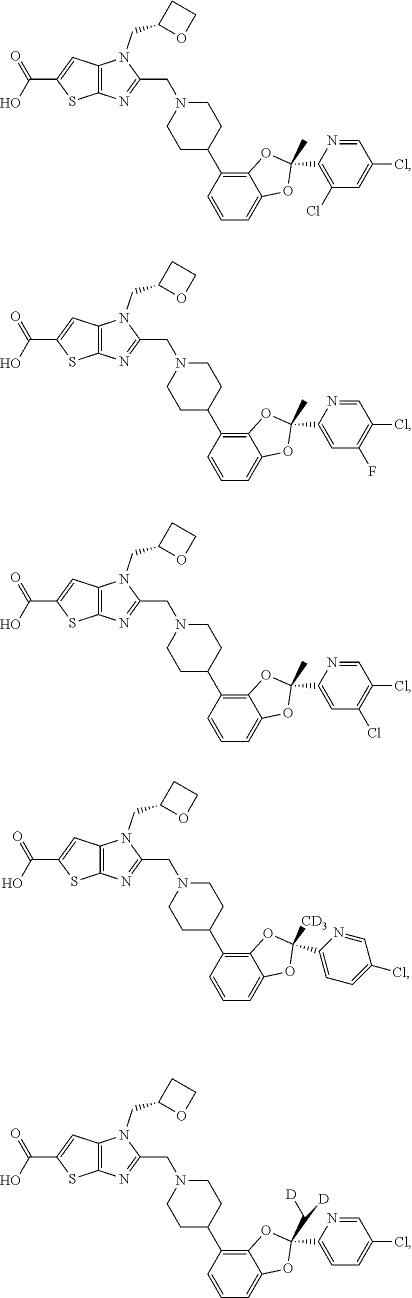 [SMILES: C[C@@]1(c2ncc(Cl)cc2Cl)Oc2cccc(C3CCN(Cc4nc5sc(C(=O)O)cc5n4C[C@@H]4CCO4)CC3)c2O1 ; C[C@@]1(c2cc(F)c(Cl)cn2)Oc2cccc(C3CCN(Cc4nc5sc(C(=O)O)cc5n4C[C@@H]4CCO4)CC3)c2O1 ; C[C@@]1(c2cc(Cl)c(Cl)cn2)Oc2cccc(C3CCN(Cc4nc5sc(C(=O)O)cc5n4C[C@@H]4CCO4)CC3)c2O1 ; [2H]C([2H])([2H])[C@@]1(c2ccc(Cl)cn2)Oc2cccc(C3CCN(Cc4nc5sc(C(=O)O)cc5n4C[C@@H]4CCO4)CC3)c2O1 ; [2H]C([2H])[C@@]1(c2ccc(Cl)cn2)Oc2cccc(C3CCN(Cc4nc5sc(C(=O)O)cc5n4C[C@@H]4CCO4)CC3)c2O1]

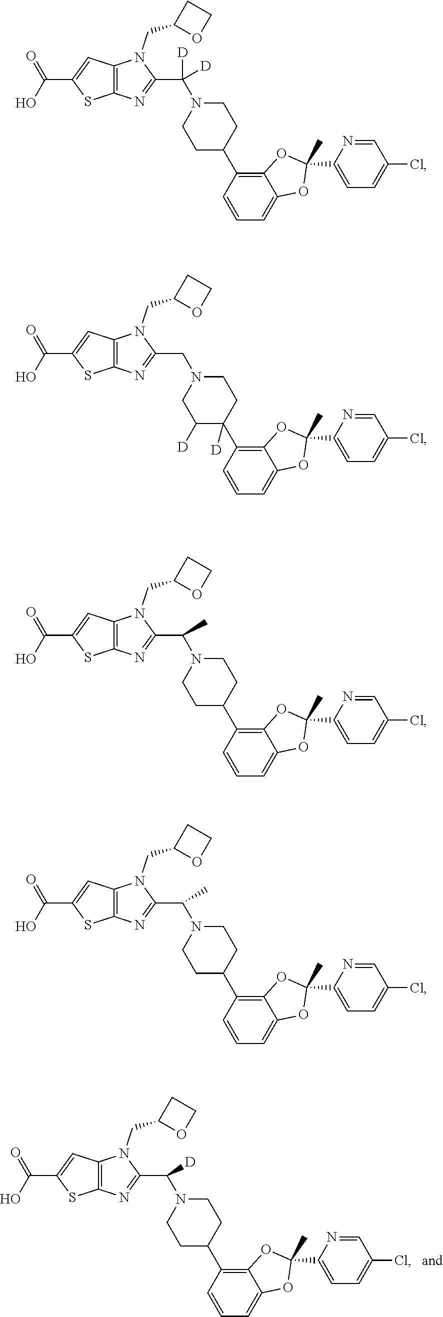 [SMILES: [2H]C([2H])(c1nc2sc(C(=O)O)cc2n1C[C@@H]1CCO1)N1CCC(c2cccc3c2O[C@](C)(c2ccc(Cl)cn2)O3)CC1 ; [2H]C1CN(Cc2nc3sc(C(=O)O)cc3n2C[C@@H]2CCO2)CCC1([2H])c1cccc2c1O[C@](C)(c1ccc(Cl)cn1)O2 ; C[C@H](c1nc2sc(C(=O)O)cc2n1C[C@@H]1CCO1)N1CCC(c2cccc3c2O[C@](C)(c2ccc(Cl)cn2)O3)CC1 ; C[C@@H](c1nc2sc(C(=O)O)cc2n1C[C@@H]1CCO1)N1CCC(c2cccc3c2O[C@](C)(c2ccc(Cl)cn2)O3)CC1 ; [2H][C@H](c1nc2sc(C(=O)O)cc2n1C[C@@H]1CCO1)N1CCC(c2cccc3c2O[C@](C)(c2ccc(Cl)cn2)O3)CC1 ; "and"]

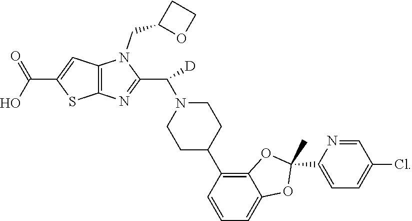 [SMILES: [2H][C@@H](c1nc2sc(C(=O)O)cc2n1C[C@@H]1CCO1)N1CCC(c2cccc3c2O[C@](C)(c2ccc(Cl)cn2)O3)CC1]

18. A method for treating diabetes in a subject in need thereof, comprising: administering the compound or the pharmaceutically acceptable salt thereof according to claim 1 to the subject.

19. The compound or the pharmaceutically acceptable salt thereof according to claim 1, wherein R₂ is selected from 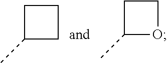
or, ring B is selected from

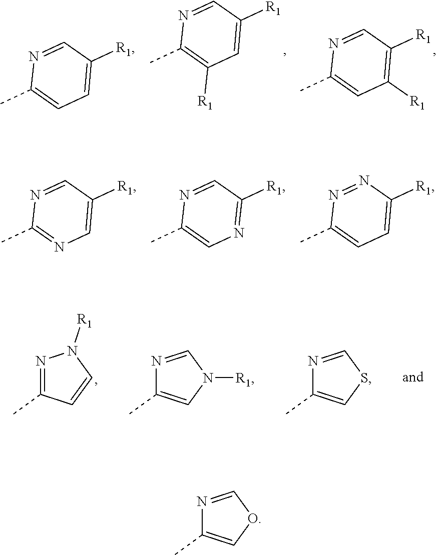

20. The compound or the pharmaceutically acceptable salt thereof according to claim 11, wherein ring B is selected from 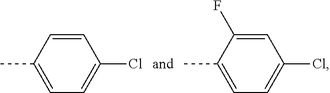
and the structural moiety 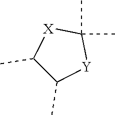
is selected from [chemical structure]
